(19) United States
(12) Patent Application Publication
(10) Pub. No.: US 2003/0190640 A1
(43) Pub. Date: Oct. 9, 2003

(54) Genes expressed in prostate cancer

(76) Inventors: Mary Faris, Los Angeles, CA (US); Cecelia I. Pearson, Palo Alto, CA (US)

Correspondence Address: INCYTE GENOMICS, INC., 3160 Porter Drive, Palo Alto, CA, 94304, US

(21) Appl. No.: 10/252,157

(22) Filed: May 29, 2002

Related U.S. Application Data
(60) Provisional application No. 60/295,048, filed on May 31, 2001.

(51) Int. Cl. C12Q 1/68; C07H 21/04

(52) U.S. Cl. 435/6; 536/24.3

(57) ABSTRACT

The present invention relates to a combination comprising a plurality of cDNAs which are differentially expressed in prostate cancer and which may be used in their entirety or in part as to diagnose, to stage to treat or to monitor the progression or treatment of prostate cancer.

This application claims the benefit of Provisional Application No. 60,295,048 filed May 31, 2001.

### FIELD OF THE INVENTION

The present invention relates to a combination comprising a plurality of cDNAs which are differentially expressed in prostate cancer and which may be used entirely or in part to diagnose, to stage, to treat, or to monitor the progression or treatment of prostate cancer.

### BACKGROUND OF THE INVENTION

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for examining which genes are tissue specific, carrying out housekeeping functions, parts of a signaling cascade, or specifically related to a particular genetic predisposition, condition, disease, or disorder.

The potential application of gene expression profiling is particularly relevant to improving diagnosis, prognosis, and treatment of disease. For example, both the levels and sequences expressed in tissues from subjects with prostate cancer may be compared with the levels and sequences expressed in normal tissue.

Prostate cancer is a common malignancy in men over the age of 50, and the incidence increases with age. In the US, there are approximately 132,000 newly diagnosed cases of prostate cancer and more than 33,000 deaths from the disorder each year.

Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most cancers, prostate cancer develops through a multistage progression ultimately resulting in an aggressive, metastatic phenotype. The initial step in tumor progression involves the hyperproliferation of normal luminal and/or basal epithelial cells that become hyperplastic and evolve into early-stage tumors. The early-stage tumors are localized in the prostate but eventually may metastasize, particularly to the bone, brain or lung. About 80% of these tumors remain responsive to androgen treatment, an important hormone controlling the growth of prostate epithelial cells. However, in its most advanced state, cancer growth becomes androgen-independent and there is currently no known treatment for this condition.

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Current areas of cancer research provide additional prospects for markers as well as potential therapeutic targets for prostate cancer. Several growth factors have been shown to play a critical role in tumor development, growth, and progression. The growth factors Epidermal Growth Factor (EGF), Fibroblast Growth Factor (FGF), and Tumor Growth Factor alpha (TGFα) are important in the growth of normal as well as hyperproliferative prostate epithelial cells, particularly at early stages of tumor development and progression, and affect signaling pathways in these cells in various ways (Lin et al. (1999) Cancer Res 59:2891-2897; Putz et al. (1999) Cancer Res 59:227-233). The TGF-β family of growth factors are generally expressed at increased levels in human cancers and the high expression levels in many cases correlates with advanced stages of malignancy and poor survival (Gold (1999) Crit Rev Oncog 10:303-360). Finally, there are human cell lines representing both the androgen-dependent stage of prostate cancer (LNCap) as well as the androgen-independent, hormone refractory stage of the disease (PC3 and DU-145) that have proved useful in studying gene expression patterns associated with the progression of prostate cancer, and the effects of cell treatments on these expressed genes (Chung (1999) Prostate 15:199-207).

The present invention provides a combination comprising a plurality of cDNAs and individual cDNAs that can be employed for the diagnosis, prognosis or treatment of prostate cancer. The present invention satisfies a need in the art in that it provides a set of differentially expressed cDNAs which may be used entirely or in part to diagnose, to stage, to treat, or to monitor the progression or treatment of a subject with prostate cancer.

### SUMMARY

The present invention provides a combination comprising a plurality of cDNAs and their complements which are expressed in prostate cancer and which are selected from SEQ ID NOs:1-501 as presented in the Sequence Listing. In one embodiment, each cDNA, specifically SEQ ID NOs: 1-56, 87-153, 164-349, 370-414, and 437-501, is downregulated at least two-fold; in another embodiment, each cDNA, specifically SEQ ID NOs:57-86, 154-163, 350-369, and 415-436, is upregulated at least two-fold. In one aspect, the combination is useful to diagnose or treat prostate cancer. In another aspect, the combination is immobilized on a substrate.

The invention also provides an isolated cDNA selected from SEQ ID NOs:14, 26, 40, 52, 55, 60, 65, 68, 73, 79, 82, 85, 92, 110, 112, 114, 115, 117, 122, 125, 126, 130, 136, 137, 139, 141, 143, 144, 145, 146, 147, 160, 164, 166, 167, 168, 190, 191, 194, 195, 199, 201, 204, 211, 212, 222, 224, 226, 229, 233, 234, 240, 243, 245, 248, 250, 253, 254, 259, 264, 268, 269, 270, 272, 276, 278, 279, 281, 282, 284, 285, 286, 293, 296, 297, 299, 300, 301, 302, 306, 308, 312, 313, 314, 317, 319, 321, 321, 322, 322, 323, 324, 325, 326, 330, 331, 332, 334, 336, 337, 338, 339, 340, 342, 346, 353, 355, 357, 361, 365, 366, 371, 372, 376, 380, 383, 385, 386, 387, 390, 399, 400, 402, 405, 406, 408, 409, 410, 412, 413, 417, 418, 419, 420, 422, 422, 424, 426, 438, 444, 445, 453, 456, 460, 461, 471, 479, 480, 487, 490, 492, 495, 496, and 497 as presented in the Sequence Listing. The invention also provides a vector comprising the cDNA, a host cell comprising the vector, and a method for producing a protein comprising culturing the host cell under conditions for the expression of a protein and recovering the protein from the host cell culture.

The invention further provides a method to detect differential expression of one or more of the cDNAs of the combination, the method comprising: hybridizing the substrate comprising the combination with the nucleic acids of a sample, thereby forming one or more hybridization complexes, detecting the hybridization complexes, and comparing the hybridization complexes with those of a standard, wherein differences in the size and signal intensity of each hybridization complex indicates differential expression of nucleic acids in the sample. In one aspect, the sample is from prostate, and differential expression determines an early, mid, and late stage of the disorder.

The invention still further provides a method of screening a library or a plurality of molecules or compounds to identify a ligand, the method comprising: combining the substrate comprising the combination with a library or a plurality of molecules or compounds under conditions to allow specific binding and detecting specific binding, thereby identifying a ligand. The library or a plurality of molecules or compounds are selected from DNA molecules, RNA molecules, peptide nucleic acid molecules, mimetics, peptides, transcription factors, repressors, and other regulatory proteins. The invention additionally provides a method for purifying a ligand, the method comprising combining a cDNA of the invention with a sample under conditions which allow specific binding, recovering the bound cDNA, and separating the cDNA from the ligand, thereby obtaining purified ligand.

The present invention provides a purified protein encoded and produced by a cDNA of the invention. The invention also provides a method for using a protein to screen a library or a plurality of molecules or compounds to identify a ligand, the method comprising: combining the protein or a portion thereof with the library or a plurality of molecules or compounds under conditions to allow specific binding and detecting specific binding, thereby identifying a ligand which specifically binds the protein. A library or a plurality of molecules or compounds are selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents. The invention further provides for using a protein to purify a ligand, the method comprising: combining the protein or a portion thereof with a sample under conditions to allow specific binding, recovering the bound protein, and separating the protein from the ligand, thereby obtaining purified ligand. The invention still further provides a composition comprising the protein and a pharmaceutical carrier. The invention yet still further provides a method for using the protein to produce an antibody, the method comprising: immunizing an animal with the protein or an antigenic determinant under conditions to elicit an antibody response, isolating animal antibodies, and screening the isolated antibodies with the protein to identify an antibody which specifically binds the protein. The invention even further provides a method for using the protein to purify antibodies which bind specifically to the protein.

The present invention provides a purified antibody. The invention also provides a method of using an antibody to detect the expression of a protein in a sample, the method comprising contacting the antibody with a sample under conditions for the formation of an antibody:protein complex and detecting complex formation wherein the formation of the complex indicates the expression of the protein in the sample. In one aspect, the sample is from prostate. In another aspect, complex formation is compared to standards and is diagnostic of prostate cancer. The invention further provides using an antibody to immunopurify a protein comprising combining the antibody with a sample under conditions to allow formation of an antibody:protein complex, and separating the antibody from the protein, thereby obtaining purified protein. The invention still further provides a method of using an antibody to detect prostate cancer, the method comprises contacting a sample with the antibody which specifically binds a protein of the invention under conditions to form an antibody:protein complex, detecting antibody:protein complex formation, and comparing complex formation with standards, wherein complex formation indicates the presence of prostate cancer in the sample.

The invention provides a composition comprising a cDNA, a protein, an antibody, or a ligand with agonistic or antagonistic activity that can be used in the methods of the invention or to treat prostate cancer.

### DESCRIPTION OF THE COMPACT DISC-RECORDABLE (CD-R)

CD-R 1 is labeled: “PA-0027-1 US, Copy 1,” was created on May 29, 2002 and contains: the Sequence Listing formatted in plain ASCII text. The file for the Sequence Listing is entitled pa00271us_seqlist.txt, created on May 29, 2002 and is 1,430 KB in size.

CD-R 2 is an exact copy of CD-R 1. CD-R 2 is labeled: “PA-0027-1 US, Copy 2,” and was created on May 29, 2002.

The CD-R labeled as: “PA-0027-1 US, CRF,” contains the Sequence Listing formatted in plain ASCII text. The file for the Sequence Listing is entitled pa00291us_seqlist.txt, was created on May 29, 2002 and is 1,430 KB in size.

The content of the Sequence Listing named above and as described below, submitted in duplicate on two (2) CD-Rs (labeled “PA-0027-1 US, Copy 1” and “PA-0027-1 US, Copy 2”), and the CRF (labeled “PA-0027-1 US, CRF”) containing the Sequence Listing, are incorporated by reference herein, in their entirety.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of cDNAs obtained by sequencing and extension of clone inserts. Each sequence is identified by a sequence identification number (SEQ ID NO) and by the template number (TEMPLATE ID) from which it was obtained.

Table 1 lists the functional annotation and differential expression of the cDNAs of the present invention. Columns 1 and 2 show the SEQ ID NO and TEMPLATE ID, respectively. Columns 3, 4, and 5 show the GenBank hit (GI Number), probability score (E-value), and functional annotation, respectively, as determined by BLAST analysis (version 1.4 using default parameters; Altschul (1993) J Mol Evol 36:290-300; Altschul et al. (1990) J Mol Biol 215:403-410) of the cDNA against GenBank (release 116; National Center for Biotechnology Information (NCBI), Bethesda Md.). Column 6 shows the prostate cancer cell line in which differential expression was observed. The table is further subdivided by the treatment group and up- or down-regulated expression.

Table 2 shows Pfam annotations for proteins encoded by the cDNAs of the present invention. Columns 1 and 2 show the SEQ ID NO and TEMPLATE ID of each cDNA, respectively. Columns 3, 4, and 5 show the first nucleotide (START), last nucleotide (STOP), and reading frame, respectively, for the protein encoded by the cDNA as identified by Pfam analysis of the encoded protein. Columns 6 and 7 show the Pfam description and E-values, respectively, corresponding to the protein domain encoded by the cDNA.

Table 3 shows signal peptide and transmembrane motifs predicted for the protein encoded by the cDNAs of the present invention. Columns 1 and 2 show the SEQ ID NO and TEMPLATE ID of each cDNA, respectively. Columns 3, 4, and 5 show the first nucleotide (START), last nucleotide (STOP), and reading frame, respectively, for the protein encoded by the cDNA, and column 6 identifies the signal peptide (SP) or transmembrane (TM) domain for the encoded protein.

Table 4 shows the region of each cDNA encompassed by the clone present on a microarray and identified as differentially expressed. Columns 1 and 2 show the SEQ ID NO and TEMPLATE ID of each cDNA, respectively. Column 3 shows the CLONE ID and columns 4 and 5 show the first nucleotide (START) and last nucleotide (STOP) encompassed by the clone on the template.

### DESCRIPTION OF THE INVENTION

Definitions “Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein. “Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity. “Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

A “combination” refers to at least two and up to about 1002 cDNAs wherein the cDNAs are SEQ ID NOs: 1-501 as presented in the Sequence Listing and the complements thereof.

The “complement” of a cDNA of the Sequence Listing refers to a nucleic acid molecule which is completely complementary over its full length and which will hybridize to a nucleic acid molecule under conditions of high stringency. “cDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Altschul, supra; Altschul et al., supra) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like. “Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer longer lifespan or enhanced activity. “Differential expression” refers to an increased or upregulated or a decreased or downregulated expression as detected by absence, presence, or at least two-fold change in the amount of transcribed messenger RNA or translated protein in a sample. “Disorder” refers to neoplastic conditions and diseases such as cancer, and in particular, prostate cancer.

An “expression profile” is a representation of gene expression in a sample. A nucleic acid expression profile is produced using sequencing, hybridization, or amplification technologies using mRNAs or cDNAs from a sample. A protein expression profile, although time delayed, mirrors the nucleic acid expression profile and is produced using gel electrophoresis, mass spectrometry, or an array and labeling moieties or antibodies which specifically bind the protein. The nucleic acids, proteins, or antibodies specifically binding the protein may be used in solution or attached to a substrate. “Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions. “Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997, supra). BLAST2 may be used in a standardized and reproducible way to insert gaps in one of the sequences in order to optimize alignment and to achieve a more meaningful comparison between them. “Similarity” uses the same algorithms but takes conservative substitution of residues into account. In proteins, similarity exceeds identity in that substitution of a valine for a leucine or isoleucine, is counted in calculating the reported percentage. Substitutions which are considered to be conservative are well known in the art. “Isolated or “purified” refers to any molecule or compound that is separated from its natural environment and is from about 60% free to about 90% free from other components with which it is naturally associated. “Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels include but are not limited to anthocyanins, fluorescein, green fluorescent protein (GFP), β glucuronidase, lissamine, luciferase, phycoerythrin, rhodamine, Cy3 and Cy5, and the like. Radioactive markers include radioactive forms of hydrogen, iodine, phosphorous, sulfur, and the like. “Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids. “Oligonucleotide” refers a single-stranded molecule from about 18 to about 60 nucleotides in length which may be used in hybridization or amplification technologies or in regulation of replication, transcription or translation. Equivalent terms are amplicon, amplimer, primer, and oligomer. “Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like. “Probe” refers to a cDNA that hybridizes to a nucleic acid or specifically binds to a ligand. Probes can be labeled with reporter molecules for use in hybridization technologies including Southern, northern, in situ, dot blot, and array, or in screening assays. “Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody. “Sample” is used in its broadest sense as containing nucleic acids, proteins, and antibodies. A sample may comprise a bodily fluid such as ascites, blood, cerebrospinal fluid, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like. “Specific binding” refers to a precise interaction between two molecules which is dependent upon their structure, particularly their molecular side groups. For example, the intercalation of a regulatory protein into the major groove of a DNA molecule or the binding between an epitope of a protein and an agonist, antagonist, or antibody. “Substrate” refers to any rigid or semi-rigid support to which cDNAs, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference. “Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure. The Invention The present invention provides for a combination comprising a plurality of cDNAs or their complements, SEQ ID NOs: 1-501, which are differentially expressed in human prostate cancer cells treated with various growth factors and growth regulators relative to the untreated cells and to normal human prostate epithelial cells similarily treated, and which may be used to diagnose, to stage, to treat or to monitor the progression or treatment of prostate cancer. The composition may be used in its entirety or in part, as subsets of downregulated cDNAs, SEQ ID NOs: 1-56, 87-153, 164-349, 370-414, and 437-501, or of upregulated cDNAs, SEQ ID NOs:57-86, 154-163, 350-369, and 415-436.

SEQ ID NOs: 14, 26, 40, 52, 55, 60, 65, 68, 73, 79, 82, 85, 92, 110, 112, 114, 115, 117, 122, 125, 126, 130, 136, 137, 139, 141, 143, 144, 145, 146, 147, 160, 164, 166, 167, 168, 190, 191, 194, 195, 199, 201, 204, 211, 212, 222, 224, 226, 229, 233, 234, 240, 243, 245, 248, 250, 253, 254, 259, 264, 268, 269, 270, 272, 276, 278, 279, 281, 282, 284, 285, 286, 293, 296, 297, 299, 300, 301, 302, 306, 308, 312, 313, 314, 317, 319, 321, 321, 322, 322, 323, 324, 325, 326, 330, 331, 332, 334, 336, 337, 338, 339, 340, 342, 346, 353, 355, 357, 361, 365, 366, 371, 372, 376, 380, 383, 385, 386, 387, 390, 399, 400, 402, 405, 406, 408, 409, 410, 412, 413, 417, 418, 419, 420, 422, 422, 424, 426, 438, 444, 445, 453, 456, 460, 461, 471, 479, 480, 487, 490, 492, 495, 496, and 497 represent novel cDNAs differentially expressed in prostate cancer cells. Since the novel cDNAs were identified solely by their differential expression, it is not essential to know a priori the name, structure, or function of the gene or it's encoded protein. The usefulness of the novel cDNAs exists in their immediate value as diagnostics for prostate cancer.

Table 1 shows those cDNAs having lower expression (two-fold or greater decrease) or higher expression (two-fold or greater increase) in prostate cancer cells following various growth factor and growth regulator treatments. Table 2 shows Pfam annotations of the cDNAs of the present invention. Columns 1 and 2 show the SEQ ID NO and TEMPLATE ID, respectively. Columns 3, 4, and 5 show the first nucleotide (START), last nucleotide (STOP), and reading frame, respectively, for the protein encoded by the cDNA and identified by Pfam analysis. Columns 6 and 7 show the Pfam description and E-values, respectively, corresponding to the protein domain encoded by the cDNA. Table 3 shows signal peptide and transmembrane regions predicted within the proteins encoded by the cDNAs of the present invention. Columns 1 and 2 show the SEQ ID NO and TEMPLATE ID, respectively. Columns 3, 4, and 5 show the first nucleotide (START), last nucleotide (STOP), and reading frame, respectively, for a protein encoded by the cDNA, and column 6 identifies the signal peptide (SP) or transmembrane (TM) domain of the protein. Table 4 shows the region of each cDNA encompassed by the clone present on a microarray and identified as differentially expressed. Columns 1 and 2 show the SEQ ID NO and TEMPLATE ID, respectively. Column 3 shows the CLONE ID and columns 4 and 5 show the first nucleotide (START) and last nucleotide (STOP) encompassed by the clone on the template.

The differential expression of the cDNAs as shown using microarray analysis define an expression profile for prostate cancer. Experimentally, differential expression of the cDNAs can be evaluated by methods including, but not limited to, differential display by spatial immobilization or by gel electrophoresis, genome mismatch scanning, representational discriminant analysis, clustering, transcript imaging and array technologies. These methods may be used alone or in combination.

The combination may be arranged on a substrate and hybridized with tissues from prostate cancer patients to identify which of the cDNAs are differentially expressed. If the patient has a known stage of prostate cancer, i.e., metastasis to brain, bone, or lung, this allows identification of those sequences of highest potential therapeutic value. In one embodiment, an additional set of cDNAs, such as cDNAs encoding signaling molecules, are arranged on the substrate with the combination. Such combinations may be useful in the elucidation of pathways which are affected in prostate cancer or to identify new, coexpressed, candidate, therapeutic molecules.

In another embodiment, the combination can be used for large scale genetic or gene expression analysis of a large number of novel, nucleic acid molecules. These samples are prepared by methods well known in the art and are from mammalian cells or tissues which are in a certain stage of development; have been treated with a known molecule or compound, such as a cytokine, growth factor, a drug, and the like; or have been extracted or biopsied from a mammal with a known or unknown condition, disorder, or disease before or after treatment. The sample nucleic acid molecules are hybridized to the combination for the purpose of defining a novel gene profile associated with that developmental stage, treatment, or disorder.

cDNAs and Their Use

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7) 215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2,6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically. cDNAs can be synthesized on a substrate. Synthesis on the surface of a substrate may be accomplished using a chemical coupling procedure and a piezoelectric printing apparatus as described by Baldeschweiler et al. (PCT publication WO95/25 1116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

The cDNAs may be used for a variety of purposes. For example, the combination of the invention may be used on an array. The array, in turn, can be used in high-throughput methods for detecting a related polynucleotide in a sample, screening a plurality of molecules or compounds to identify a ligand, diagnosing prostate cancer, or inhibiting or inactivating a therapeutically relevant gene related to the cDNA.

When the cDNAs of the invention are employed on an array, the cDNAs are arranged so that each cDNA is present at a specified location on the substrate. Because the cDNAs are at specified locations, the hybridization patterns and intensities, which together create a unique expression profile, can be interpreted in terms of expression levels of particular genes and can be correlated with a particular metabolic process, condition, disorder, disease, stage of disease, or treatment.

Hybridization

The cDNAs or fragments or complements thereof may be used in various hybridization technologies. The cDNAs may be labeled using a variety of reporter molecules by either PCR, recombinant, or enzymatic techniques. For example, a commercially available vector containing the cDNA is transcribed in the presence of an appropriate polymerase, such as 17 or SP6 polymerase, and at least one labeled nucleotide. Commercial kits are available for labeling and cleanup of such cDNAs. Radioactive (Amersham Biosciences (APB), Piscataway N.J.), fluorescent (Qiagen-Operon, Alameda Calif.), and chemiluminescent labeling (Promega, Madison Wis.) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide may be used to detect or quantify expression of a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1% SDS at either 45° C. (medium stringency) or 65⁰-68° C. (high stringency). At high stringency, hybridization complexes will remain stable only where the nucleic acids are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-6-4.9).

Dot-blot, slot-blot, low density and high density arrays are prepared and analyzed using methods known in the art. cDNAs from about 18 consecutive nucleotides to about 5000 consecutive nucleotides in length are contemplated by the invention and used in array technologies. Depending on the technology employed, the number of cDNAs on a substrate ranges from at least two to about 100,000. The high density array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. No. 5,605,662; and U.S. Pat. No. 5,958,342.)

Screening and Purification Assays

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of the polynucleotide in the biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a ligand, molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

Protein Production and Uses

The full length cDNAs or fragment thereof may be used to produce purified proteins using recombinant DNA technologies described herein and taught in Ausubel (sura; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.) and algorithms included in RasMol software (University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into a vector and transforming this vector into a host cell. The cloning vector used for the construction of cDNA libraries in the LIFESEQ databases (Incyte Genomics, Palo Alto Calif.) may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters includes SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the 431A peptide synthesizer (Applied Biosystems (ABI), Foster City Calif.). Proteins produced by any of the above methods may be used as pharmaceutical compositions to treat disorders associated with null or inadequate expression of the genomic sequence.

Screening and Purification Assays

A protein or a portion thereof produced using a cDNA of the invention may be used to screen a library or a plurality of molecules or compounds for a ligand with specific binding affinity or to purify a molecule or compound from a sample. The protein or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against a library or a plurality of ligands and the specificity of binding or formation of complexes between the expressed protein and the ligand may be measured. The ligands may be agonists, antagonists, antibodies, DNA molecules, enhancers, small drug molecules, immunoglobulins, inhibitors, mimetics, peptide nucleic acid molecules, peptides, pharmaceutical agents, proteins, and regulatory proteins, repressors, RNA molecules, ribozymes, and transcription factors or any other test molecule or compound that specifically binds the protein. An exemplary assay involves combining the mammalian protein or a portion thereof with the molecules or compounds under conditions that allow specific binding and detecting the bound protein to identify at least one ligand that specifically binds the protein.

This invention also contemplates the use of competitive drug screening assays in which neutralizing antibodies capable of binding the protein specifically compete with a test compound capable of binding to the protein or oligopeptide or fragment thereof. One method for high throughput screening using very small assay volumes and very small amounts of test compound is described in U.S. Pat. No. 5,876,946. Molecules or compounds identified by screening may be used in a model system to evaluate their toxicity, diagnostic, or therapeutic potential.

The protein may be used to purify a ligand from a sample. A method for using a protein to purify a ligand would involve combining the protein or a portion thereof with a sample under conditions to allow specific binding, recovering the bound protein, and using an appropriate chaotropic agent to separate the protein from the purified ligand.

Production of Antibodies

A protein encoded by a cDNA of the invention may be used to produce specific antibodies. Antibodies may be produced using an oligopeptide or a portion of the protein with inherent immunological activity. Methods for producing antibodies include: 1) injecting an animal, usually goats, rabbits, or mice, with the protein, or an antigenically-effective portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Labeling of Molecules for Assay

A wide variety of reporter molecules and conjugation techniques are known by those skilled in the art and may be used in various cDNA, polynucleotide, protein, peptide or antibody assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The cDNAs, or fragments thereof, may be used to detect and quantify differential gene expression; absence, presence, or excess expression of mRNAs; or to monitor mRNA levels during therapeutic intervention of prostate cancer. These cDNAs can also be utilized as markers of treatment efficacy against prostate cancer over a period ranging from several days to months. The diagnostic assay may use hybridization or amplification technology to compare gene expression in a biological sample from a patient to standard samples in order to detect differential expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a disorder associated with prostate cancer, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of proteins or cDNAs and a plurality of detectable complexes, wherein each complex is formed by specific binding between the protein or cDNA and a ligand in a in a sample. The cDNAs of the invention are used as elements on an array to analyze gene expression profiles. In one embodiment, the array is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

Two-dimensional polyacrylamide gel electrophoresis, mass spectrophotometry, western analysis, ELISA, RIA, fluorescent activated cell sorting (FACS), and protein or antibody arrays are used to produce protein expression profiles. Protocols for detecting and measuring protein expression using labeling moieties appropriate to the protocol are well known in the art.

Experimentally, expression profiles can also be evaluated by methods including, but not limited to, differential display by spatial immobilization or by gel electrophoresis, genome mismatch scanning, representational discriminant analysis, clustering, transcript imaging, and by protein or antibody arrays. Expression profiles produced by these methods may be used alone or in combination. The correspondence between mRNA and protein expression has been discussed by Zweiger (2001, Transducing the Genome. McGraw-Hill, San Francisco, Calif.) and Glavas et al. (2001; T cell activation upregulates cyclic nucleotide phosphodiesterases 8A1 and 7A3, Proc Natl Acad Sci 98:6319-6342) among others.

In another embodiment, animal models which mimic a human disease can be used to characterize expression profiles associated with a particular condition, disorder or disease; or treatment of the condition, disorder or disease. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety. Various immunoassays for proteins (also mentioned above) typically involve the formation of complexes between the protein and its specific antibody and the measurement of such complexes.

Antibody Arrays

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt et al. (2000) Nature Biotechnol 18:989-94).

Therapeutics

The cDNAs can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These sequences can be produced biologically by transforming an appropriate host cell with a vector containing the sequence of interest.

Molecules which regulate the activity of the cDNA or encoded protein are useful as therapeutics for treating prostate cancer. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells such as the mouse 129/SvJ cell line are placed in a blastocyst from the C57BLJ6 mouse strain, they resume normal development and contribute to tissues of the live-born animal. ES cells are preferred for use in the creation of experimental knockout and knockin animals. The method for this process is well known in the art and the steps are: the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

As described herein, the uses of the cDNAs, provided in the Sequence Listing of this application, and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art. It is also to be understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

I Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Invitrogen). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT plasmid (Stratagene), pSPORT1 plasmid (Invitrogen), or pINCY plasmid (Incyte Genomics). Recombinant plasmids were transformed into XL1-BLUE, XL1-BLUEMRF, or SOLR competent E. coli cells (Stratagene) or DH5a, DH10B, or ELECTROMAX DH10B competent E. coli cells (Invitrogen).

In some cases, libraries were superinfected with a 5×excess of the helper phage, M13K07, according to the method of Vieira et al. (1987, Methods Enzymol 153:3-11) and normalized or subtracted using a methodology adapted from Soares (1994, Proc Natl Acad Sci 91:9228-9232), Swaroop et al. (1991, Nucl Acids Res 19:1954), and Bonaldo et al. (1996, Genome Research 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares supra).

II Isolation and Sequencing of cDNA Clones

Plasmids were recovered from host cells by in vivo excision using the UNIZAP vector system (Stratagene) or by cell lysis. Plasmids were purified using one of the following: the Magic or WIZARD MINIPREPS DNA purification system (Promega); the AGTC MINIPREP purification kit (Edge BioSystems, Gaithersburg Md.); the QIAWELL 8, QIAWELL 8 Plus, or QIAWELL 8 Ultra plasmid purification systems, or the REAL PREP 96 plasmid purification kit (Qiagen). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland). cDNA sequencing reactions were processed using standard methods or high-throughput instrumentation such as the CATALYST 800 thermal cycler (ABI) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by APB or supplied in ABI sequencing kits such as the PRISM BIGDYE cycle sequencing kit (ABI). Electrophoretic separation of cDNA sequencing reactions and detection of labeled cDNAs were carried out using the MEGABACE 1000 DNA sequencing system (APB); the PRISM 373 or 377 sequencing systems (ABI) in conjunction with standard ABI protocols and base calling software; or other sequence analysis systems known in the art. Reading frames within the cDNA sequences were identified using standard methods (reviewed in Ausubel, supra, Unit 7.7).

III Extension of cDNA Sequences

Nucleic acid sequences were extended using the cDNA clones and oligonucleotide primers. One primer was synthesized to initiate 5′ extension of the known fragment, and the other, to initiate 3′ extension of the known fragment. The initial primers were designed using OLIGO software (Molecular Insights, Cascade Colo.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 mmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): 1: 94° C., 3 min; 2: 94° C., 15 sec; 3: 60° C., 1 min; 4: 68° C., 2 min; 5: 2, 3, and 4 repeated 20 times; 6: 68° C., 5 min; and 7: storage at 4° C. In the alternative, the parameters for primer pair T7 and SK+(Stratagene) were as follows: 1: 94° C., 3 min; 2: 94° C., 15 sec; 3: 57° C., 1 min; 4: 68° C., 2 min; 5: 2, 3, and 4 repeated 20 times; 6: 68° C., 5 min; and 7: storage at 4° C.

The concentration of DNA in each well was determined by dispensing 100 μl PICOGREEN reagent (0.25% reagent in 1×TE, v/v; Molecular Probes) and 0.5 μl of undiluted PCR product into each well of an opaque fluorimeter plate (Corning Costar, Acton Mass.) and allowing the DNA to bind to the reagent. The plate was scanned in a FLUOROSKAN II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC 18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transformed into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified by PCR using Taq DNA polymerase (APB) and Pfu DNA polymerase (Stratagene) with the following parameters: 1: 94° C., 3 min; 2: 94° C., 15 sec; 3: 60° C., 1 min; 4: 72° C., 2 min; 5: s 2, 3, and 4 repeated 29 times; 6: 72° C., 5 min; and 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

IV Assembly and Analysis of Sequences

The nucleic acid sequences presented in the Sequence Listing may contain occasional sequencing errors and unidentified nucleotides (N) that reflect state-of-the-art technology at the time the cDNA was first sequenced. Occasional sequencing errors and Ns may be resolved and SNPs verified either by resequencing the cDNA or using algorithms to compare the alignment of multiple sequences covering the region in which the N or potential SNP occurs. The sequences used in the verification may be identified from any available database using BLAST analysis and aligned using a variety of alignment algorithms described in Ausubel (supra, unit 7.7) and in Meyers (1995; Molecular Biology and Biotechnology, Wiley VCH, New York N.Y., pp. 856-853).

Component nucleotide sequences from chromatograms were subjected to PHRED analysis (Phil Green, University of Wash., Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417-424).

Processed sequences were subjected to assembly procedures in which the sequences were assigned to bins, one sequence per bin. Sequences in each bin were assembled to produce consensus sequences, templates. Subsequent new sequences were added to existing bins using BLAST (Altschul (supra); Altschul et al. (supra); Karlin et al. (1988) Proc Natl Acad Sci 85:841-845), BLASTn (vers.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil Green, supra). Bins with several overlapping component sequences were assembled using DEEP PHRAP (Phil Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Genomics).

The assembled templates were annotated using the following procedure. Template sequences were analyzed using BLASTn (vers. 2.0, NCBI) versus GBpri (GenBank vers. 116). “Hits” were defined as an exact match having from 95% local identity over 200 base pairs through 100% local identity over 100 base pairs, or a homolog match having an E-value equal to or greater than 1×10⁻⁸. (The “E-value” quantifies the statistical probability that a match between two sequences occurred by chance). The hits were subjected to frameshift FASTx versus GENPEPT (GenBank version 109). In this analysis, a homolog match was defined as having an E-value of 1×10⁻⁸. The assembly method used above was described in U.S. Ser. No. 09/276,534, filed Mar. 25, 1999, and the LIFESEQ GOLD user manual (Incyte Genomics).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in U.S. Ser. No. 08/812,290, filed Mar. 6, 1997; U.S. Ser. No. 08/947,845, filed Oct. 9, 1997; U.S. Pat. No. 5,953,727; and U.S. Ser. No. 09/034,807, filed Mar. 4, 1998. Template sequences may be further queried against public databases such as the GenBank rodent, mammalian, vertebrate, eukaryote, prokaryote, and human EST databases.

V Selection of Sequences, Microarray Preparation and Use

Incyte clones represent template sequences derived from the LIESEQ GOLD assembled human sequence database (Incyte Genomics). In cases where more than one clone was available for a particular template, the 5′-most clone in the template was used on the microarray. The HUMAN GENOME GEM series 1-3 microarrays (Incyte Genomics) contain 28,626 array elements which represent 10,068 annotated clusters and 18,558 unannotated clusters.

For the UNIGEM series microarrays (Incyte Genomics), Incyte clones were mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) was chosen, verified, and used in the construction of the microarray. The UNIGEM V microarray (Incyte Genomics) contains 7075 array elements which represent 4610 annotated genes and 2,184 unannotated clusters.

To construct microarrays, cDNAs were amplified from bacterial cells using primers complementary to vector sequences flanking the cDNA insert. Thirty cycles of PCR increased the initial quantity of cDNA from 1-2 ng to a final quantity greater than 5 μg. Amplified cDNAs were then purified using SEPHACRYL-400 columns (APB). Purified cDNAs were immobilized on polymer-coated glass slides. Glass microscope slides (Corning, Corning N.Y.) were cleaned by ultrasound in 0.1% SDS and acetone, with extensive distilled water washes between and after treatments. Glass slides were etched in 4% hydrofluoric acid (VWR Scientific Products, West Chester Pa.), washed extensively in distilled water, and coated with 0.05% aminopropyl silane (Sigma Aldrich, St Louis Mo.) in 95% ethanol. Coated slides were cured in a 110° C. oven. cDNAs were applied to the coated glass substrate using a procedure described in U.S. Pat. No. 5,807,522. One microliter of the cDNA at an average concentration of 100 ng/ul was loaded into the open capillary printing element by a high-speed robotic apparatus which then deposited about 5 nl of cDNA per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

Twenty-two UNIGEMV arrays were used to evaluate differential expression across experimental and control samples.

VI Preparation of Samples

Cell Growth and Treatments

The following cell lines were obtained from ATCC (Manassus Va.) and cultured in media according to the manufacturer's protocols: PrEC is a primary prostate epithelial cell line isolated from a normal donor; PC-3 is a prostate adenocarcinoma cell line isolated from a 62 year-old male with grade IV prostate adenocarcinoma metastasized to the bone; DU-145 is a prostate carcinoma cell line isolated from a 69 year-old man with widespread metastatic disease. DU-145 was isolated from a brain metastasis and has no detectable hormone sensitivity; LNCaP is a prostate carcinoma cell line isolated from a lymph node biopsy of a 50 year-old male with metastatic prostate carcinoma. LNCaP cells are responsive to 5-alpha-dihydrotestosterone and express androgen receptors. All cell cultures were incubated in low serum media 48 hours prior to treatment.

Cells were subjected to the following treatments: R1881 Androgen; PrEC and LNCaP cells were treated with 5 nM R1881 Androgen (Methyltrienolone) for 8, 14, 24, and 38 hrs. EGF; PrEC, LNCap, DU-145, and PC-3 cells were treated with 50 ng/ml of EGF for 4, 8, 14, 24, and 38 hrs. FGF; PrEC, DU-145, and PC-3 cells were treated with 50 ng/ml of FGF for 4, 8, 14, 24, and 38 hrs. TGF-α; PrEC and PC-3 cells were treated with 50 ng/ml of TGF-α for 8, 14, 24, and 36 hrs. TGF-β; PrEC and DU-145 cells were treated with 5 ng/ml of TGF-β for 4, 8, 14, 24, and 38 hrs. Cells were harvested at each time point and prepared as described below.

Isolation and Labeling of Sample Polynucleotides

Cells were harvested and lysed in 1 ml of TRIZOL reagent (5×10⁶ cells/ml; Invitrogen). The lysates were vortexed thoroughly and incubated at room temperature for 2-3 minutes and extracted with 0.5 ml chloroform. The extract was mixed, incubated at room temperature for 5 minutes, and centrifuged at 15,000 rpm for 15 minutes at 4° C. The aqueous layer was collected and an equal volume of isopropanol was added. Samples were mixed, incubated at room temperature for 10 minutes, and centrifuged at 15,000 rpm for 20 minutes at 4° C. The supernatant was removed, and the RNA pellet was washed with 1 ml of 70% ethanol, centrifuged at 15,000 rpm at 4° C., and resuspended in RNAse-free water. The concentration of the RNA was determined by measuring the optical density at 260 nm.

Poly(A) RNA was prepared using an OLIGOTEX mRNA kit (Qiagen) with the following modifications: OLIGOTEX beads were washed in tubes instead of on spin columns, resuspended in elution buffer, and then loaded onto spin columns to recover mRNA. To obtain maximum yield, the mRNA was eluted twice.

Each poly(A) RNA sample was reverse transcribed using MMLV reverse-transcriptase, 0.05 pg/μl oligo-d(T) primer (21mer), 1×first strand buffer, 0.03 units/ul RNAse inhibitor, 500 uM dATP, 500 uM dGTP, 500 uM dTTP, 40 uM dCTP, and 40 uM either dCTP-Cy3 or dCTP-CyS (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

cDNAs were purified using two successive CHROMA SPIN 30 gel filtration spin columns (Clontech). Cy3- and CyS-labeled reaction samples were combined as described below and ethanol precipitated using 1 ml of glycogen (1 mg/ml), 60 ml sodium acetate, and 300 ml of 100% ethanol. The cDNAs were then dried to completion using a SpeedVAC system (Savant Instruments, Holbrook N.Y.) and resuspended in 14 μl 5×SSC/0.2% SDS.

VII Hybridization and Detection

Hybridization reactions contained 9 μl of sample mixture containing 0.2 μg each of Cy3 and Cy5 labeled cDNA synthesis products in 5×SSC, 0.2% SDS hybridization buffer. The mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5×SSC in a corner of the chamber. The chamber containing the microarrays was incubated for about 6.5 hours at 60° C. The microarrays were washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light was focused on the microarray using a 20×microscope objective (Nikon, Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Genomics). Significance was defined as signal to background ratio exceeding 2×and area hybridization exceeding 40%.

VIII Data Analysis and Results

Array elements that exhibited at least a two-fold change in expression at one or more time points, a signal intensity over 250 units, a signal-to-background ratio of at least 2.5, and an element spot size of at least 40% were identified as differentially expressed using the GEMTOOLS program (Incyte Genomics). Each of the treatment groups for the prostate tumor cell lines, LNCaP, DU-145, and PC-3 were compared with the untreated cell line, and with normal PrEC cells similarly treated. The cDNAs that are differentially expressed are shown in Table 1 and are divided into treatment groups, and by up- or down-regulated expression. The cDNAs are identified by their SEQ ID NO and TEMPLATE ID, and by the description associated with at least a fragment of a polynucleotide found in GenBank. The descriptions were obtained using the sequences of the Sequence Listing and BLAST analysis.

IX Other Hybridization Technologies and Analyses

Other hybridization technologies utilize a variety of substrates such as nylon membranes, capillary tubes, etc. Arranging cDNAs on polymer coated slides is described in EXAMPLE V; sample cDNA preparation and hybridization and analysis using polymer coated slides is described in EXAMPLES VI and VII, respectively.

The cDNAs are applied to a membrane substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100° C. for five min, and briefly centrifuging. The denatured cDNA is then added to a REDIPRIME tube (APB), gently mixed until blue color is evenly distributed, and briefly centrifuged. Five microliters of [³²P]dCTP is added to the tube, and the contents are incubated at 37° C. for 10 min. The labeling reaction is stopped by adding 5,ul of 0.2M EDTA, and probe is purified from unincorporated nucleotides using a PROBEQUANT G-50 microcolumn (APB). The purified probe is heated to 100° C. for five min, snap cooled for two min on ice.

Membranes are pre-hybridized in hybridization solution containing 1% Sarkosyl and 1×high phosphate buffer (0.5 M NaCl, 0.1 M Na₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined.

X Further Characterization of Differentially Expressed cDNAs and Proteins

Clones were compared with the sequences in the LIFESEQ Gold 5.1 database (Incyte Genomics) using BLAST analysis, and an Incyte template and its variants were chosen for each clone. The template and variants were compared with the sequences in the GenBank database using BLAST analysis to acquire annotation. The nucleotide sequences were translated into amino acid sequence which was compared against the sequences in the GENPEPT and other protein databases using BLAST analysis to acquire annotation and other characterization such as domains and structural and functional motifs.

Percent sequence identity can also be determined electronically for two or more amino acid or nucleic acid sequences using the MEGALIGN program of LASERGENE software (DNASTAR). The percent similarity between two amino acid sequences is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity.

Sequences with conserved protein motifs may be searched using the BLOCKS search program. This program analyses sequence information contained in the Swiss-Prot and PROSITE databases and is useful for determining the classification of uncharacterized proteins translated from genomic or cDNA sequences (Bairoch.(supra); Attwood (supra). PROSITE database is a useful source for identifying functional or structural domains that are not detected using motifs due to extreme sequence divergence. Using weight matrices, these domains are calibrated against the SWISS-PROT database to obtain a measure of the chance distribution of the matches.

The PRINTS database can be searched using the BLIMPS search program to obtain protein family “fingerprints”. The PRINTS database complements the PROSITE database by exploiting groups of conserved motifs within sequence alignments to build characteristic signatures of different protein families. For both BLOCKS and PRINTS analyses, the cutoff scores for local similarity were: >1300=strong, 1000-1300=suggestive; for global similarity were: p<exp-3; and for strength (degree of correlation) were: >1300=strong, 1000-1300=weak.

XI Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with IPTG. Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XII Production of Specific Antibodies

A denatured protein from a reverse phase HPLC separation is obtained in quantities up to 75 mg. This denatured protein is used to immunize mice or rabbits following standard protocols. About 100 Itg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to determine regions of high antigenicity, essentially antigenically-effective epitopes of the protein. The optimal sequences for immunization are usually at the C-terminus, the N-terminus, and those intervening, hydrophilic regions of the protein that are likely to be exposed to the external environment when the protein is in its natural conformation. Typically, oligopeptides about 15 residues in length are synthesized using an 431 Peptide synthesizer (ABI) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XIII Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA or fragments thereof and the protein or portions thereof are labeled with ³²P-dCTP, Cy3-dCTP, Cy5-dCTP (APB), or BIODIPY or FITC (Molecular Probes), respectively. Candidate molecules or compounds previously arranged on a substrate are incubated in the presence of labeled nucleic or amino acid. After incubation under conditions for either a cDNA or a protein, the substrate is washed, and any position on the substrate retaining label, which indicates specific binding or complex formation, is assayed. The binding molecule is identified by its arrayed position on the substrate. Data obtained using different concentrations of the nucleic acid or protein are used to calculate affinity between the labeled nucleic acid or protein and the bound molecule. High throughput screening using very small assay volumes and very small amounts of test compound is fully described in U.S. Pat. No. 5,876,946, incorporated by reference herein.

All patents and publications mentioned in the specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| | | | | | |
|---|---|---|---|---|---|
| SEQ | | | | | |
| ID | TEMPLATE | GI | | | Cell |
| N0 | ID | Number | E-Value | Annotation | Line |

Androgen treated, downregulated

| | | | | | |
|---|---|---|---|---|---|
| 1 | 441269.2 | g2895090 | 0 | Human RalBP1-interacting protein (POB1) mRNA, complete cds. | LNCaP |
| 2 | 3161.5 | g6996441 | 0 | Human CTL1 gene. | LNCaP |
| 3 | 977955.7 | g438372 | 0 | Human mRNA for protein kinase C mu. | LNCaP |
| 4 | 350754.2 | g307503 | 0 | Human transglutaminase E3 (TGASE3) mRNA, complete cds. | LNCaP |
| 5 | 235191.4 | g2062372 | 0 | Human cyclin-selective ubiquitin carrier protein mRNA, complete cds. | LNCaP |
| 6 | 1099593.13 | g1702923 | 0 | Human mRNA for p0071 protein. | LNCaP |
| 7 | 1099593.2 | g1702923 | 0 | Human mRNA for p0071 protein. | LNCaP |
| 8 | 003161.7c | g6996441 | 0 | Human CTL1 gene. | LNCaP |
| 9 | 415650.5 | g190663 | 0 | Human prostate-specific membrane antigen (PSM) mRNA, complete cds. | LNCaP |
| 10 | 204401.1 | g2970122 | 0 | Human prostate-specific membrane antigen (PSM) gene, complete cds. | LNCaP |
| 11 | 347915.14 | g1503997 | 0 | Human mRNA for KIAA0207 gene, complete cds. | LNCaP |
| 12 | 997395.1 | g307513 | 0 | Human transducin-like enhancer protein (TLE3) mRNA, complete cds. | LNCaP |
| 13 | 332783.1 | g2795902 | 0 | Human clone 23860 mRNA sequence. | LNCaP |
| 14 | 422289.1 | | | Incyte Unique | LNCaP |
| 15 | 899410.5 | g4165324 | 0 | Human plasma membrane calcium ATPase isoform 1 (ATP2B1) gene, alternative | LNCaP |
| | | | | splice products, partial cds. | |
| 16 | 404155.2 | g1255601 | 0 | Human mRNA for cGMP-dependent protein kinase type I alpha, complete cds. | LNCaP |
| 17 | 412065.22 | g1374791 | 0 | Human selenium-binding protein (hSBP) mRNA, complete cds. | LNCaP |
| 18 | 412065.2 | g1374791 | 7.00E−92 | Human selenium-binding protein (hSBP) mRNA, complete cds. | LNCaP |
| 19 | 345860.21 | g29709 | 0 | Human mRNA for cathepsin H (EC 3.4.22.16). | LNCaP |
| 20 | 199101.1 | g1296629 | 0 | Human mRNA for UDP-GalNAc: polypeptide N-acetylgalactosaminyl transferase | LNCaP |
| | | | | (GalNAc-T3). | |
| 21 | 196606.8c | g2924334 | 0 | Human mRNA for exportin (tRNA). | LNCaP |
| 22 | 475146.3 | g619876 | 0 | Human mRNA for 3-hydroxy-3-methylglutaryl CoA synthase. | LNCaP |
| 23 | 474069.8 | g7020654 | 0 | unnamed protein product [Homo sapiens] | LNCaP |
| 24 | 255115.2 | g5881245 | 0 | Human UDP-glucuronosyltransferase 2B15 (UGT2B15) mRNA, UGT2B15-Y85 | LNCaP |
| | | | | allele, complete cds. | |
| 25 | 255115.4 | g3287472 | 0 | Human C19steroid specific UDP-glucuronosyltransferase mRNA, complete cds. | LNCaP |
| 26 | 216331.1 | | | Incyte Unique | LNCaP |
| 27 | 482517.3 | g6721135 | 0 | Homo sapiens chromosome 14 clone CTD-2547F10, complete | LNCaP |
| 28 | 480653.1 | g6841247 | 0 | Human HSPC299 mRNA, partial cds. | LNCaP |
| 29 | 978047.1 | g2852631 | 0 | Human clone 23649 and 23755 unknown mRNA, partial cds. | LNCaP |
| 30 | 330977.1c | g183784 | 0 | Human androgen receptor (hAR) gene sequence. | LNCaP |
| 31 | 343502.9 | g3779225 | 0 | Human secreted cement gland protein XAG-2 homolog (hAG-2/R) mRNA, | LNCaP |
| | | | | complete cds. | |
| 32 | 903104.11c | g181122 | 0 | Human cleavage signal 1 protein mRNA, complete cds. | LNCaP |
| 33 | 898068.6 | g6714698 | 0 | Human mRNA for sugar transporter (SLC2A6 gene). | LNCaP |
| 34 | 903104.1 | g181122 | 5.00E−55 | Human cleavage signal 1 protein mRNA, complete cds. | LNCaP |
| 35 | 903104.8 | g181122 | 0 | Human cleavage signal 1 protein mRNA, complete cds. | LNCaP |
| 36 | 412065.21 | g1374791 | 0 | Human selenium-binding protein (hSBP) mRNA, complete cds. | LNCaP |
| 37 | 399067.1 | g3157804 | 6.60E−257 | neuronal leucine-rich repeat protein | LNCaP |
| 38 | 237709.11 | g6172220 | 0 | Human SPON2 mRNA for spondin 2, complete cds. | LNCaP |
| 39 | 237709.5 | g6172220 | 0 | Human SPON2 mRNA for spondin 2, complete cds. | LNCaP |
| 40 | 216437.4 | | | Incyte Unique | LNCaP |
| 41 | 255824.52 | g178350 | 0 | Human aldolase A mRNA, complete cds. | LNCaP |
| 42 | 27798.1 | g6453517 | 0 | Human mRNA; cDNA DKFZp434M1317 (from clone DKFZp434M1317). | LNCaP |
| 43 | 60671.7 | g3043675 | 0 | Human mRNA for KIAA0576 protein, partial cds. | LNCaP |
| 44 | 376085.9 | g6453582 | 0 | Human mRNA; cDNA DKFZp434G1221 (from clone DKFZp434G1221). | LNCaP |
| 45 | 404467.1 | g4826465 | 1.20E−56 | dJ287G14.2 (PUTATIVE novel seven transmembrane domain protein) | LNCaP |
| 46 | 903508.12 | g5262490 | 0 | Human mRNA; cDNA DKFZp564D0462 (from clone DKFZp564D0462). | LNCaP |
| 47 | 334387.1 | g632497 | 0 | Human cleavage stimulation factor 77 kDa subunit mRNA, complete cds. | LNCaP |
| 48 | 290403.5 | g6808173 | 0 | Human mRNA; cDNA DKFZp564M1178 (from clone DKFZp564M1178); partial | LNCaP |
| 49 | 391406.5 | g187701 | 0 | Human MHC protein homologous to chicken B complex protein mRNA, complete | LNCaP |
| 50 | 391406.24 | g187701 | 0 | Human MHC protein homologous to chicken B complex protein mRNA, complete | LNCaP |
| 51 | 474588.21 | g339700 | 0 | Human polyadenylate binding protein (TIA-1) mRNA, complete cds. | LNCaP |
| 52 | 124541.1 | | | Incyte Unique | LNCaP |
| 53 | 246862.9 | g28937 | 0 | Human mRNA for mitochondrial ATP synthase (F1-ATPase) alpha subunit. | LNCaP |
| 54 | 246862.17 | g28937 | 0 | Human mRNA for mitochondrial ATP synthase (F1-ATPase) alpha subunit. | LNCaP |
| 55 | 13040.1 | | | Incyte Unique | LNCaP |
| 56 | 236480.3 | g1556398 | 0 | Human mRNA for FAN protein. | LNCaP |

Androgen treated, upregulated

| | | | | | |
|---|---|---|---|---|---|
| 57 | 314831.5 | g31920 | 0 | Human GST1-Hs mRNA for GTP-binding protein. | LNCaP |
| 58 | 391940.1 | g32451 | 0 | Human pHS1-2 mRNA with ORF homologous to membrane receptor proteins. | LNCaP |
| 59 | 454958.13 | g182482 | 0 | Human fibroblast collagenase inhibitor mRNA, complete cds. | LNCaP |
| 60 | 1090481.2c | g189152 | 2.00E−12 | Human oligodendrocyte myelin glycoprotein (OMG) exons 1-2; | LNCaP |
| | | | | neurofibromatosis 1 (NF1) exons 28-49; ecotropic viral integration site 2B | |
| | | | | (EVI2B) exons 1-2; ecotropic viral integration site 2A (EVI2A) exons 1-2; | |
| 61 | 238203.11 | g340236 | 0 | Human vinculin mRNA, complete cds. | LNCaP |
| 62 | 994057.21 | g531475 | 0 | Human PPP1CB mRNA. | LNCaP |
| 63 | 346730.5 | g2463627 | 0 | Human putative monocarboxylate transporter (MCT) mRNA, complete cds. | LNCaP |
| 64 | 346730.2c | g2463627 | 0 | Human putative monocarboxylate transporter (MCT) mRNA, complete cds. | LNCaP |
| 65 | 458903.1 | g34055 | 7.00E−14 | Human K7 gene for simple epithelial cell keratin K7 (exon 4). | LNCaP |
| 66 | 482336.2 | g34067 | 0 | Human mRNA fragment for mesothelial type II keratin K7. | LNCaP |
| 67 | 482336.14 | g34067 | 0 | Human mRNA fragment for mesothelial type II keratin K7. | LNCaP |
| 68 | 66522.1 | | | Incyte Unique | LNCaP |
| 69 | 300294.3 | g1256819 | 0 | Human signal recognition particle (SRP54) mRNA, complete cds. | LNCaP |
| 70 | 474372.8 | g6807723 | 0 | Human mRNA; cDNA DKFZp434J1114 (from clone DKFZp434J1114); partial | LNCaP |
| 71 | 475028.7 | g575271 | 0 | Human SPHAR gene for cyclin-related protein. | LNCaP |
| 72 | 481223.3 | g2909359 | 0 | Human mRNA for Sox10 protein. | LNCaP |
| 73 | 332499.1 | g7022927 | 9.00E−38 | unnamed protein product [Homo sapiens] | LNCaP |
| 74 | 234340.7 | g4007417 | 0 | Human Ets transcription factor PDEF (PDEF) mRNA, complete cds. | LNCaP |
| 75 | 234340.15c | g6721497 | 0 | Human PSE mRNA for prostate ets, complete cds. | LNCaP |
| 76 | 255778.11 | g1373172 | 0 | Human NADH: ubiquinone oxidoreductase subunit B13 (B13) mRNA, complete | LNCaP |
| 77 | 317586.1 | g3265061 | 0 | Human N-acetyltransferase-1 (NAT1) gene, NAT1*26B allele, complete cds. | LNCaP |
| 78 | 1093574.1c | g3335147 | 0 | Human short form transcription factor C-MAF (c-maf) mRNA, complete cds. | LNCaP |
| 79 | 355658.1 | | | Incyte Unique | LNCaP |
| 80 | 201342.4 | g5199315 | 0 | Human non-ocogenic Rho GTPase-specific GTP exchange factor (proto-LBC) | LNCaP |
| | | | | mRNA, complete cds. | |
| 81 | 204542.1 | g6425039 | 0 | Human N-acetylglucosamine-1-phosphodiester alpha-N-acetylglucosaminidase | LNCaP |
| | | | | mRNA, complete cds. | |
| 82 | 428742.1 | | | Incyte Unique | LNCaP |
| 83 | 204386.1 | g7020228 | 1.00E−128 | unnamed protein product [Homo sapiens] | LNCaP |
| 84 | 335086.1 | g1543067 | 0 | Human Has2 mRNA, complete cds. | LNCaP |
| 85 | 228302.1 | | | Incyte Unique | LNCaP |
| 86 | 1063057.1 | g488552 | 0 | Human zinc finger protein ZNF134 mRNA, complete cds. | LNCaP |

EGF treated, downregulated

| | | | | | |
|---|---|---|---|---|---|
| 87 | 199905.1 | g3449309 | 0 | Human mRNA for MEGF9, partial cds. | DU145 |
| 88 | 11329.1 | g4835609 | 0 | Homo sapiens genomic DNA, chromosome 21q22.1, D21S226-AML | LNCaP |
| 89 | 237536.18 | g4165090 | 0 | Human NADH-ubiquinone oxidoreductase PDSW subunit mRNA, complete cds. | DU145 |
| 90 | 330878.6 | g5565654 | 0 | Human cullin 4A (CUL4A) mRNA, complete cds. | DU145 |
| 91 | 331793.11 | g5262498 | 0 | Human mRNA; cDNA DKFZp564G2362 (from clone DKFZp564G2362). | DU145 |
| 92 | 902895.2 | g4003380 | 5.00E−09 | Human genomic DNA of 8p21.3-p22 anti-oncogene of hepatocellular colorectal | DU145 |
| | | | | and non-small cell lung cancer, segment 3/11. | |
| 93 | 7273.1 | g5918013 | 0 | Human DNA sequence from clone 423B22 on chromosome 1p33-35.3, | PC3 |
| 94 | 190771.2 | g5911950 | 0 | Human mRNA; cDNA DKFZp727G051 (from clone DKFZp727G051); partial | DU145 |
| 95 | 1095702.14 | g187282 | 0 | Human cation-dependent mannose 6-phosphate-specific receptor mRNA, | DU145 |
| | | | | complete cds. | |
| 96 | 1095702.4c | g187282 | 0 | Human cation-dependent mannose 6-phosphate-specific receptor mRNA, | DU145 |
| | | | | complete cds. | |
| 97 | 977667.1 | g179699 | 0 | Human C5a anaphylatoxin receptor mRNA, complete cds. | DU145 |
| 98 | 253534.14 | g5410450 | 0 | Human interferon-induced protein p78 (MX1) gene, complete cds. | PC3 |
| 99 | 1136709.5 | g187273 | 0 | Human eosinophil Charcot-Leyden crystal (CLC) protein (lysophospholipase) | PC3 |
| | | | | mRNA, complete cds. | |
| 100 | 1136709.6 | g187273 | 0 | Human eosinophil Charcot-Leyden crystal (CLC) protein (lysophospholipase) | PC3 |
| | | | | mRNA, complete cds. | |
| 101 | 382293.16 | g3676496 | 0 | Human inducible 6-phosphofructo-2-kinase/fructose 2,6-bisphosphatase (IPFK-2) | DU145 |
| | | | | mRNA, complete cds. | |
| 102 | 382293.15 | g29542 | 0 | Human mRNA for complement component Cls. | DU145 |
| 103 | 235943.39c | g37053 | 0 | Human mRNA for transmembrane epithelial tumour mucin antigen. | DU145 |
| 104 | 235943.36 | g2055365 | 0 | Human polymorphic epithelial mucin (PEM) gene, complete cds. | DU145 |
| 105 | 401434.1 | g4914611 | 0 | Human mRNA; cDNA DKFZp586B2023 (from clone DKFZp586B2023). | DU145 |
| 106 | 234107.2 | g603559 | 0 | Human LU gene for Lutheran blood group glycoprotein. | DU145 |
| 107 | 233660.2c | g3879501 | 1.10E−41 | similar to ubiquitin carboxyl-terminal hydrolase; cDNA EST EMBL: D33366 | DU145 |
| | | | | comes from this gene; cDNA EST EMBL: D33965 comes from this gene; cDNA | |
| | | | | EST EMBL: D33822 comes from this gene; cDNA EST EMBL: D34547 comes | |
| | | | | from this gene; cDNA EST EMBL: D37684 | |
| 108 | 482423.1 | g2808656 | 0 | Human complete genomic sequence between D16S3070 and D16S3275, | DU145 |
| | | | | containing Familial Mediterranean Fever gene disease. | |
| 109 | 441298.14 | g3327137 | 0 | Human mRNA for KIAA0662 protein, partial cds. | DU145 |
| 110 | 235333.1 | | | Incyte Unique | DU145 |
| 111 | 221872.11c | g5771534 | 0 | Human secreted protein of unknown function (SPUF), mRNA, complete cds. | DU145 |
| 112 | 992317.12 | | | Incyte Unique | DU145 |
| 113 | 230936.6 | g4929618 | 0 | Human CGI-75 protein mRNA, complete cds. | DU145 |
| 114 | 029508.1c | | | | DU145 |
| 115 | 984900.1 | g3881473 | 8.7 | ZK1037.11 [Caenorhabditis elegans] | DU145 |
| 116 | 405158.1 | g6807861 | 0 | Human mRNA; cDNA DKFZp434A0225 (from clone DKFZp434A0225). | DU145 |
| 117 | 334177.1 | | | Incyte Unique | DU145 |
| 118 | 480187.81 | g338928 | 0 | Human T-cell receptor active beta-chain V-D-J-beta-1.2-C-beta-1 (TCRB) | DU145 |
| | | | | mRNA, partial cds. | |
| 119 | 62042.4 | g6808161 | 0 | Human mRNA; cDNA DKFZp761O051 (from clone DKFZp761O051). | DU145 |
| 120 | 1087696.7 | g4929632 | 0 | Human CGI-82 protein mRNA, complete cds. | DU145 |
| 121 | 259805.28 | g189511 | 0 | Human protein p78 mRNA, complete cds. | DU145 |
| 122 | 399128.1 | | | Incyte Unique | DU145 |
| 123 | 986565.15 | g5514676 | 0 | Human mRNA for thiamine transporter (THTR-1), partial. | DU145 |
| 124 | 978378.3 | g4001636 | 5.00E−41 | Human DNA, anonymous heat-stable fragment RP8-8. | DU145 |
| 125 | 979575.2c | | | | DU145 |
| 126 | 233594.4 | g6721505 | 0.85 | hypothetical protein [Oryza sativa] | DU145 |
| 127 | 238635.1 | g219534 | 0 | Human CGM1a mRNA for CD66d. | DU145 |
| 128 | 1080496.1 | g4500232 | 0 | Human mRNA; cDNA DKFZp586B1122 (from clone DKFZp586B1122). | DU145 |
| 129 | 351204.2 | g35687 | 0 | Human mRNA for protease 3. | DU145 |
| 130 | 235394.5 | | | Incyte Unique | DU145 |
| 131 | 403242.1 | g5725470 | 0 | Human mRNA full length insert cDNA clone EUROIMAGE 1035904. | DU145 |
| 132 | 427529.9 | g4884083 | 0 | Human mRNA; cDNA DKFZp564O243 (from clone DKFZp564O243); partial | DU145 |
| 133 | 474868.2 | g3603460 | 0 | Human heat shock protein hsp40-3 mRNA, complete cds. | DU145 |
| 134 | 1095839.17 | g4929742 | 0 | Human CGI-137 protein mRNA, complete cds. | DU145 |
| 135 | 1095839.1 | g4050043 | 0 | Human RAD17 isoform 4 (RAD17) mRNA, complete cds. | DU145 |
| 136 | 244251.4 | | | Incyte Unique | DU145 |
| 137 | 21555.1 | | | Incyte Unique | DU145 |
| 138 | 234157.3 | g6716409 | 0 | Human chromosome 16 open reading frame 5 (C16orf5) mRNA, complete cds. | LNCaP |
| 139 | 903478.1 | | | Incyte Unique | LNCaP |
| 140 | 411296.2 | g183911 | 0 | Human hemopoietic cell protein-tyrosine kinase (HCK) gene, complete cds, clone | DU145 |
| | | | | lambda-a2/1a. | |
| 141 | 18044.1 | g4929732 | 1.00E−15 | Human CGI-132 protein mRNA, complete cds. | DU145 |
| 142 | 407260.2 | g7022708 | 0 | unnamed protein product [Homo sapiens] | PC3 |
| 143 | 978765.2 | | | Incyte Unique | DU145 |
| 144 | 338274.1 | | | Incyte Unique | DU145 |
| 145 | 231270.1 | g829258 | 3.00E−04 | Chitinase [Beta vulgaris] | DU145 |
| 146 | 18092.1 | | | Incyte Unique | DU145 |
| 147 | 404197.8c | | | | DU145 |
| 148 | 404197.4 | g3746548 | 0 | Human cyclin K (CPR4) mRNA, complete cds. | DU145 |
| 149 | 5460.1 | g4914518 | 0 | Human DNA sequence from clone CTA-216E10 on chromosome 22 | DU145 |
| 150 | 346511.4 | g338651 | 0 | Human 69 kDa 2′5′ oligoadenylate synthetase (P69 2-5A synthetase) mRNA, | PC3 |
| | | | | complete cds. | |
| 151 | 346511.6 | g338653 | 0 | Human 71 kDa 2′5′ oligoadenylate synthetase (p69 2-5A synthetase) mRNA, | PC3 |
| | | | | complete cds. | |
| 152 | 331233.2 | g437379 | 0 | Human potassium voltage-gated channel (KCNC1) mRNA. | DU145 |
| 153 | 234056.5 | g6164748 | 0 | Human F-box protein Fbx23 (FBX23) mRNA, partial cds. | DU145 |

EGF treated, upregulated

| | | | | | |
|---|---|---|---|---|---|
| 154 | 238118.1 | g4558635 | 0 | Homo sapiens chromosome 19, BAC 82621 (CIT-B-139a18), complete | PC3 |
| 155 | 233596.5 | g7209311 | 2.00E−40 | FLJ00005 protein [Homo sapiens] | PC3 |
| 156 | 988653.1 | g31129 | 0 | Human mRNA for early growth response protein 1 (hEGR1). | LNCaP |
| 157 | 470468.26 | g1050524 | 0 | Human mRNA for uridine phosphorylase. | DU145 |
| 158 | 470468.25 | g1050524 | 0 | Human mRNA for uridine phosphorylase. | DU145 |
| 159 | 903479.3 | g3882330 | 0 | Human mRNA for KIAA0805 protein, partial cds. | PC3 |
| 160 | 267918.1 | | | Incyte Unique | PC3 |
| 161 | 1094412.1 | g1143491 | 0 | Human mRNA for BiP protein. | DU145 |
| 162 | 1094107.1 | g1143491 | 0 | Human mRNA for BiP protein. | DU145 |
| 163 | 218452.4 | g575265 | 0 | Human PPP1R3 mRNA for protein phosphatase 1, glycogen-binding regulatory | PC3 |
| | | | | subunit. | |

FGF treated, downregulated

| | | | | | |
|---|---|---|---|---|---|
| 164 | 12178.2 | | | Incyte Unique | DU145 |
| 165 | 100653.3 | g436999 | 0 | Human HRY gene, complete cds. | PC3 |
| 166 | 978478.4 | | | Incyte Unique | PC3 |
| 167 | 85942.2 | | | Incyte Unique | PC3 |
| 168 | 85942.3 | | | Incyte Unique | PC3 |
| 169 | 977470.16c | g392426 | 0 | Human zinc-finger protein (bcl-6) mRNA, complete cds. | PC3 & DU14 |
| 170 | 283762.2 | g1469867 | 0 | Human mRNA for KIAA0143 gene, partial cds. | PC3 |
| 171 | 331065.2 | g537293 | 0 | Human negative regulator of programmed cell death ICH-1S (Ich-1) mRNA, | PC3 |
| | | | | complete cds. | |
| 172 | 331065.5 | g537291 | 0 | Human positive regulator of programmed cell death ICH-1L (Ich-1) mRNA, | PC3 |
| | | | | complete cds. | |
| 173 | 252782.4 | g6103638 | 0 | Human F-box protein FBL5 mRNA, partial cds. | PC3 |
| 174 | 229068.1 | g2687858 | 4.00E−62 | renal organic anion transporter [Pseudopleuronectes americanus] | DU145 |
| 175 | 407450.7 | g1066391 | 0 | Human t(3; 5)(q25.1; p34) fusion gene NPM-MLF1 mRNA, complete cds. | DU145 |
| 176 | 200163.2 | g5817262 | 0 | Human mRNA; cDNA DKFZp566C224 (from clone DKFZp566C224). | DU145 |
| 177 | 200163.12c | g5817262 | 0 | Human mRNA; cDNA DKFZp566C224 (from clone DKFZp566C224). | DU145 |
| 178 | 1328237.4 | g453578 | 0 | Human mRNA for proto-oncogene protein, complete cds. | PC3 |
| 179 | 333965.1 | g1695872 | 0 | Human ser-thr protein kinase PK428 mRNA, complete cds. | PC3 |
| 180 | 1328236.5 | g474898 | 0 | Human cellular growth-regulating protein mRNA, complete cds. | PC3 |
| 181 | 995534.3 | g2290529 | 0 | Human WD repeat protein HAN11 mRNA, complete cds. | DU145 |
| 182 | 1098887.1 | g1698719 | 0 | Human zinc finger protein mRNA, complete cds. | DU145 |
| 183 | 268733.1 | g4753278 | 1.00E−132 | Homo sapiens PAC clone RP5-85011 from 7q31.2-q32, complete | PC3 & DU14 |
| 184 | 470023.2 | g177830 | 0 | Human alpha-1-antitrypsin gene (S variant), complete cds. | DU145 |
| 185 | 989966.8 | g3377596 | 0 | Human full length insert cDNA YO54H04. | DU145 |
| 186 | 233925.5 | g773643 | 0 | Human heterogeneous ribonucleoprotein A0 mRNA, complete cds. | PC3 |
| 187 | 405145.5 | g3252778 | 0 | Human mRNA for 3′,5′-cyclic GMP phosphodiesterase, complete cds. | DU145 |
| 188 | 261982.8 | g2306765 | 0 | Human zinc finger helicase (Znf-HX) mRNA, complete cds. | DU145 |
| 188 | 261982.8 | g2306765 | 0 | Human zinc finger helicase (Znf-HX) mRNA, complete cds. | PC3 |
| 189 | 246285.1 | g339432 | 0 | Human (clone CR-3) teratocarcinoma-derived growth factor 3 (TDGF3) mRNA, | DU145 |
| | | | | complete cds. | |
| 190 | 228511.1 | | | Incyte Unique | DU145 |
| 191 | 40290.1 | g1854440 | 4.9 | polyprotein [Turnip mosaic virus] | DU145 |
| 192 | 399465.3 | g1469194 | 0 | Human mRNA for KIAA0136 gene, partial cds. | DU145 |
| 193 | 898850.21 | g187382 | 0 | Human microtubule-associated protein 4 mRNA, complete cds. | PC3 & DU14 |
| 194 | 201356.1 | | | Incyte Unique | PC3 & DU14 |
| 195 | 223285.1 | g30413 | 1.00E−12 | Human dinucleotide repeat polymorphism at the D21S65 locus. | DU145 |
| 196 | 318000.4 | g5817188 | 0 | Human mRNA; cDNA DKFZp566L034 (from clone DKFZp566L034). | DU145 |
| 197 | 977887.1 | g1504017 | 0 | Human mRNA for KIAA0218 gene, complete cds. | PC3 |
| 198 | 1094199.1 | g2584879 | 0 | Human thyroid hormone receptor activator molecule (TRAM-1) mRNA, complete | PC3 |
| 199 | 1094984.12 | g2331249 | 7.00E−25 | Human Amplified in Breast Cancer (AIB1) mRNA, complete cds. | PC3 |
| 200 | 244785.3 | g4454679 | 0 | Human NADH-ubiquinone oxidoreductase subunit B14.5B homolog mRNA, | PC3 |
| | | | | complete cds. | |
| 201 | 242278.1c | | | | DU145 |
| 202 | 113621.5 | g5817115 | 0 | Human mRNA; cDNA DKFZp586J021 (from clone DKFZp586J021). | DU145 |
| 203 | 401532.2 | g1574997 | 0 | Human canalicular multispecific organic anion transporter (cMOAT), gene, | PC3 |
| | | | | complete cds. | |
| 204 | 006985.1c | | | | PC3 |
| 205 | 351209.14 | g556808 | 0 | Human genes for acid sphingomyelinase ASM. | PC3 & DU14 |
| 206 | 351209.16 | g402620 | 0 | Human mRNA for sphingomyelinase. | PC3 & DU14 |
| 207 | 475819.14 | g6523730 | 0 | Human DNA sequence from clone RP3-351K20 on chromosome | DU145 |
| 207 | 475819.14 | g7159799 | 1.00E−153 | dJ351K20.1.1 (novel C3HC4 type Zinc finger (RING finger) | DU145 |
| 208 | 241384.3 | g179400 | 0 | Human beta-D-galactosidase mRNA, complete cds. | PC3 |
| 209 | 3428.1 | g6006046 | 0 | Homo sapiens 2q35 BAC RPCI11-1064L18 (Roswell Park Cancer | DU145 |
| 210 | 244200.1 | g2292903 | 0 | Human GalNAc-T1 gene, 3′UTR. | DU145 |
| 211 | 412484.3 | | | Incyte Unique | PC3 |
| 212 | 412484.8 | | | Incyte Unique | PC3 |
| 213 | 402187.16 | g3811348 | 0 | Human cytosolic phospholipase A2 beta (cPLA2 beta) precursor RNA, complete | DU145 |
| | | | | sequence. | |
| 214 | 903091.31 | g1255988 | 0 | Human dystrobrevin-delta mRNA, complete cds. | DU145 |
| 215 | 903091.33c | g1255992 | 0 | Human dystrobrevin-gamma mRNA, complete cds. | PC3 |
| 216 | 903091.16 | g1256012 | 0 | Human dystrobrevin-beta mRNA, complete cds. | DU145 |
| 217 | 228447.29c | g338442 | 0 | Human general beta-spectrin (SPTBN1) mRNA, complete cds. | DU145 |
| 218 | 475473.1 | g437000 | 0 | Human microtubule-associated protein 1B (MAP1B) gene, complete cds. | DU145 |
| 219 | 354430.4 | g1665822 | 0 | Human mRNA for KIAA0280 gene, partial cds. | DU145 |
| 220 | 468221.12c | g2895078 | 0 | Human tumor protein D53 (TPD52L1) mRNA, partial cds. | DU145 |
| 221 | 468221.13 | g1469919 | 0 | Human D53 (hD53) mRNA, complete cds. | DU145 |
| 222 | 399187.1c | g576780 | 5.00E−20 | Human cyclin F mRNA, complete cds. | DU145 |
| 223 | 903338.12 | g181528 | 0 | Human defensin 1 protein mRNA, complete cds. | DU145 |
| 224 | 208529.1 | | | Incyte Unique | PC3 |
| 225 | 1040667.52 | g3882166 | 0 | Human mRNA for KIAA0723 protein, complete cds. | DU145 |
| 226 | 147665.1 | g4512267 | 5.00E−19 | Human DNA for Ig heavy-chain variable region, complete sequence, 1 of 5. | DU145 |
| 227 | 218524.4 | g559053 | 0 | Human interleukin 8 receptor beta (IL8RB) mRNA, complete cds. | DU145 |
| 228 | 346673.1 | g4218433 | 1.00E−47 | Human chromosome 22 CpG island DNA, genomic Mse1 fragment, clone | PC3 & DU14 |
| | | | | 22CGIB49B9, complete read. | |
| 229 | 58775.1 | | | Incyte Unique | DU145 |
| 230 | 197086.1 | g1881565 | 0 | Human cosmid g1572c101, complete sequence. | DU145 |
| 231 | 348143.7 | g1381163 | 0 | Human huntingtin interacting protein (HIP2) mRNA, complete cds. | DU145 |
| 232 | 239552.3c | g28976 | 0 | Human mRNA for azurocidin. | DU145 |
| 233 | 233003.6 | | | Incyte Unique | PC3 |
| 234 | 233003.2 | | | Incyte Unique | PC3 |
| 235 | 217860.1 | g4589637 | 0 | Human mRNA for KIAA0997 protein, complete cds. | DU145 |
| 236 | 216189.2 | g1580723 | 0 | Human mRNA for Tob, complete cds. | PC3 & DU14 |
| 237 | 343692.15c | g5733823 | 0 | Human ubiquilin mRNA, complete cds. | PC3 & DU14 |
| 238 | 382906.16 | g180142 | 0 | Human CD53 glycoprotein mRNA, complete cds. | DU145 |
| 239 | 482325.15 | g3043649 | 0 | Human mRNA for KIAA0563 protein, complete cds. | PC3 |
| 240 | 64851.1 | | | Incyte Unique | DU145 |
| 241 | 346016.5 | g4033734 | 0 | Human spliceosomal protein SAP 155 mRNA, complete cds. | DU145 |
| 242 | 346016.6 | g4033734 | 0 | Human spliceosomal protein SAP 155 mRNA, complete cds. | DU145 |
| 243 | 222222.1 | | | Incyte Unique | DU145 |
| 244 | 413835.5 | g2257985 | 0 | Human kruppel-related zinc finger protein hcKrox mRNA, complete cds. | PC3 |
| 245 | 978273.4 | g567834 | 2.00E−13 | Human (clone HG52) Z-crystallin/quinone reductase (CRYZ) gene sequence. | DU145 |
| 246 | 474673.1 | g687592 | 0 | Human p190-B (p190-B) mRNA, complete cds. | PC3 |
| 247 | 480286.1 | g510989 | 0 | Human genes for histones H2B.1 and H2A. | DU145 |
| 248 | 402640.1 | | | Incyte Unique | DU145 |
| 249 | 232218.1 | g473629 | 1.00E−64 | Human (clone 1NIB-138) normalized cDNA library sequence. | PC3 |
| 250 | 981919.1c | | | | PC3 |
| 251 | 229369.1 | g3169209 | 0 | Homo sapiens BAC clone CTA-300E22 from 7q21-q31.1, complete | PC3 |
| 252 | 253946.17 | g186353 | 0 | Human membrane glycoprotein gp130 mRNA, complete cds. | PC3 & DU14 |
| 253 | 13550.1 | | | Incyte Unique | DU145 |
| 254 | 997704.1 | g5926684 | 5.00E−13 | Human genomic DNA, chromosome 3p21.3, clone: 301 to 308, anti-oncogene | PC3 |
| | | | | region, section 5/5. | |
| 255 | 385608.45 | g2827202 | 0 | Human general transcription factor 2-I (GTF2I) mRNA, alternatively spliced | PC3 |
| | | | | product, complete cds. | |
| 256 | 385608.25c | g2687639 | 0 | Human general transcription factor 2-I pseudogene 1 (GTF2IP1) mRNA. | PC3 |
| 257 | 385608.3 | g2415381 | 0 | Human TFII-I protein (TFII-I) mRNA, complete cds. | PC3 |
| 258 | 78434.1 | g571295 | 0 | Human CRFB4 gene, partial cds. | DU145 |
| 259 | 243134.1 | g5678818 | 1.00E−08 | Human FRG1 (FRG1) gene, complete cds; 5S ribosomal RNA gene, complete | DU145 |
| | | | | sequence; TUB4q and TIG2 pseudogenes, complete sequence. | |
| 260 | 196959.4c | g1617112 | 0 | Human Na+-D-glucose cotransport regulator gene. | PC3 |
| 261 | 82168.5 | g6175872 | 0 | Human toll-like receptor 4 (TLR4) gene, TLR4A allele, complete cds. | PC3 |
| 262 | 4360.1 | g7023028 | 0 | Homo sapiens cDNA FLJ10785 fis, clone NT2RP4000457, weakly | DU145 |
| 263 | 412661.2 | g415818 | 0 | Human mki67a mRNA (long type) for antigen of monoclonal antibody Ki-67. | DU145 |
| 264 | 206310.2 | | | Incyte Unique | PC3 |
| 265 | 346599.14c | g178848 | 0 | Human apolipoprotein E mRNA, complete cds. | DU145 |
| 266 | 385608.47 | g2827202 | 0 | Human general transcription factor 2-I (GTF2I) mRNA, alternatively spliced | PC3 |
| | | | | product, complete cds. | |
| 267 | 385608.2 | g2827206 | 0 | Human general transcription factor 2-I (GTF2I) mRNA, alternatively spliced | PC3 |
| | | | | product, complete cds. | |
| 268 | 335202.1 | | | Incyte Unique | PC3 |
| 269 | 978402.3 | | | Incyte Unique | DU145 |
| 270 | 232146.1 | | | Incyte Unique | PC3 |
| 271 | 25757.1 | g5923890 | 0 | Human cyclophilin-related protein (NKTR) gene, complete cds. | DU145 |
| 272 | 206860.2 | | | Incyte Unique | PC3 |
| 273 | 239797.3 | g2909843 | 0 | Human prostate stem cell antigen (PSCA) mRNA, complete cds. | PC3 |
| 274 | 441328.12 | g4102966 | 0 | Human pre-mRNA splicing SR protein rA4 mRNA, partial cds. | PC3 |
| 275 | 113975.1 | g4827314 | 0 | Homo sapiens BAC clone RP11-365F8 from 7q31.1-q31.2, complete | PC3 |
| 276 | 427554.6 | | | Incyte Unique | DU145 |
| 277 | 360130.31c | g4099608 | 0 | Human cell division control-related protein 2b (hcdcrel2b) mRNA, complete cds. | DU145 |
| 278 | 82154.23 | | | Incyte Unique | PC3 |
| 279 | 82154.24 | | | Incyte Unique | PC3 |
| 280 | 1137293.16 | g3341991 | 1.00E−49 | Human histone macroH2A1.2 mRNA, complete cds. | PC3 |
| 281 | 994468.3 | | | Incyte Unique | PC3 |
| 282 | 994532.1 | | | Incyte Unique | PC3 |
| 283 | 90710.1 | g2342595 | 0 | Human DNA sequence from cosmid U221F2 on chromosome X. | PC3 & DU14 |
| 284 | 304359.1c | | | | PC3 & DU14 |
| 285 | 12402.1 | | | Incyte Unique | DU145 |
| 286 | 92743.1 | | | Incyte Unique | PC3 |
| 287 | 18513.1 | g6706246 | 0 | Human DNA sequence from clone RP3-393D12 on chromosome | PC3 |
| 288 | 477387.7 | g1036447 | 6.00E−72 | Human CpG island DNA genomic Msel fragment, clone 96f6, forward read | PC3 |
| | | | | cpg96f6.ftla. | |
| 289 | 350440.14c | g190096 | 0 | Human plasma membrane calcium-pumping ATPase (PMCA4) mRNA, complete | PC3 & DU14 |
| 290 | 350440.15 | g179162 | 0 | Human plasma membrane calcium ATPase (hPMCA4) mRNA, complete cds. | PC3 & DU14 |
| 291 | 992455.56c | g6048967 | 0 | Human clone H14 unknown mRNA. | DU145 |
| 292 | 997395.4 | g3483689 | 0 | Human full length insert cDNA clone ZD61F11. | PC3 |
| 293 | 17821.1 | | | Incyte Unique | PC3 |
| 294 | 201436.4c | g6093233 | 0 | Human mRNA; cDNA DKFZp566G1424 (from clone DKFZp566G1424). | PC3 |
| 295 | 201436.3 | g6093233 | 0 | Human mRNA; cDNA DKFZp566G1424 (from clone DKFZp566G1424). | PC3 |
| 296 | 198947.1 | | | Incyte Unique | DU145 |
| 297 | 1135407.1c | | | | DU145 |
| 298 | 200046.1 | g4156141 | 0 | Homo sapiens BAC clone RP11-436H22 from 2, complete sequence. | DU145 |
| 299 | 981489.1 | | | Incyte Unique | DU145 |
| 300 | 19362.1 | | | Incyte Unique | PC3 |
| 301 | 217116.1 | | | Incyte Unique | PC3 |
| 302 | 330530.1 | | | Incyte Unique | PC3 |
| 303 | 401213.1 | g598644 | 1.00E−57 | Human HepG2 partial cDNA, clone hmd2d12m5. | DU145 |
| 304 | 400280.3 | g4514553 | 0 | Human mRNA forRod1, complete cds. | PC3 |
| 305 | 17320.1 | g2736289 | 0 | Human hMed7 (MED7) mRNA, complete cds. | DU145 |
| 306 | 406182.1c | | | | PC3 |
| 307 | 244603.1 | g6065863 | 0 | Human DNA sequence from clone 1018E9 on chromosome | PC3 |
| 308 | 107405.1 | | | Incyte Unique | PC3 |
| 309 | 337024.4 | g299702 | 0 | 75 kda infertility-related sperm protein [Human, testis, mRNA Partial, 2427 nt]. | DU145 |
| 310 | 337024.3 | g299702 | 0 | 75 kda infertility-related sperm protein [Human, testis, mRNA Partial, 2427 nt]. | DU145 |
| 311 | 481848.1c | g2865218 | 0 | Human integrin binding protein Del-1 (Del1) mRNA, complete cds. | PC3 & DU14 |
| 312 | 208723.1 | | | Incyte Unique | DU145 |
| 313 | 64612.1 | | | Incyte Unique | PC3 |
| 314 | 978402.1 | | | Incyte Unique | DU145 |
| 315 | 453369.8 | g6642986 | 0 | Human aminopeptidase PILS (APPILS) mRNA, complete cds. | DU145 |
| 316 | 27240.1 | g5649181 | 1.00E−155 | Homo sapiens 3q26.2-27 BAC RPCI11-469J4 (Roswell Park Cancer | DU145 |
| 317 | 401290.1c | | | | DU145 |
| 318 | 481847.1 | g2865220 | 0 | Human integrin binding protein Del-1, Z20 splice variant, (Del1) mRNA, partial | PC3 & DU14 |
| 319 | 235636.1 | g2865218 | 1.00E−19 | Human integrin binding protein Del-1 (Del1) mRNA, complete cds. | PC3 & DU14 |
| 320 | 994057.7 | g37137 | 3.00E−79 | Human mRNA for thrombospondin. | DU145 |
| 321 | 405559.1 | g897824 | 1.70E−17 | AHNAK gene product | DU145 |
| 322 | 16760.1 | | | Incyte Unique | DU145 |
| 323 | 198522.1 | | | Incyte Unique | PC3 |
| 324 | 19366.1 | | | Incyte Unique | DU145 |
| 325 | 002679.7c | | | | PC3 |
| 326 | 208276.1 | | | Incyte Unique | DU145 |
| 327 | 21552.1 | g3688074 | 0 | Homo sapiens chromosome 5, BAC clone 34j15 (LBNL H169), | PC3 |
| 328 | 198087.1 | g4160665 | 0 | Human cDNA for NG,NG-dimethylarginine dimethylaminohydrolase, complete | PC3 |
| 329 | 903269.4 | g3661609 | 0 | Human splicing factor Prp8 mRNA, complete cds. | PC3 |
| 330 | 351166.1 | | | Incyte Unique | DU145 |
| 331 | 344713.3 | g6472600 | 0.29 | unconventional myosin heavy chain [Chara corallina] | DU145 |
| 332 | 167920.1 | g2636170 | 1.7 | similar to antibiotic resistance protein [Bacillus subtilis] | DU145 |
| 333 | 433569.1 | g791003 | 0 | Human ARSE gene, complete cds. | PC3 |
| 334 | 406365.1 | | | Incyte Unique | PC3 |
| 335 | 348121.9 | g1136417 | 0 | Human mRNA for KIAA0179 gene, partial cds. | PC3 |
| 336 | 363585.1c | | | | PC3 |
| 337 | 408116.1 | | | Incyte Unique | PC3 |
| 338 | 339737.1c | | | | DU145 |
| 339 | 978146.1 | | | Incyte Unique | PC3 |
| 340 | 186012.1 | | | Incyte Unique | DU145 |
| 341 | 405041.1 | g3929221 | 2.40E−102 | TRF1-interacting ankyrin-related ADP-ribose polymerase | DU145 |
| 342 | 474266.2 | g3929218 | 7.00E−28 | Human TRF1-interacting ankyrin-related ADP-ribose polymerase mRNA, | DU145 |
| | | | | complete cds. | |
| 343 | 401530.2 | g6731234 | 4.00E−75 | Human myoferlin (MYOF) mRNA, complete cds. | DU145 |
| 344 | 234729.11 | g6330496 | 0 | Human mRNA for KIAA1207 protein, partial cds. | DU145 |
| 345 | 195199.1 | g6329818 | 0 | Human mRNA for KIAA1131 protein, partial cds. | DU145 |
| 346 | 368731.1 | | | Incyte Unique | PC3 |
| 347 | 372313.6 | g885977 | 0 | Human organic anion transporting polypeptide (OATP) mRNA, complete cds. | PC3 |
| 348 | 256871.2 | g3861482 | 2.00E−86 | Human chromosome 3, olfactory receptor pseudogene cluster 1, complete | DU145 |
| | | | | sequence, and myosin light chain kinase (MLCK) pseudogene, partial sequence. | |
| 349 | 256871.16c | g896064 | 0 | Human protein immuno-reactive with anti-PTH polyclonal antibodies mRNA, | DU145 |
| | | | | partial cds. | |

FGF treated, upregulated

| | | | | | |
|---|---|---|---|---|---|
| 350 | 22485.15 | g34349 | 0 | Human LFA-3 mRNA for glycosylated surface protein. | DU145 |
| 351 | 22485.1 | g34346 | 0 | Human mRNA for lymphocyte function associated antigen-3 (LFA-3). | DU145 |
| 352 | 26410.1 | g2381480 | 0 | Human mRNA for epiregulin, complete cds. | DU145 |
| 353 | 215720.1 | | | Incyte Unique | DU145 |
| 354 | 472165.22 | g662993 | 0 | Human mRNA encoding GPI-anchored protein p137. | DU145 |
| 355 | 206580.1 | | | Incyte Unique | DU145 |
| 356 | 344775.3 | g3822553 | 0 | nuclear calmodulin-binding protein [Gallus gallus] | DU145 |
| 357 | 334668.1 | | | Incyte Unique | DU145 |
| 358 | 416874.3 | g340159 | 0 | Human pro-urokinase mRNA, complete cds. | DU145 |
| 359 | 427964.2 | g31462 | 0 | Human fra-1 mRNA. | DU145 |
| 360 | 234537.3c | g23896 | 0 | Human placental cDNA coding for 5′nucleotidase (EC 3.1.3.5). | DU145 |
| 361 | 31760.1 | | | Incyte Unique | DU145 |
| 362 | 336953.7 | g1813423 | 0 | Human mRNA for HCS, complete cds. | DU145 |
| 363 | 444771.2 | g31107 | 0 | Human mRNA for elongation factor 2. | DU145 |
| 364 | 449173.16 | g189499 | 0 | Human p62 mRNA, complete cds. | DU145 |
| 365 | 1523.1 | | | Incyte Unique | DU145 |
| 366 | 239097.1 | | | Incyte Unique | DU145 |
| 367 | 252747.27 | g6330689 | 0 | Human mRNA for KIAA1228 protein, partial cds. | DU145 |
| 368 | 482490.11 | g6650213 | 8.00E−69 | Human RAN binding protein 16 mRNA, complete cds. | DU145 |
| 369 | 1000084.27 | g3719220 | 0 | Human vascular endothelial growth factor mRNA, complete cds. | DU145 |

TGFβ treated, downregulated

| | | | | | |
|---|---|---|---|---|---|
| 263 | 412661.2 | g415818 | 0 | Human mki67a mRNA (long type) for antigen of monoclonal antibody Ki-67. | DU145 |
| 316 | 27240.1 | g5649181 | 1.00E−155 | Homo sapiens 3q26.2-27 BAC RPCI11-469J4 (Roswell Park Cancer | DU145 |
| 370 | 205607.5 | g987947 | 0 | Human mRNA for phosphatidylinositol 3-kinase. | DU145 |
| 371 | 1089426.1 | g886947 | 0.54 | orf3 [Saccharomyces cerevisiae] | DU145 |
| 372 | 335186.2 | g3929751 | 2.00E−11 | Human SYBL1 gene. | DU145 |
| 373 | 357276.8 | g2232030 | 0 | Human inositol polyphosphate 4-phosphatase type I-beta mRNA, complete cds. | DU145 |
| 374 | 474724.5 | g507212 | 0 | Human serine kinase mRNA, complete cds. | DU145 |
| 375 | 230889.3 | g5817126 | 0 | Human mRNA; cDNA DKFZp586P1622 (from clone DKFZp586P1622). | DU145 |
| 376 | 903909.1 | | | Incyte Unique | DU145 |
| 377 | 229298.1 | g4500162 | 0 | Human mRNA; cDNA DKFZp586D0918 (from clone DKFZp586D0918). | DU145 |
| 378 | 229298.2 | g4500162 | 0 | Human mRNA; cDNA DKFZp586D0918 (from clone DKFZp586D0918). | DU145 |
| 379 | 110678.1 | g4371263 | 0 | Homo sapiens chromosome 16 clone 66H6, complete sequence. | DU145 |
| 380 | 239093.1 | | | Incyte Unique | DU145 |
| 381 | 237963.11c | g3873560 | 0 | Human mRNA for C17orf1 protein. | DU145 |
| 382 | 237963.8 | g3873560 | 0 | Human mRNA for C17orf1 protein. | DU145 |
| 383 | 400135.1 | | | Incyte Unique | DU145 |
| 384 | 344398.2 | g4500170 | 0 | Human mRNA; cDNA DKFZp586K1318 (from clone DKFZp586K1318). | DU145 |
| 385 | 1086647.1 | | | Incyte Unique | DU145 |
| 386 | 1682.1 | | | Incyte Unique | DU145 |
| 387 | 010190.1c | | | | DU145 |
| 388 | 205311.1 | g5931461 | e−149 | Homo sapiens clone NH0309N08, complete sequence. | DU145 |
| 389 | 22429.7 | g457372 | e−129 | dihydroxypolyprenylbenzoate methyltransferase [Rattus | DU145 |
| 390 | 59379.1 | | | Incyte Unique | DU145 |
| 391 | 236253.1 | g6808164 | 4.00E−83 | Human mRNA; cDNA DKFZp761A052 (from clone DKFZp761A052). | DU145 |
| 392 | 331033.1 | g286012 | 0 | Human mRNA for KIAA0008 gene, complete cds. | DU145 |
| 393 | 345533.8c | g2385368 | 0 | Human mRNA for Rer1 protein. | DU145 |
| 394 | 238342.1 | g3483461 | 0 | Human full length insert cDNA clone ZA70C11. | DU145 |
| 395 | 28936.1 | g3282164 | 0 | Homo sapiens chromosome 5, BAC clone 282B7 (LBNL H192), | DU145 |
| 396 | 347865.5 | g6807697 | 2.00E−40 | Human mRNA; cDNA DKFZp434A1014 (from clone DKFZp434A1014); partial | DU145 |
| 397 | 347865.4 | g6807697 | 1.00E−89 | Human mRNA; cDNA DKFZp434A1014 (from clone DKFZp434A1014); partial | DU145 |
| 398 | 40057.2 | g285946 | 0 | Human mRNA for KIAA0105 gene, complete cds. | DU145 |
| 399 | 997301.6 | | | Incyte Unique | DU145 |
| 400 | 12235.1 | | | Incyte Unique | DU145 |
| 401 | 245496.7 | g7298512 | 4.00E−54 | CG10641 gene product [Drosophila melanogaster] | DU145 |
| 402 | 983262.3c | | | | DU145 |
| 403 | 998084.1 | g3483880 | 0 | Human full length insert cDNA clone ZE07G05. | DU145 |
| 404 | 238071.2 | g5926700 | 0 | Human genomic DNA, chromosome 6p21.3, HLA Class I region, section 12/20. | DU145 |
| 405 | 103930.1 | | | Incyte Unique | DU145 |
| 406 | 254068.1 | g3095056 | 1.00E−08 | Human platelet-activating factor acetylhydrolase gene, promoter region and exon | DU145 |
| 407 | 221812.1 | g7019862 | 0 | Homo sapiens cDNA FLJ20033 fis, clone COL00106. | DU145 |
| 408 | 240129.1 | | | Incyte Unique | DU145 |
| 409 | 230297.1c | | | | DU145 |
| 410 | 347796.7 | g710405 | 2.8 | 35 kDa protein [Bartonella henselae] | DU145 |
| 411 | 411474.17 | g3043687 | 0 | Human mRNA for KIAA0582 protein, partial cds. | DU145 |
| 412 | 027434.1c | | | | DU145 |
| 413 | 979488.1c | | | | DU145 |
| 414 | 213988.1 | g7263867 | 1.00E−134 | Human chromosome 14 DNA sequence *** IN PROGRESS *** BAC | DU145 |

TGFβ treated, upregulated

| | | | | | |
|---|---|---|---|---|---|
| 321 | 405559.1 | g897824 | 1.70E−17 | AHNAK gene product | DU145 |
| 322 | 16760.1 | | | Incyte Unique | DU145 |
| 415 | 263336.62 | g187530 | 0 | Human metallothionein-II pseudogene (mt-IIps). | DU145 |
| 416 | 153860.6 | g4508112 | 0 | Homo sapiens clone RG161A02, complete sequence. | DU145 |
| 417 | 154178.1 | | | Incyte Unique | DU145 |
| 418 | 337888.3 | | | Incyte Unique | DU145 |
| 419 | 240009.2c | | | | DU145 |
| 420 | 160011.1 | | | Incyte Unique | DU145 |
| 421 | 332919.4 | g793840 | 0 | Human mRNA for cytokine inducible nuclear protein. | DU145 |
| 422 | 6233.1 | | | Incyte Unique | DU145 |
| 423 | 245532.17c | g495286 | 0 | Human melanoma differentiation associated (mda-6) mRNA, complete cds. | DU145 |
| 424 | 205486.1 | | | Incyte Unique | DU145 |
| 425 | 987927.13 | g458227 | 0 | Human extracellular protein (S1-5) mRNA, complete cds. | DU145 |
| 426 | 25423.3 | | | Incyte Unique | DU145 |
| 427 | 1091415.2 | g5231136 | 0 | Human angiopoietin-related protein mRNA, complete cds. | DU145 |
| 428 | 1091415.16c | g5231136 | 0 | Human angiopoietin-related protein mRNA, complete cds. | DU145 |
| 429 | 230058.2 | g4154282 | 0 | Human xenotropic and polytropic murine leukemia virus receptor (X3) mRNA, | DU145 |
| | | | | complete cds. | |
| 430 | 444648.9 | g187542 | 0 | Human metallothionein (MT)I-F gene, complete cds. | DU145 |
| 431 | 464689.22 | g4808600 | 0 | Human stearoyl-CoA desaturase (SCD) mRNA, complete cds. | DU145 |
| 432 | 464689.15 | g4808600 | 0 | Human stearoyl-CoA desaturase (SCD) mRNA, complete cds. | DU145 |
| 433 | 334809.3c | g36510 | 0 | Human sno oncogene mRNA for snoN protein, ski-related. | DU145 |
| 434 | 351241.1 | g2935483 | 4.00E−56 | Human minisatellite cebl repeat region. | DU145 |
| 435 | 482336.11 | g34067 | 0 | Human mRNA fragment for mesothelial type II keratin K7. | DU145 |
| 436 | 482336.31 | g34067 | 0 | Human mRNA fragment for mesothelial type II keratin K7. | DU145 |

TGFα treated, downregulated

| | | | | | |
|---|---|---|---|---|---|
| 43 | 60671.7 | g3043675 | 0 | Human mRNA for KIAA0576 protein, partial cds. | PC3 |
| 437 | 197538.2 | g942584 | 0 | Human RAR-responsive (TIG1) mRNA, complete cds. | PC3 |
| 438 | 197538.8 | g6066619 | 7.00E−06 | latexin [Rattus norvegicus] | PC3 |
| 439 | 232048.14 | g37638 | 0 | Human mRNA for vascular anticoagulant-beta (VAC-beta). | PC3 |
| 440 | 1092381.1 | g6634028 | 0 | Human mRNA for KIAA0399 protein, partial cds. | PC3 |
| 441 | 903804.1 | g5815499 | 0 | Homo sapiens 12p12-27.2-31.7 BAC RPCI11-392P7 (Roswell Park | PC3 |
| 442 | 210945.6 | g532500 | 0 | Human mRNA for FK506-binding protein 12 kDa (hFKBP-12) homologue, | PC3 |
| | | | | complete cds. | |
| 443 | 210945.3c | g532500 | 0 | Human mRNA for FK506-binding protein 12 kDa (hFKBP-12) homologue, | PC3 |
| | | | | complete cds. | |
| 444 | 403448.2 | g3037018 | 3.8 | NADH dehydrogenase subunit 5 [Bodo saltans] | PC3 |
| 445 | 238263.2 | | | Incyte Unique | PC3 |
| 446 | 146382.22 | g2463541 | 0 | Human mRNA for inositol 1,4,5-trisphosphate 3-kinase isoenzyme, partial cds. | PC3 |
| 447 | 146382.25 | g2463541 | 0 | Human mRNA for inositol 1,4,5-trisphosphate 3-kinase isoenzyme, partial cds. | PC3 |
| 448 | 345705.7 | g3047307 | 0 | Human sarcosin mRNA, complete cds. | PC3 |
| 449 | 198212.1 | g4416529 | 0 | Human skeletal muscle LIM-protein FHL3 mRNA, complete cds. | PC3 |
| 450 | 198212.6 | g4416529 | 0 | Human skeletal muscle LIM-protein FHL3 mRNA, complete cds. | PC3 |
| 451 | 900031.8 | g2094872 | 0 | Human DAP-kinase mRNA. | PC3 |
| 452 | 900031.4 | g2094872 | 0 | Human DAP-kinase mRNA. | PC3 |
| 453 | 475365.4 | | | Incyte Unique | PC3 |
| 454 | 222810.1 | g182662 | 0 | Human N-formylpeptide receptor (fMLP-R98) mRNA, complete cds. | PC3 |
| 455 | 1084493.6 | g1150990 | 0 | Human receptor tyrosine kinase Flt4 (short form) mRNA, complete cds. | PC3 |
| 456 | 979005.2 | g4827155 | 3 | bcsCI [Acetobacter xylinus] | PC3 |
| 457 | 346686.23 | g183046 | 0 | Human granulocyte colony-stimulating factor receptor (G-CSFR-1) mRNA, | PC3 |
| | | | | complete cds. | |
| 458 | 234568.25 | g1469204 | 0 | Human mRNA for KIAA0141 gene, complete cds. | PC3 |
| 459 | 234568.17 | g1469204 | 0 | Human mRNA for KIAA0141 gene, complete cds. | PC3 |
| 460 | 978276.1c | | | | PC3 |
| 461 | 26968.1 | g2253289 | 3.00E−09 | Human endothelin A receptor gene, 5' flanking region and exon 1. | PC3 |
| 462 | 243103.24 | g562105 | 0 | Human (dlk) mRNA, complete cds. | PC3 |
| 463 | 469883.1 | g1663566 | 0 | Human semaphorin (CD100) mRNA, complete cds. | PC3 |
| 464 | 903565.14c | g2330552 | 0 | Human mRNA for PACE4A-II, complete cds. | PC3 |
| 465 | 227932.2 | g2264346 | 1.00E−141 | GOK [Homo sapiens] | PC3 |
| 466 | 903691.6 | g439295 | 0 | Human garp gene mRNA, complete cds. | PC3 |
| 467 | 012995.19c | g6682360 | 0 | Human talin mRNA, complete cds. | PC3 |
| 468 | 407938.5 | g2765321 | 0 | Human mRNA for RB18A protein. | PC3 |
| 469 | 233778.1 | g487808 | 0 | Human cell surface protein (NKG7) mRNA, complete cds. | PC3 |
| 470 | 474630.19 | g33910 | 0 | Human mRNA for integrin beta(4)subunit. | PC3 |
| 471 | 217319.7 | g1033874 | 7.00E−11 | Human CpG island DNA genomic Msel fragment, clone 53c10, reverse read | PC3 |
| | | | | cpg53c10.rtlb. | |
| 472 | 1092445.1 | g5915897 | 0 | Human zinc finger protein 42 (ZNF42) mRNA, alternate transcript, complete cds. | PC3 |
| 473 | 199433.3 | g4323621 | 0 | Human intracellular chloride channel CLIC3 (CLIC3) mRNA, complete cds. | PC3 |
| 474 | 228860.6 | g2865608 | 0 | Human homogentisate 1,2-dioxygenase (AKU) mRNA, complete cds. | PC3 |
| 475 | 228860.4c | g2130646 | 0 | Human homogentisate 1,2-dioxygenase gene, complete cds. | PC3 |
| 476 | 410688.3 | g1405359 | 9.00E−89 | Human fetal brain (239FB) mRNA, from the WAGR region, complete cds. | PC3 |
| 477 | 347005.5 | g35787 | 0 | Human HPTP beta mRNA for protein tyrosine phosphatase beta. | PC3 |
| 478 | 334621.13 | g3599961 | 5.00E−90 | Human h-bcs1 (BCS1) mRNA, nuclear gene encoding mitochondrial protein, | PC3 |
| | | | | complete cds. | |
| 479 | 28180.1 | | | Incyte Unique | PC3 |
| 480 | 252570.6c | g3820529 | 7.2 | protein kinase homolog; AbiBL11 [Bacillus licheniformis] | PC3 |
| 481 | 466402.116 | g435059 | 0 | Human Golli-mbp gene, complete cds. | PC3 |
| 482 | 1096160.26c | g561542 | 0 | Human protein kinase (zpk) mRNA, complete cds. | PC3 |
| 483 | 899612.2 | g4884371 | 0 | Human mRNA; cDNA DKFZp586A0522 (from clone DKFZp586A0522); partial | PC3 |
| 484 | 254107.1 | g220126 | 0 | Human gene for thrombomodulin precursor, complete cds. | PC3 |
| 485 | 208328.1 | g2546963 | 0 | Human mRNA for diubiquitin. | PC3 |
| 486 | 130157.2 | g5870324 | 1.00E−61 | supported by human EST AA331940 (NID: g1984182), mouse EST AA387866 | PC3 |
| | | | | (NID: 2040812), and Genscan [Homo sapiens] | |
| 487 | 985824.2c | | | | PC3 |
| 488 | 233089.1 | g7242976 | 0 | Homo sapiens mRNA for KIAA1311 protein, partial cds. | PC3 |
| 489 | 336256.1 | g963053 | 0 | Human mRNA for membrane-type matrix metalloprotease 1. | PC3 |
| 490 | 979616.2 | g563140 | 2.4 | cytochrome c oxidase subunit 1 [Trypanoplasma borreli] | PC3 |
| 491 | 351432.3 | g1809029 | 0 | Human AE2 anion exchanger (SLC4A2) mRNA, complete cds. | PC3 |
| 492 | 83495.1 | | | Incyte Unique | PC3 |
| 493 | 332595.1 | g2979421 | 0 | Human mRNA for PCDH7 (BH-Pcdh)c, complete cds. | PC3 |
| 494 | 481251.3 | g5917667 | 0 | Human cysteine-rich hydrophobic 2 CHIC2 mRNA, complete cds. | PC3 |
| 495 | 25612.1 | | | Incyte Unique | PC3 |
| 496 | 96700.1 | | | Incyte Unique | PC3 |
| 497 | 230048.1 | | | Incyte Unique | PC3 |
| 498 | 073621.3c | g6330384 | 0 | Human mRNA for KIAA1197 protein, partial cds. | PC3 |
| 499 | 344582.19 | g4106877 | 0 | Human decoy receptor 3 (DcR3) mRNA, complete cds. | PC3 |
| 500 | 997526.4 | g5102629 | 0 | Novel Human gene mapping to chomosome 22. | PC3 |
| 501 | 997526.1 | g3483012 | 0 | Human mRNA for HMGBCG protein. | PC3 |

TABLE 2

| SEQ | TEMPLATE | | | | | |
|---|---|---|---|---|---|---|
| ID NO | ID | START | STOP | FRAME | Pfam Description | E-Value |
| 3 | 977955.7 | 984 | 1133 | forward 3 | Phorbol esters/diacylglycerol binding domain (C1 domain) | 2.40E−21 |
| 3 | 977955.7 | 1440 | 1796 | forward 3 | PH domain | 7.10E−12 |
| 3 | 977955.7 | 1920 | 2690 | forward 3 | Eukaryotic protein kinase domain | 5.70E−76 |
| 4 | 350754.2 | 857 | 1123 | forward 2 | Transglutaminase-like superfamily | 1.40E−46 |
| 4 | 350754.2 | 1457 | 2119 | forward 2 | Transglutaminase family | 3.20E−50 |
| 4 | 350754.2 | 56 | 415 | forward 2 | Transglutaminase family | 2.20E−63 |
| 5 | 235191.4 | 180 | 617 | forward 3 | Ubiquitin-conjugating enzyme | 3.30E−57 |
| 7 | 1099593.2 | 325 | 447 | forward 1 | Armadillo/beta-catenin-like repeats | 2.20E−07 |
| 11 | 347915.14 | 1585 | 1911 | forward 1 | PH domain | 2.00E−14 |
| 11 | 347915.14 | 2191 | 2436 | forward 1 | Src homology domain 2 | 1.90E−21 |
| 12 | 997395.1 | 2027 | 2143 | forward 2 | WD domain, G-beta repeat | 2.10E−05 |
| 16 | 404155.2 | 2133 | 2417 | forward 3 | Cyclic nucleotide-binding domain | 6.20E−30 |
| 16 | 404155.2 | 2587 | 3207 | forward 1 | Eukaryotic protein kinase domain | 3.30E−71 |
| 16 | 404155.2 | 2505 | 2669 | forward 3 | Eukaryotic protein kinase domain | 2.20E−06 |
| 16 | 404155.2 | 3208 | 3306 | forward 1 | Protein kinase C terminal domain | 1.30E−04 |
| 19 | 345860.21 | 459 | 1109 | forward 3 | Papain family cysteine protease | 2.80E−133 |
| 20 | 199101.1 | 612 | 1172 | forward 3 | Glycosyl transferases | 9.60E−18 |
| 20 | 199101.1 | 1554 | 1940 | forward 3 | Similarity to lectin domain of ricin beta-chain, 3 copies. | 3.00E−09 |
| 22 | 475146.3 | 197 | 1567 | forward 2 | Hydroxymethylglutaryl-coenzyme A synthase | 0.00E+00 |
| 24 | 255115.2 | 91 | 1605 | forward 1 | UDP-glucoronosyl and UDP-glucosyl transferases | 0.00E+00 |
| 25 | 255115.4 | 121 | 1023 | forward 1 | UDP-glucoronosyl and UDP-glucosyl transferases | 1.70E−177 |
| 25 | 255115.4 | 977 | 1573 | forward 2 | UDP-glucoronosyl and UDP-glucosyl transferases | 2.60E−158 |
| 29 | 978047.1 | 613 | 1287 | forward 1 | alpha/beta hydrolase fold | 1.20E−04 |
| 37 | 399067.1 | 1426 | 1611 | forward 1 | Immunoglobulin domain | 1.50E−08 |
| 37 | 399067.1 | 538 | 609 | forward 1 | Leucine Rich Repeat | 1.40E−04 |
| 37 | 399067.1 | 1219 | 1377 | forward 1 | Leucine rich repeat C-terminal domain | 9.90E−13 |
| 37 | 399067.1 | 199 | 321 | forward 1 | Leucine rich repeat N-terminal domain | 6.70E−04 |
| 41 | 255824.52 | 642 | 1244 | forward 3 | Fructose-bisphosphate aldolase class-I | 5.60E−149 |
| 41 | 255824.52 | 1237 | 1644 | forward 1 | Fructose-bisphosphate aldolase class-I | 7.30E−96 |
| 44 | 376085.9 | 1596 | 1664 | forward 3 | Zinc finger, C2H2 type | 2.10E−05 |
| 45 | 404467.1 | 6 | 764 | forward 3 | 7 transmembrane receptor (Secretin family) | 6.30E−09 |
| 49 | 391406.5 | 2183 | 2299 | forward 2 | WD domain, G-beta repeat | 1.60E−13 |
| 49 | 391406.5 | 1128 | 1244 | forward 3 | WD domain, G-beta repeat | 1.60E−13 |
| 50 | 391406.24 | 349 | 465 | forward 1 | WD domain, G-beta repeat | 1.60E−13 |
| 50 | 391406.24 | 87 | 206 | forward 3 | WD domain, G-beta repeat | 2.10E−07 |
| 51 | 474588.21 | 2263 | 2460 | forward 1 | RNA recognition motif. (a.k.a. RRM, RBD, or RNP domain) | 9.20E−21 |
| 51 | 474588.21 | 1863 | 2075 | forward 3 | RNA recognition motif. (a.k.a. RRM, RBD, or RNP domain) | 6.90E−09 |
| 53 | 246862.9 | 1391 | 1489 | forward 2 | ATP synthase Alpha chain, C terminal | 1.20E−22 |
| 53 | 246862.9 | 1315 | 1401 | forward 1 | ATP synthase Alpha chain, C terminal | 4.10E−20 |
| 53 | 246862.9 | 347 | 1315 | forward 2 | ATP synthase alpha/beta family | 1.10E−87 |
| 53 | 246862.9 | 307 | 1071 | forward 1 | ATP synthase alpha/beta family | 9.60E−10 |
| 54 | 246862.17 | 1428 | 1832 | forward 3 | ATP synthase Alpha chain, C terminal | 4.30E−106 |
| 54 | 246862.17 | 303 | 1427 | forward 3 | ATP synthase alpha/beta family | 1.60E−187 |
| 56 | 236480.3 | 2154 | 2264 | forward 3 | WD domain, G-beta repeat | 7.30E−08 |
| 57 | 314831.5 | 841 | 2037 | forward 1 | Elongation factor Tu family | 6.10E−142 |
| 58 | 391940.1 | 1300 | 2004 | forward 1 | alpha/beta hydrolase fold | 1.70E−31 |
| 59 | 454958.13 | 266 | 799 | forward 2 | Tissue inhibitor of metalloproteinases | 1.80E−141 |
| 61 | 238203.11 | 89 | 3544 | forward 2 | Vinculin family | 9.30E−281 |
| 61 | 238203.11 | 828 | 3182 | forward 3 | Vinculin family | 4.50E−110 |
| 62 | 994057.21 | 277 | 1155 | forward 1 | Ser/Thr protein phosphatase | 6.10E−202 |
| 63 | 346730.5 | 71 | 1300 | forward 2 | Monocarboxylate transporter | 1.10E−48 |
| 66 | 482336.2 | 768 | 1706 | forward 3 | Intermediate filament proteins | 5.50E−167 |
| 67 | 482336.14 | 654 | 1592 | forward 3 | Intermediate filament proteins | 1.30E−167 |
| 69 | 300294.3 | 589 | 1392 | forward 1 | SRP54-type protein | 4.00E−141 |
| 71 | 475028.7 | 203 | 721 | forward 2 | ADP-ribosylation factor family | 8.10E−06 |
| 71 | 475028.7 | 233 | 853 | forward 2 | Ras family | 2.90E−92 |
| 72 | 481223.3 | 430 | 636 | forward 1 | HMG (high mobility group) box | 3.80E−31 |
| 74 | 234340.7 | 1164 | 1412 | forward 3 | Ets-domain | 1.80E−53 |
| 77 | 317586.1 | 471 | 1253 | forward 3 | N-acetyltransferase | 1.40E−208 |
| 86 | 1063057.1 | 153 | 221 | forward 3 | Zinc finger, C2H2 type | 1.60E−06 |
| 87 | 199905.1 | 48 | 200 | forward 3 | Laminin EGF-like (Domains III and V) | 8.90E−13 |
| 90 | 330878.6 | 178 | 2286 | forward 1 | Cullin family | 2.40E−222 |
| 91 | 331793.11 | 175 | 819 | forward 1 | emp24/gp25L/p24 family | 4.50E−55 |
| 97 | 977667.1 | 273 | 1013 | forward 3 | 7 transmembrane receptor (rhodopsin family) | 4.40E−77 |
| 99 | 1136709.5 | 284 | 601 | forward 2 | Vertebrate galactoside-binding lectins | 1.60E−58 |
| 100 | 1136709.6 | 137 | 454 | forward 2 | Vertebrate galactoside-binding lectins | 1.80E−57 |
| 101 | 382293.16 | 756 | 1094 | forward 3 | CUB domain | 4.00E−42 |
| 101 | 382293.16 | 636 | 746 | forward 3 | EGF-like domain | 1.40E−06 |
| 101 | 382293.16 | 1308 | 1496 | forward 3 | Sushi domain (SCR repeat) | 1.20E−12 |
| 101 | 382293.16 | 1545 | 2258 | forward 3 | Trypsin | 3.30E−68 |
| 102 | 382293.15 | 988 | 1326 | forward 1 | CUB domain | 4.00E−42 |
| 102 | 382293.15 | 868 | 978 | forward 1 | EGF-like domain | 1.40E−06 |
| 102 | 382293.15 | 1567 | 1755 | forward 1 | Sushi domain (SCR repeat) | 1.20E−12 |
| 102 | 382293.15 | 1804 | 2517 | forward 1 | Trypsin | 3.30E−68 |
| 104 | 235943.36 | 184 | 531 | forward 1 | SEA domain | 2.40E−29 |
| 105 | 401434.1 | 410 | 604 | forward 2 | DnaJ domain | 9.70E−35 |
| 106 | 234107.2 | 1162 | 1311 | forward 1 | Immunoglobulin domain | 4.60E−08 |
| 106 | 234107.2 | 177 | 422 | forward 3 | Immunoglobulin domain | 2.50E−05 |
| 109 | 441298.14 | 26 | 175 | forward 2 | PHD-finger | 1.40E−11 |
| 113 | 230936.6 | 184 | 996 | forward 1 | Ribosomal RNA adenine dimethylases | 8.10E−32 |
| 116 | 405158.1 | 23 | 121 | forward 2 | Ank repeat | 1.20E−04 |
| 116 | 405158.1 | 756 | 986 | forward 3 | IBR domain | 9.00E−15 |
| 118 | 480187.81 | 305 | 538 | forward 2 | Immunoglobulin domain | 2.40E−08 |
| 119 | 62042.4 | 247 | 492 | forward 1 | Fibronectin type III domain | 1.40E−11 |
| 120 | 1087696.7 | 179 | 769 | forward 2 | short chain dehydrogenase | 9.20E−45 |
| 121 | 259805.28 | 328 | 1083 | forward 1 | Eukaryotic protein kinase domain | 5.20E−100 |
| 121 | 259805.28 | 1141 | 1257 | forward 1 | UBA domain | 6.00E−06 |
| 123 | 986565.15 | 241 | 1533 | forward 1 | Reduced folate carrier | 9.10E−143 |
| 124 | 978378.3 | 361 | 999 | forward 1 | TBC domain | 1.90E−37 |
| 127 | 238635.1 | 123 | 296 | forward 3 | Immunoglobulin domain | 2.60E−09 |
| 128 | 1080496.1 | 1256 | 1324 | forward 2 | Zinc finger, C2H2 type | 9.80E−04 |
| 129 | 351204.2 | 190 | 777 | forward 1 | Trypsin | 5.70E−76 |
| 133 | 474868.2 | 209 | 403 | forward 2 | DnaJ domain | 1.20E−36 |
| 133 | 474868.2 | 866 | 1234 | forward 2 | DnaJ C terminal region | 2.50E−18 |
| 140 | 411296.2 | 1186 | 1923 | forward 1 | Eukaryotic protein kinase domain | 9.90E−73 |
| 140 | 411296.2 | 832 | 1080 | forward 1 | Src homology domain 2 | 2.00E−50 |
| 140 | 411296.2 | 643 | 810 | forward 1 | SH3 domain | 2.70E−25 |
| 150 | 346511.4 | 1129 | 1449 | forward 1 | Nucleotidyltransferase domain | 8.90E−17 |
| 151 | 346511.6 | 1124 | 1444 | forward 2 | Nucleotidyltransferase domain | 8.90E−17 |
| 153 | 234056.5 | 162 | 899 | forward 3 | Transmembrane 4 family | 1.60E−47 |
| 156 | 988653.1 | 1295 | 1369 | forward 2 | Zinc finger, C2H2 type | 1.00E−06 |
| 157 | 470468.26 | 726 | 1472 | forward 3 | Phosphorylase family | 1.20E−04 |
| 161 | 1094412.1 | 298 | 2118 | forward 1 | Hsp70 protein | 0.00E+00 |
| 162 | 1094107.1 | 293 | 2104 | forward 2 | Hsp70 protein | 4.80E−204 |
| 162 | 1094107.1 | 813 | 2066 | forward 3 | Hsp70 protein | 1.30E−31 |
| 164 | 12178.2 | 15 | 248 | forward 3 | E1-E2 ATPase | 1.20E−09 |
| 165 | 100653.3 | 313 | 486 | forward 1 | Helix-loop-helix DNA-binding domain | 3.20E−13 |
| 171 | 331065.2 | 244 | 510 | forward 1 | Caspase recruitment domain | 3.70E−32 |
| 171 | 331065.2 | 1226 | 1507 | forward 2 | ICE-like protease (caspase) p10 domain | 1.60E−50 |
| 171 | 331065.2 | 739 | 1128 | forward 1 | ICE-like protease (caspase) p20 domain | 9.40E−77 |
| 172 | 331065.5 | 180 | 446 | forward 3 | Caspase recruitment domain | 3.70E−32 |
| 172 | 331065.5 | 1143 | 1424 | forward 3 | ICE-like protease (caspase) p10 domain | 1.60E−50 |
| 172 | 331065.5 | 675 | 1064 | forward 3 | ICE-like protease (caspase) p20 domain | 1.50E−81 |
| 173 | 252782.4 | 644 | 793 | forward 2 | F-box domain. | 2.20E−04 |
| 176 | 200163.2 | 468 | 1289 | forward 3 | Cell division protein | 1.90E−119 |
| 178 | 1328237.4 | 780 | 1508 | forward 3 | Eukaryotic protein kinase domain | 4.60E−54 |
| 179 | 333965.1 | 1179 | 1979 | forward 3 | Eukaryotic protein kinase domain | 2.50E−59 |
| 179 | 333965.1 | 1980 | 2066 | forward 3 | Protein kinase C terminal domain | 2.30E−08 |
| 180 | 1328236.5 | 112 | 672 | forward 1 | Ras family | 5.90E−87 |
| 181 | 995534.3 | 686 | 811 | forward 2 | WD domain, G-beta repeat | 5.40E−05 |
| 182 | 1098887.1 | 325 | 612 | forward 1 | SCAN domain | 7.80E−70 |
| 182 | 1098887.1 | 1192 | 1260 | forward 1 | Zinc finger, C2H2 type | 4.60E−07 |
| 184 | 470023.2 | 456 | 815 | forward 3 | Serpins (serine protease inhibitors) | 2.40E−68 |
| 185 | 989966.8 | 424 | 1611 | forward 1 | N2,N2-dimethylguanosine tRNA methyltransferase | 2.60E−221 |
| 185 | 989966.8 | 1872 | 1958 | forward 3 | Zinc finger C-x8-C-x5-C-x3-H type (and similar). | 2.90E−04 |
| 186 | 233925.5 | 571 | 783 | forward 1 | RNA recognition motif. (a.k.a. RRM, RBD, or RNP domain) | 6.50E−20 |
| 187 | 405145.5 | 1112 | 1579 | forward 2 | GAF domain | 1.50E−28 |
| 187 | 405145.5 | 2456 | 3172 | forward 2 | 3'5'-cyclic nucleotide phosphodiesterase | 6.60E−96 |
| 188 | 261982.8 | 6390 | 6641 | forward 3 | Helicases conserved C-terminal domain | 2.00E−15 |
| 188 | 261982.8 | 4863 | 5843 | forward 3 | SNF2 and others N-terminal domain | 7.70E−139 |
| 193 | 898850.21 | 3205 | 3297 | forward 1 | Tau and MAP proteins, tubulin-binding | 1.60E−16 |
| 196 | 318000.4 | 225 | 755 | forward 3 | ADP-ribosylation factor family | 1.20E−39 |
| 196 | 318000.4 | 267 | 875 | forward 3 | Ras family | 7.20E−08 |
| 197 | 977887.1 | 2043 | 2825 | forward 3 | Uncharacterized protein family | 8.30E−97 |
| 198 | 1094199.1 | 513 | 707 | forward 3 | PAS domain | 1.50E−08 |
| 202 | 113621.5 | 89 | 625 | forward 2 | Tissue inhibitor of metalloproteinases | 1.20E−144 |
| 203 | 401532.2 | 1067 | 1882 | forward 2 | ABC transporter transmembrane region. | 4.40E−44 |
| 203 | 401532.2 | 4082 | 4633 | forward 2 | ABC transporter | 3.40E−46 |
| 207 | 475819.14 | 656 | 769 | forward 2 | Zinc finger, C3HC4 type (RING finger) | 1.80E−06 |
| 207 | 475819.14 | 656 | 769 | forward 2 | Zinc finger, C3HC4 type (RING finger) | 1.80E−06 |
| 208 | 241384.3 | 78 | 1982 | forward 3 | Glycosyl hydrolases family 35 | 0.00E+00 |
| 210 | 244200.1 | 394 | 948 | forward 1 | Glycosyl transferases | 2.40E−52 |
| 210 | 244200.1 | 1315 | 1692 | forward 1 | Similarity to lectin domain of ricin beta-chain, 3 copies. | 3.20E−45 |
| 214 | 903091.31 | 994 | 1131 | forward 1 | Zinc finger present in dystrophin, CBP/p300 | 3.30E−07 |
| 216 | 903091.16 | 876 | 1013 | forward 3 | Zinc finger present in dystrophin, CBP/p300 | 3.30E−07 |
| 218 | 475473.1 | 6003 | 6053 | forward 3 | Neuraxin and MAP1B proteins | 1.20E−08 |
| 223 | 903338.12 | 142 | 300 | forward 1 | Defensin propeptide | 3.00E−25 |
| 223 | 903338.12 | 337 | 423 | forward 1 | Mammalian defensin | 3.10E−14 |
| 225 | 1040667.52 | 1868 | 2074 | forward 2 | RNA recognition motif. (a.k.a. RRM, RBD, or RNP domain) | 2.40E−04 |
| 227 | 218524.4 | 596 | 1345 | forward 2 | 7 transmembrane receptor (rhodopsin family) | 7.10E−94 |
| 228 | 346673.1 | 533 | 727 | forward 2 | Double-stranded RNA binding motif | 2.60E−06 |
| 230 | 197086.1 | 1317 | 1460 | forward 3 | GRIP domain | 2.10E−07 |
| 231 | 348143.7 | 677 | 796 | forward 2 | UBA domain | 9.20E−13 |
| 231 | 348143.7 | 206 | 649 | forward 2 | Ubiquitin-conjugating enzyme | 5.50E−79 |
| 233 | 233003.6 | 509 | 604 | forward 2 | Ubiquitin carboxyl-terminal hydrolases family 2 | 8.60E−06 |
| 233 | 233003.6 | 1238 | 1504 | forward 2 | Ubiquitin carboxyl-terminal hydrolase family 2 | 3.30E−19 |
| 235 | 217860.1 | 559 | 900 | forward 1 | BTB/POZ domain | 4.40E−16 |
| 236 | 216189.2 | 431 | 898 | forward 2 | BTG1 family | 3.00E−103 |
| 238 | 382906.16 | 182 | 226 | forward 2 | Transmembrane 4 family | 4.40E−04 |
| 244 | 413835.5 | 250 | 633 | forward 1 | BTB/POZ domain | 3.60E−23 |
| 244 | 413835.5 | 1318 | 1386 | forward 1 | Zinc finger, C2H2 type | 7.80E−06 |
| 246 | 474673.1 | 4099 | 4554 | forward 1 | RhoGAP domain | 2.60E−54 |
| 247 | 480286.1 | 322 | 654 | forward 1 | Core histone H2A/H2B/H3/H4 | 2.50E−16 |
| 247 | 480286.1 | 347 | 679 | forward 2 | Core histone H2A/H2B/H3/H4 | 1.20E−14 |
| 252 | 253946.17 | 985 | 1254 | forward 1 | Fibronectin type III domain | 3.00E−14 |
| 261 | 82168.5 | 9273 | 9422 | forward 3 | Leucine rich repeat C-terminal domain | 6.40E−12 |
| 261 | 82168.5 | 9564 | 9980 | forward 3 | TIR domain | 6.60E−42 |
| 263 | 412661.2 | 269 | 463 | forward 2 | FHA domain | 4.30E−21 |
| 267 | 385608.2 | 251 | 991 | forward 2 | Transmembrane 4 family | 2.30E−160 |
| 279 | 82154.24 | 1684 | 1800 | forward 1 | WD domain, G-beta repeat | 7.70E−04 |
| 279 | 82154.24 | 1684 | 1800 | forward 1 | WD domain, G-beta repeat | 7.70E−04 |
| 283 | 90710.1 | 155 | 223 | forward 2 | Zinc finger, C2H2 type | 1.20E−04 |
| 290 | 350440.15 | 960 | 2684 | forward 3 | E1-E2 ATPase | 9.00E−59 |
| 290 | 350440.15 | 2599 | 2952 | forward 1 | E1-E2 ATPase | 8.60E−42 |
| 304 | 400280.3 | 600 | 803 | forward 3 | RNA recognition motif. (a.k.a. RRM, RBD, or RNP domain) | 3.80E−09 |
| 318 | 481847.1 | 202 | 318 | forward 1 | EGF-like domain | 5.20E−10 |
| 333 | 433569.1 | 450 | 1688 | forward 3 | Sulfatase | 4.70E−103 |
| 333 | 433569.1 | 260 | 1687 | forward 2 | Sulfatase | 5.50E−39 |
| 341 | 405041.1 | 435 | 533 | forward 3 | Ank repeat | 2.00E−08 |
| 342 | 474266.2 | 424 | 522 | forward 1 | Ank repeat | 2.30E−07 |
| 345 | 195199.1 | 886 | 984 | forward 1 | Ank repeat | 1.10E−04 |
| 348 | 256871.2 | 609 | 707 | forward 3 | Ank repeat | 2.00E−05 |
| 348 | 256871.2 | 609 | 707 | forward 3 | Ank repeat | 2.00E−05 |
| 356 | 344775.3 | 604 | 993 | forward 1 | SPRY domain | 1.80E−19 |
| 358 | 416874.3 | 313 | 558 | forward 1 | Kringle domain | 7.10E−37 |
| 358 | 416874.3 | 640 | 1362 | forward 1 | Trypsin | 1.70E−102 |
| 362 | 336953.7 | 2154 | 2882 | forward 3 | Biotin protein ligase | 2.30E−11 |
| 363 | 444771.2 | 2 | 352 | forward 2 | Elongation factor G C-terminus | 2.00E−04 |
| 364 | 449173.16 | 641 | 730 | forward2 | KH domain | 3.10E−04 |
| 367 | 252747.27 | 2298 | 2567 | forward 3 | C2 domain | 8.50E−17 |
| 369 | 1000084.27 | 152 | 1423 | forward 2 | Tubulin/FtsZ family | 2.40E−279 |
| 370 | 205607.5 | 1849 | 2733 | forward 1 | Phosphatidylinositol 3- and 4-kinases | 7.40E−116 |
| 370 | 205607.5 | 139 | 468 | forward 1 | C2 domain | 3.10E−44 |
| 370 | 205607.5 | 925 | 1668 | forward 1 | Phosphoinositide 3-kinase family, accessory domain (PIK dom | 2.10E−58 |
| 374 | 474724.5 | 340 | 780 | forward 1 | Eukaryotic protein kinase domain | 6.70E−25 |
| 394 | 238342.1 | 140 | 502 | forward 2 | BAH domain | 2.50E−16 |
| 411 | 411474.17 | 174 | 719 | forward 3 | ADP-ribosylation factor family | 7.60E−08 |
| 411 | 411474.17 | 231 | 815 | forward 3 | Ras family | 1.10E−112 |
| 415 | 263336.62 | 272 | 454 | forward 2 | Metallothionein | 2.10E−25 |
| 421 | 332919.4 | 703 | 801 | forward 1 | Ank repeat | 9.40E−08 |
| 425 | 987927.13 | 681 | 788 | forward 3 | EGF-like domain | 2.10E−04 |
| 427 | 1091415.2 | 775 | 1425 | forward 1 | Fibrinogen beta and gamma chains, C-terminal globular domai | 5.60E−63 |
| 430 | 444648.9 | 366 | 548 | forward 3 | Metallothionein | 1.20E−24 |
| 431 | 464689.22 | 608 | 1342 | forward 2 | Fatty acid desaturase | 1.20E−163 |
| 435 | 482336.11 | 95 | 310 | forward 2 | Intermediate filament proteins | 1.50E−21 |
| 442 | 210945.6 | 155 | 481 | forward 2 | FKBP-type peptidyl-prolyl cis-trans isomerases | 1.90E−52 |
| 448 | 345705.7 | 232 | 573 | forward 1 | BTB/POZ domain | 1.30E−28 |
| 448 | 345705.7 | 1525 | 1668 | forward 1 | Kelch motif | 2.50E−09 |
| 449 | 198212.1 | 816 | 986 | forward 3 | LIM domain containing proteins | 3.00E−15 |
| 450 | 198212.6 | 635 | 805 | forward 2 | LIM domain containing proteins | 3.00E−15 |
| 450 | 198212.6 | 277 | 453 | forward 1 | LIM domain containing proteins | 8.60E−07 |
| 450 | 198212.6 | 801 | 968 | forward 3 | LIM domain containing proteins | 2.10E−06 |
| 452 | 900031.4 | 2003 | 2101 | forward 2 | Ank repeat | 1.60E−05 |
| 452 | 900031.4 | 4406 | 4663 | forward 2 | Death domain | 9.40E−20 |
| 452 | 900031.4 | 509 | 1297 | forward 2 | Eukaryotic protein kinase domain | 4.60E−87 |
| 453 | 475365.4 | 607 | 1455 | forward 1 | Zinc carboxypeptidase | 1.30E−111 |
| 454 | 222810.1 | 384 | 965 | forward 3 | 7 transmembrane receptor (rhodopsin family) | 2.60E−34 |
| 454 | 222810.1 | 206 | 409 | forward 2 | 7 transmembrane receptor (rhodopsin family) | 1.00E−15 |
| 455 | 1084493.6 | 516 | 995 | forward 3 | Eukaryotic protein kinase domain | 1.00E−47 |
| 455 | 1084493.6 | 31 | 258 | forward 1 | Eukaryotic protein kinase domain | 2.70E−08 |
| 457 | 346686.23 | 1881 | 2129 | forward 3 | Fibronectin type III domain | 1.00E−06 |
| 462 | 243103.24 | 683 | 778 | forward 2 | EGF-like domain | 7.40E−09 |
| 463 | 469883.1 | 2253 | 2426 | forward 3 | Immunoglobulin domain | 5.20E−08 |
| 463 | 469883.1 | 2052 | 2210 | forward 3 | Plexin repeat | 4.80E−09 |
| 463 | 469883.1 | 696 | 1994 | forward 3 | Sema domain | 1.40E−207 |
| 466 | 903691.6 | 261 | 344 | forward 3 | Leucine rich repeat N-terminal domain | 5.60E−04 |
| 470 | 474630.19 | 4403 | 4657 | forward 2 | Fibronectin type III domain | 1.80E−25 |
| 470 | 474630.19 | 3493 | 3738 | forward 1 | Fibronectin type III domain | 3.70E−19 |
| 470 | 474630.19 | 264 | 1520 | forward 3 | Integrins, beta chain | 6.3e−317 |
| 472 | 1092445.1 | 399 | 686 | forward 3 | SCAN domain | 5.50E−58 |
| 472 | 1092445.1 | 1437 | 1505 | forward 3 | Zinc finger, C2H2 type | 2.40E−07 |
| 477 | 347005.5 | 361 | 603 | forward 1 | Fibronectin type III domain | 9.60E−18 |
| 477 | 347005.5 | 5206 | 5913 | forward 1 | Protein-tyrosine phosphatase | 1.50E−140 |
| 481 | 466402.116 | 657 | 1082 | forward 3 | Myelin basic protein | 9.50E−08 |
| 484 | 254107.1 | 1446 | 1553 | forward 3 | EGF-like domain | 3.60E−05 |
| 485 | 208328.1 | 58 | 273 | forward 1 | Ubiquitin family | 7.90E−09 |
| 489 | 336256.1 | 1478 | 1618 | forward 2 | Hemopexin | 2.60E−16 |
| 489 | 336256.1 | 347 | 901 | forward 2 | Matrixin | 1.10E−56 |
| 491 | 351432.3 | 2537 | 4858 | forward 2 | HCO3- transporter family | 0.00E+00 |
| 493 | 332595.1 | 320 | 616 | forward 2 | Cadherin domain | 1.20E−09 |
| 493 | 332595.1 | 1 | 261 | forward 1 | Cadherin domain | 7.60E−07 |
| 499 | 344582.19 | 375 | 497 | forward 3 | TNFR/NGFR cysteine-rich region | 6.80E−05 |
| 500 | 997526.4 | 5 | 202 | forward 2 | HMG (high mobility group) box | 8.20E−13 |

TABLE 3

| SEQ ID- | | | | | |
|---|---|---|---|---|---|
| NO | TEMPLATE ID | START | STOP | FRAME | DOMAIN |
| 1 | 441269.2 | 928 | 1008 | forward 1 | SP |
| 2 | 3161.5 | 571 | 651 | forward 1 | TM |
| 2 | 3161.5 | 1426 | 1506 | forward 1 | TM |
| 2 | 3161.5 | 388 | 471 | forward 1 | SP |
| 2 | 3161.5 | 382 | 468 | forward 1 | TM |
| 3 | 977955.7 | 174 | 263 | forward 3 | SP |
| 4 | 350754.2 | 651 | 749 | forward 3 | SP |
| 4 | 350754.2 | 270 | 362 | forward 3 | SP |
| 6 | 1099593.13 | 730 | 810 | forward 1 | SP |
| 6 | 1099593.13 | 2130 | 2210 | forward 3 | TM |
| 7 | 1099593.2 | 161 | 259 | forward 2 | SP |
| 11 | 347915.14 | 4742 | 4840 | forward 2 | SP |
| 12 | 997395.1 | 3240 | 3323 | forward 3 | SP |
| 12 | 997395.1 | 2100 | 2180 | forward 3 | SP |
| 12 | 997395.1 | 3187 | 3291 | forward 1 | SP |
| 13 | 332783.1 | 1164 | 1244 | forward 3 | TM |
| 13 | 332783.1 | 1434 | 1511 | forward 3 | TM |
| 14 | 422289.1 | 2247 | 2327 | forward 3 | TM |
| 14 | 422289.1 | 2059 | 2142 | forward 1 | SP |
| 15 | 899410.5 | 1077 | 1163 | forward 3 | TM |
| 15 | 899410.5 | 2158 | 2244 | forward 1 | SP |
| 15 | 899410.5 | 350 | 430 | forward 2 | SP |
| 16 | 404155.2 | 265 | 348 | forward 1 | TM |
| 19 | 345860.21 | 772 | 864 | forward 1 | SP |
| 23 | 474069.8 | 970 | 1050 | forward 1 | TM |
| 26 | 216331.1 | 1465 | 1551 | forward 1 | TM |
| 27 | 482517.3 | 1939 | 2019 | forward 1 | TM |
| 27 | 482517.3 | 148 | 228 | forward 1 | TM |
| 27 | 482517.3 | 350 | 424 | forward 2 | TM |
| 27 | 482517.3 | 1170 | 1253 | forward 3 | SP |
| 27 | 482517.3 | 1490 | 1564 | forward 2 | TM |
| 27 | 482517.3 | 1753 | 1833 | forward 1 | TM |
| 27 | 482517.3 | 300 | 377 | forward 3 | TM |
| 29 | 978047.1 | 1784 | 1861 | forward 2 | TM |
| 29 | 978047.1 | 1834 | 1914 | forward 1 | TM |
| 29 | 978047.1 | 1094 | 1180 | forward 2 | SP |
| 33 | 898068.6 | 446 | 532 | forward 2 | SP |
| 35 | 903104.8 | 1628 | 1717 | forward 2 | TM |
| 36 | 412065.21 | 1549 | 1635 | forward 1 | SP |
| 36 | 412065.21 | 1478 | 1579 | forward 2 | SP |
| 37 | 399067.1 | 2363 | 2446 | forward 2 | TM |
| 37 | 399067.1 | 109 | 189 | forward 1 | SP |
| 38 | 237709.11 | 301 | 384 | forward 1 | SP |
| 39 | 237709.5 | 766 | 849 | forward 1 | SP |
| 40 | 216437.4 | 432 | 527 | forward 3 | SP |
| 40 | 216437.4 | 479 | 559 | forward 2 | TM |
| 42 | 27798.1 | 57 | 137 | forward 3 | TM |
| 42 | 27798.1 | 2134 | 2226 | forward 1 | SP |
| 42 | 27798.1 | 1483 | 1566 | forward 1 | SP |
| 44 | 376085.9 | 656 | 751 | forward 2 | SP |
| 46 | 903508.12 | 2552 | 2632 | forward 2 | TM |
| 49 | 391406.5 | 463 | 546 | forward 1 | SP |
| 49 | 391406.5 | 2070 | 2153 | forward 3 | SP |
| 50 | 391406.24 | 208 | 291 | forward 1 | SP |
| 51 | 474588.21 | 1315 | 1392 | forward 1 | TM |
| 53 | 246862.9 | 834 | 932 | forward 3 | SP |
| 54 | 246862.17 | 1393 | 1473 | forward 1 | SP |
| 54 | 246862.17 | 934 | 1032 | forward 1 | SP |
| 54 | 246862.17 | 1573 | 1662 | forward 1 | SP |
| 57 | 314831.5 | 1070 | 1159 | forward 2 | SP |
| 58 | 391940.1 | 1112 | 1192 | forward 2 | SP |
| 58 | 391940.1 | 5869 | 5946 | forward 1 | TM |
| 58 | 391940.1 | 4958 | 5038 | forward 2 | SP |
| 58 | 391940.1 | 6060 | 6146 | forward 3 | SP |
| 59 | 454958.13 | 214 | 291 | forward 1 | SP |
| 61 | 238203.11 | 2656 | 2733 | forward 1 | SP |
| 61 | 238203.11 | 4781 | 4864 | forward 2 | TM |
| 61 | 238203.11 | 4074 | 4154 | forward 3 | TM |
| 62 | 994057.21 | 819 | 902 | forward 3 | SP |
| 62 | 994057.21 | 3427 | 3498 | forward 1 | TM |
| 62 | 994057.21 | 1776 | 1862 | forward 3 | TM |
| 66 | 482336.2 | 1051 | 1137 | forward 1 | SP |
| 66 | 482336.2 | 1702 | 1785 | forward 1 | SP |
| 67 | 482336.14 | 934 | 1020 | forward 1 | SP |
| 67 | 482336.14 | 1585 | 1668 | forward 1 | SP |
| 68 | 66522.1 | 294 | 380 | forward 3 | TM |
| 68 | 66522.1 | 31 | 108 | forward 1 | SP |
| 69 | 300294.3 | 1976 | 2053 | forward 2 | SP |
| 71 | 475028.7 | 1972 | 2061 | forward 1 | SP |
| 72 | 481223.3 | 2466 | 2561 | forward 3 | SP |
| 72 | 481223.3 | 1326 | 1406 | forward 3 | SP |
| 76 | 255778.11 | 1290 | 1376 | forward 3 | TM |
| 80 | 201342.4 | 528 | 608 | forward 3 | TM |
| 81 | 204542.1 | 9 | 92 | forward 3 | SP |
| 83 | 204386.1 | 1466 | 1549 | forward 2 | SP |
| 83 | 204386.1 | 1553 | 1642 | forward 2 | SP |
| 84 | 335086.1 | 2638 | 2718 | forward 1 | TM |
| 84 | 335086.1 | 376 | 462 | forward 1 | TM |
| 86 | 1063057.1 | 293 | 382 | forward 2 | SP |
| 87 | 199905.1 | 4765 | 4851 | forward 1 | TM |
| 87 | 199905.1 | 2052 | 2129 | forward 3 | SP |
| 87 | 199905.1 | 817 | 900 | forward 1 | SP |
| 87 | 199905.1 | 981 | 1052 | forward 3 | TM |
| 87 | 199905.1 | 236 | 340 | forward 2 | SP |
| 88 | 11329.1 | 185 | 265 | forward 2 | TM |
| 90 | 330878.6 | 2974 | 3066 | forward 1 | TM |
| 90 | 330878.6 | 2328 | 2405 | forward 3 | TM |
| 91 | 331793.11 | 3030 | 3107 | forward 3 | TM |
| 91 | 331793.11 | 139 | 219 | forward 1 | SP |
| 95 | 1095702.14 | 256 | 342 | forward 1 | SP |
| 97 | 977667.1 | 234 | 311 | forward 3 | TM |
| 101 | 382293.16 | 2167 | 2316 | forward 1 | SP |
| 101 | 382293.16 | 5189 | 5278 | forward 2 | SP |
| 101 | 382293.16 | 6113 | 6205 | forward 2 | SP |
| 101 | 382293.16 | 598 | 687 | forward 1 | SP |
| 101 | 382293.16 | 6017 | 6097 | forward 2 | SP |
| 101 | 382293.16 | 596 | 682 | forward 2 | SP |
| 102 | 382293.15 | 2423 | 2572 | forward 2 | SP |
| 102 | 382293.15 | 827 | 916 | forward 2 | SP |
| 102 | 382293.15 | 816 | 902 | forward 3 | SP |
| 104 | 235943.36 | 572 | 664 | forward 2 | SP |
| 104 | 235943.36 | 52 | 132 | forward 1 | SP |
| 106 | 234107.2 | 54 | 134 | forward 3 | SP |
| 108 | 482423.1 | 842 | 931 | forward 2 | SP |
| 108 | 482423.1 | 389 | 466 | forward 2 | SP |
| 109 | 441298.14 | 4843 | 4923 | forward 1 | SP |
| 109 | 441298.14 | 480 | 569 | forward 3 | SP |
| 109 | 441298.14 | 4439 | 4519 | forward 2 | SP |
| 112 | 992317.12 | 219 | 323 | forward 3 | SP |
| 113 | 230936.6 | 2690 | 2776 | forward 2 | TM |
| 113 | 230936.6 | 2433 | 2528 | forward 3 | SP |
| 115 | 984900.1 | 464 | 541 | forward 2 | TM |
| 116 | 405158.1 | 3164 | 3238 | forward 2 | TM |
| 116 | 405158.1 | 4889 | 4972 | forward 2 | SP |
| 116 | 405158.1 | 4318 | 4404 | forward 1 | TM |
| 116 | 405158.1 | 5463 | 5540 | forward 3 | TM |
| 120 | 1087696.7 | 56 | 142 | forward 2 | SP |
| 122 | 399128.1 | 27 | 116 | forward 3 | TM |
| 123 | 986565.15 | 1255 | 1335 | forward 1 | TM |
| 123 | 986565.15 | 2254 | 2331 | forward 1 | TM |
| 123 | 986565.15 | 1121 | 1204 | forward 2 | SP |
| 123 | 986565.15 | 2148 | 2225 | forward 3 | TM |
| 123 | 986565.15 | 2493 | 2570 | forward 3 | TM |
| 123 | 986565.15 | 1430 | 1522 | forward 2 | SP |
| 126 | 233594.4 | 613 | 699 | forward 1 | SP |
| 128 | 1080496.1 | 1278 | 1373 | forward 3 | SP |
| 129 | 351204.2 | 554 | 631 | forward 2 | SP |
| 129 | 351204.2 | 35 | 124 | forward 2 | SP |
| 132 | 427529.9 | 145 | 225 | forward 1 | SP |
| 133 | 474868.2 | 459 | 542 | forward 3 | SP |
| 133 | 474868.2 | 382 | 468 | forward 1 | SP |
| 134 | 1095839.17 | 406 | 501 | forward 1 | SP |
| 135 | 1095839.1 | 29 | 142 | forward 2 | SP |
| 135 | 1095839.1 | 1105 | 1182 | forward 1 | TM |
| 135 | 1095839.1 | 1813 | 1890 | forward 1 | TM |
| 136 | 244251.4 | 239 | 325 | forward 2 | SP |
| 136 | 244251.4 | 720 | 800 | forward 3 | SP |
| 136 | 244251.4 | 70 | 150 | forward 1 | TM |
| 136 | 244251.4 | 651 | 734 | forward 3 | TM |
| 138 | 234157.3 | 1914 | 2006 | forward 3 | SP |
| 142 | 407260.2 | 296 | 382 | forward 2 | SP |
| 145 | 231270.1 | 1138 | 1227 | forward 1 | SP |
| 148 | 404197.4 | 5001 | 5081 | forward 3 | SP |
| 148 | 404197.4 | 461 | 562 | forward 2 | SP |
| 148 | 404197.4 | 1523 | 1615 | forward 2 | SP |
| 150 | 346511.4 | 3324 | 3428 | forward 3 | SP |
| 152 | 331233.2 | 19 | 114 | forward 1 | SP |
| 154 | 238118.1 | 21 | 98 | forward 3 | TM |
| 155 | 233596.5 | 1762 | 1839 | forward 1 | TM |
| 155 | 233596.5 | 2332 | 2421 | forward 1 | SP |
| 156 | 988653.1 | 1080 | 1166 | forward 3 | SP |
| 156 | 988653.1 | 3422 | 3502 | forward 2 | TM |
| 157 | 470468.26 | 913 | 999 | forward 1 | SP |
| 159 | 903479.3 | 2576 | 2659 | forward 2 | TM |
| 159 | 903479.3 | 2571 | 2663 | forward 3 | TM |
| 159 | 903479.3 | 6877 | 6951 | forward 1 | TM |
| 159 | 903479.3 | 2017 | 2094 | forward 1 | TM |
| 159 | 903479.3 | 2563 | 2640 | forward 1 | TM |
| 159 | 903479.3 | 5437 | 5514 | forward 1 | TM |
| 159 | 903479.3 | 5549 | 5632 | forward 2 | TM |
| 161 | 1094412.1 | 179 | 259 | forward 2 | SP |
| 161 | 1094412.1 | 1565 | 1651 | forward 2 | SP |
| 162 | 1094107.1 | 179 | 259 | forward 2 | SP |
| 162 | 1094107.1 | 1510 | 1596 | forward 1 | SP |
| 162 | 1094107.1 | 669 | 794 | forward 3 | SP |
| 164 | 12178.2 | 387 | 461 | forward 3 | TM |
| 164 | 12178.2 | 430 | 510 | forward 1 | SP |
| 166 | 978478.4 | 791 | 862 | forward 2 | TM |
| 167 | 85942.2 | 285 | 371 | forward 3 | SP |
| 170 | 283762.2 | 4645 | 4725 | forward 1 | TM |
| 170 | 283762.2 | 4511 | 4600 | forward 2 | SP |
| 171 | 331065.2 | 72 | 161 | forward 3 | SP |
| 173 | 252782.4 | 3071 | 3154 | forward 2 | SP |
| 173 | 252782.4 | 1839 | 1922 | forward 3 | TM |
| 173 | 252782.4 | 1371 | 1451 | forward 3 | TM |
| 173 | 252782.4 | 1800 | 1883 | forward 3 | SP |
| 174 | 229068.1 | 648 | 731 | forward 3 | SP |
| 175 | 407450.7 | 1416 | 1502 | forward 3 | TM |
| 176 | 200163.2 | 367 | 450 | forward 1 | SP |
| 176 | 200163.2 | 3400 | 3474 | forward 1 | TM |
| 176 | 200163.2 | 3730 | 3828 | forward 1 | SP |
| 178 | 1328237.4 | 685 | 777 | forward 1 | SP |
| 178 | 1328237.4 | 2079 | 2159 | forward 3 | TM |
| 178 | 1328237.4 | 2006 | 2089 | forward 2 | TM |
| 178 | 1328237.4 | 2374 | 2451 | forward 1 | TM |
| 179 | 333965.1 | 1357 | 1440 | forward 1 | SP |
| 179 | 333965.1 | 435 | 542 | forward 3 | SP |
| 180 | 1328236.5 | 1014 | 1091 | forward 3 | TM |
| 181 | 995534.3 | 708 | 797 | forward 3 | SP |
| 184 | 470023.2 | 306 | 389 | forward 3 | SP |
| 186 | 233925.5 | 1436 | 1510 | forward 2 | TM |
| 186 | 233925.5 | 440 | 523 | forward 2 | SP |
| 186 | 233925.5 | 1901 | 1984 | forward 2 | TM |
| 186 | 233925.5 | 3519 | 3596 | forward 3 | TM |
| 187 | 405145.5 | 6884 | 6967 | forward 2 | TM |
| 187 | 405145.5 | 6924 | 7043 | forward 3 | SP |
| 187 | 405145.5 | 1491 | 1574 | forward 3 | SP |
| 188 | 261982.8 | 7984 | 8061 | forward 1 | TM |
| 188 | 261982.8 | 9033 | 9110 | forward 3 | TM |
| 188 | 261982.8 | 9430 | 9522 | forward 1 | TM |
| 188 | 261982.8 | 7748 | 7828 | forward 2 | TM |
| 189 | 246285.1 | 833 | 937 | forward 2 | SP |
| 190 | 228511.1 | 4994 | 5074 | forward 2 | TM |
| 190 | 228511.1 | 4777 | 4854 | forward 1 | SP |
| 190 | 228511.1 | 1557 | 1640 | forward 3 | TM |
| 191 | 40290.1 | 656 | 736 | forward 2 | TM |
| 193 | 898850.21 | 4370 | 4456 | forward 2 | TM |
| 193 | 898850.21 | 3429 | 3518 | forward 3 | SP |
| 194 | 201356.1 | 923 | 1006 | forward 2 | TM |
| 196 | 318000.4 | 2025 | 2102 | forward 3 | SP |
| 196 | 318000.4 | 1684 | 1764 | forward 1 | TM |
| 197 | 977887.1 | 1426 | 1536 | forward 1 | SP |
| 197 | 977887.1 | 3731 | 3820 | forward 2 | SP |
| 198 | 1094199.1 | 1516 | 1641 | forward 1 | SP |
| 198 | 1094199.1 | 4082 | 4174 | forward 2 | SP |
| 202 | 113621.5 | 1299 | 1382 | forward 3 | SP |
| 202 | 113621.5 | 17 | 94 | forward 2 | SP |
| 203 | 401532.2 | 3138 | 3251 | forward 3 | SP |
| 203 | 401532.2 | 5214 | 5291 | forward 3 | TM |
| 203 | 401532.2 | 1914 | 1991 | forward 3 | SP |
| 203 | 401532.2 | 1415 | 1498 | forward 2 | SP |
| 203 | 401532.2 | 3449 | 3532 | forward 2 | TM |
| 203 | 401532.2 | 4014 | 4103 | forward 3 | SP |
| 205 | 351209.14 | 256 | 336 | forward 1 | SP |
| 206 | 351209.16 | 2134 | 2223 | forward 1 | SP |
| 206 | 351209.16 | 285 | 368 | forward 3 | SP |
| 206 | 351209.16 | 266 | 352 | forward 2 | SP |
| 208 | 241384.3 | 1003 | 1098 | forward 1 | SP |
| 208 | 241384.3 | 45 | 146 | forward 3 | SP |
| 210 | 244200.1 | 2306 | 2389 | forward 2 | TM |
| 210 | 244200.1 | 2136 | 2219 | forward 3 | TM |
| 216 | 903091.16 | 1420 | 1506 | forward 1 | SP |
| 216 | 903091.16 | 1768 | 1854 | forward 1 | SP |
| 218 | 475473.1 | 5134 | 5223 | forward 1 | SP |
| 218 | 475473.1 | 7072 | 7152 | forward 1 | SP |
| 219 | 354430.4 | 4937 | 5020 | forward 2 | SP |
| 219 | 354430.4 | 6314 | 6409 | forward 2 | SP |
| 219 | 354430.4 | 469 | 546 | forward 1 | SP |
| 219 | 354430.4 | 5436 | 5540 | forward 3 | SP |
| 223 | 903338.12 | 115 | 198 | forward 1 | SP |
| 225 | 1040667.52 | 3266 | 3349 | forward 2 | TM |
| 225 | 1040667.52 | 666 | 749 | forward 3 | SP |
| 225 | 1040667.52 | 3309 | 3395 | forward 3 | SP |
| 225 | 1040667.52 | 510 | 590 | forward 3 | TM |
| 225 | 1040667.52 | 3408 | 3491 | forward 3 | SP |
| 227 | 218524.4 | 1130 | 1210 | forward 2 | SP |
| 227 | 218524.4 | 1350 | 1433 | forward 3 | SP |
| 227 | 218524.4 | 554 | 634 | forward 2 | TM |
| 229 | 58775.1 | 2 | 88 | forward 2 | SP |
| 231 | 348143.7 | 2697 | 2771 | forward 3 | TM |
| 231 | 348143.7 | 4440 | 4526 | forward 3 | TM |
| 231 | 348143.7 | 1859 | 1939 | forward 2 | TM |
| 233 | 233003.6 | 402 | 479 | forward 3 | TM |
| 236 | 216189.2 | 843 | 920 | forward 3 | SP |
| 236 | 216189.2 | 1332 | 1418 | forward 3 | SP |
| 236 | 216189.2 | 1035 | 1118 | forward 3 | SP |
| 238 | 382906.16 | 155 | 238 | forward 2 | SP |
| 241 | 346016.5 | 1841 | 1924 | forward 2 | SP |
| 241 | 346016.5 | 1921 | 2013 | forward 1 | SP |
| 241 | 346016.5 | 2054 | 2161 | forward 2 | SP |
| 241 | 346016.5 | 359 | 448 | forward 2 | SP |
| 242 | 346016.6 | 1836 | 1919 | forward 3 | SP |
| 242 | 346016.6 | 1799 | 1891 | forward 2 | SP |
| 242 | 346016.6 | 2049 | 2156 | forward 3 | SP |
| 242 | 346016.6 | 1737 | 1829 | forward 3 | SP |
| 242 | 346016.6 | 4777 | 4854 | forward 1 | SP |
| 244 | 413835.5 | 3440 | 3517 | forward 2 | SP |
| 246 | 474673.1 | 5054 | 5125 | forward 2 | TM |
| 246 | 474673.1 | 6072 | 6155 | forward 3 | TM |
| 246 | 474673.1 | 1883 | 1966 | forward 2 | TM |
| 246 | 474673.1 | 6379 | 6456 | forward 1 | TM |
| 246 | 474673.1 | 7446 | 7526 | forward 3 | TM |
| 246 | 474673.1 | 8881 | 8964 | forward 1 | TM |
| 248 | 402640.1 | 449 | 526 | forward 2 | TM |
| 248 | 402640.1 | 369 | 476 | forward 3 | SP |
| 252 | 253946.17 | 6747 | 6842 | forward 3 | SP |
| 252 | 253946.17 | 4350 | 4430 | forward 3 | TM |
| 252 | 253946.17 | 5326 | 5406 | forward 1 | TM |
| 252 | 253946.17 | 7207 | 7284 | forward 1 | TM |
| 252 | 253946.17 | 245 | 322 | forward 2 | SP |
| 254 | 997704.1 | 209 | 322 | forward 2 | SP |
| 261 | 82168.5 | 1162 | 1248 | forward 1 | SP |
| 261 | 82168.5 | 1456 | 1530 | forward 1 | TM |
| 261 | 82168.5 | 7638 | 7718 | forward 3 | TM |
| 261 | 82168.5 | 7016 | 7099 | forward 2 | SP |
| 263 | 412661.2 | 10202 | 10297 | forward 2 | SP |
| 263 | 412661.2 | 1842 | 1919 | forward 3 | SP |
| 264 | 206310.2 | 1856 | 1933 | forward 2 | SP |
| 266 | 385608.47 | 3643 | 3720 | forward 1 | TM |
| 267 | 385608.2 | 422 | 529 | forward 2 | SP |
| 267 | 385608.2 | 260 | 340 | forward 2 | TM |
| 267 | 385608.2 | 890 | 973 | forward 2 | TM |
| 267 | 385608.2 | 473 | 550 | forward 2 | TM |
| 269 | 978402.3 | 159 | 242 | forward 3 | SP |
| 270 | 232146.1 | 1274 | 1387 | forward 2 | SP |
| 273 | 239797.3 | 346 | 426 | forward 1 | SP |
| 276 | 427554.6 | 59 | 154 | forward 2 | SP |
| 276 | 427554.6 | 79 | 156 | forward 1 | TM |
| 278 | 82154.23 | 267 | 347 | forward 3 | SP |
| 280 | 1137293.16 | 146 | 226 | forward 2 | SP |
| 288 | 477387.7 | 661 | 744 | forward 1 | SP |
| 290 | 350440.15 | 1123 | 1203 | forward 1 | SP |
| 290 | 350440.15 | 3629 | 3703 | forward 2 | SP |
| 290 | 350440.15 | 3972 | 4052 | forward 3 | TM |
| 290 | 350440.15 | 3146 | 3226 | forward 2 | SP |
| 290 | 350440.15 | 3377 | 3460 | forward 2 | SP |
| 292 | 997395.4 | 509 | 589 | forward 2 | SP |
| 295 | 201436.3 | 1688 | 1771 | forward 2 | SP |
| 295 | 201436.3 | 1658 | 1735 | forward 2 | TM |
| 296 | 198947.1 | 422 | 502 | forward 2 | SP |
| 296 | 198947.1 | 451 | 543 | forward 1 | SP |
| 296 | 198947.1 | 231 | 308 | forward 3 | SP |
| 304 | 400280.3 | 4930 | 5010 | forward 1 | SP |
| 304 | 400280.3 | 766 | 855 | forward 1 | SP |
| 304 | 400280.3 | 6645 | 6722 | forward 3 | TM |
| 304 | 400280.3 | 134 | 217 | forward 2 | TM |
| 304 | 400280.3 | 137 | 217 | forward 2 | SP |
| 304 | 400280.3 | 871 | 972 | forward 1 | SP |
| 304 | 400280.3 | 2963 | 3046 | forward 2 | SP |
| 304 | 400280.3 | 4929 | 5012 | forward 3 | TM |
| 304 | 400280.3 | 606 | 695 | forward 3 | TM |
| 304 | 400280.3 | 5865 | 5951 | forward 3 | TM |
| 304 | 400280.3 | 2746 | 2820 | forward 1 | TM |
| 305 | 17320.1 | 1071 | 1151 | forward 3 | SP |
| 308 | 107405.1 | 305 | 382 | forward 2 | TM |
| 310 | 337024.3 | 1776 | 1859 | forward 3 | TM |
| 318 | 481847.1 | 122 | 214 | forward 2 | SP |
| 319 | 235636.1 | 426 | 506 | forward 3 | TM |
| 322 | 16760.1 | 139 | 225 | forward 1 | SP |
| 323 | 198522.1 | 684 | 764 | forward 3 | TM |
| 323 | 198522.1 | 297 | 380 | forward 3 | TM |
| 329 | 903269.4 | 1479 | 1562 | forward 3 | TM |
| 329 | 903269.4 | 4041 | 4148 | forward 3 | SP |
| 329 | 903269.4 | 180 | 266 | forward 3 | SP |
| 330 | 351166.1 | 183 | 257 | forward 3 | TM |
| 331 | 344713.3 | 168 | 254 | forward 3 | TM |
| 332 | 167920.1 | 26 | 109 | forward 2 | TM |
| 332 | 167920.1 | 188 | 271 | forward 2 | TM |
| 333 | 433569.1 | 970 | 1044 | forward 1 | SP |
| 333 | 433569.1 | 149 | 241 | forward 2 | SP |
| 335 | 348121.9 | 4210 | 4299 | forward 1 | SP |
| 341 | 405041.1 | 94 | 186 | forward 1 | SP |
| 342 | 474266.2 | 2672 | 2749 | forward 2 | TM |
| 342 | 474266.2 | 617 | 700 | forward 2 | SP |
| 342 | 474266.2 | 2725 | 2808 | forward 1 | TM |
| 347 | 372313.6 | 2350 | 2424 | forward 1 | TM |
| 348 | 256871.2 | 5 | 100 | forward 2 | SP |
| 348 | 256871.2 | 5 | 100 | forward 2 | SP |
| 350 | 22485.15 | 308 | 394 | forward 2 | SP |
| 350 | 22485.15 | 13 | 102 | forward 1 | SP |
| 351 | 22485.1 | 360 | 446 | forward 3 | SP |
| 351 | 22485.1 | 65 | 154 | forward 2 | SP |
| 352 | 26410.1 | 337 | 417 | forward 1 | TM |
| 354 | 472165.22 | 1863 | 1949 | forward 3 | SP |
| 355 | 206580.1 | 227 | 307 | forward 2 | TM |
| 358 | 416874.3 | 76 | 165 | forward 1 | SP |
| 364 | 449173.16 | 1573 | 1653 | forward 1 | TM |
| 367 | 252747.27 | 270 | 353 | forward 3 | SP |
| 369 | 1000084.27 | 4622 | 4705 | forward 2 | SP |
| 369 | 1000084.27 | 309 | 410 | forward 3 | SP |
| 369 | 1000084.27 | 1089 | 1169 | forward 3 | SP |
| 369 | 1000084.27 | 4170 | 4259 | forward 3 | SP |
| 369 | 1000084.27 | 4040 | 4123 | forward 2 | SP |
| 369 | 1000084.27 | 4138 | 4227 | forward 1 | SP |
| 370 | 205607.5 | 2447 | 2533 | forward 2 | SP |
| 370 | 205607.5 | 2060 | 2146 | forward 2 | SP |
| 371 | 1089426.1 | 625 | 714 | forward 1 | SP |
| 371 | 1089426.1 | 482 | 562 | forward 2 | SP |
| 374 | 474724.5 | 3145 | 3222 | forward 1 | TM |
| 374 | 474724.5 | 2538 | 2618 | forward 3 | TM |
| 374 | 474724.5 | 3103 | 3189 | forward 1 | SP |
| 374 | 474724.5 | 1154 | 1234 | forward 2 | TM |
| 374 | 474724.5 | 3544 | 3630 | forward 1 | TM |
| 377 | 229298.1 | 2450 | 2551 | forward 2 | SP |
| 377 | 229298.1 | 923 | 1015 | forward 2 | SP |
| 378 | 229298.2 | 28 | 114 | forward 1 | SP |
| 378 | 229298.2 | 128 | 220 | forward 2 | SP |
| 384 | 344398.2 | 1812 | 1889 | forward 3 | TM |
| 384 | 344398.2 | 2095 | 2172 | forward 1 | SP |
| 394 | 238342.1 | 1451 | 1534 | forward 2 | SP |
| 394 | 238342.1 | 2636 | 2716 | forward 2 | SP |
| 397 | 347865.4 | 584 | 664 | forward 2 | SP |
| 398 | 40057.2 | 1114 | 1209 | forward 1 | SP |
| 404 | 238071.2 | 3338 | 3415 | forward 2 | TM |
| 404 | 238071.2 | 969 | 1049 | forward 3 | SP |
| 404 | 238071.2 | 3274 | 3357 | forward 1 | TM |
| 404 | 238071.2 | 593 | 673 | forward 2 | SP |
| 404 | 238071.2 | 630 | 704 | forward 3 | TM |
| 404 | 238071.2 | 3301 | 3381 | forward 1 | SP |
| 407 | 221812.1 | 10 | 96 | forward 1 | TM |
| 410 | 347796.7 | 2467 | 2550 | forward 1 | TM |
| 411 | 411474.17 | 4552 | 4647 | forward 1 | SP |
| 416 | 153860.6 | 454 | 543 | forward 1 | SP |
| 417 | 154178.1 | 270 | 356 | forward 3 | SP |
| 418 | 337888.3 | 1716 | 1799 | forward 3 | TM |
| 418 | 337888.3 | 963 | 1040 | forward 3 | TM |
| 420 | 160011.1 | 419 | 505 | forward 2 | TM |
| 420 | 160011.1 | 447 | 530 | forward 3 | TM |
| 427 | 1091415.2 | 226 | 315 | forward 1 | SP |
| 431 | 464689.22 | 4918 | 4998 | forward 1 | TM |
| 434 | 351241.1 | 139 | 219 | forward 1 | TM |
| 436 | 482336.31 | 1147 | 1230 | forward 1 | SP |
| 437 | 197538.2 | 358 | 465 | forward 1 | SP |
| 438 | 197538.8 | 795 | 875 | forward 3 | TM |
| 442 | 210945.6 | 513 | 605 | forward 3 | SP |
| 442 | 210945.6 | 408 | 488 | forward 3 | SP |
| 444 | 403448.2 | 2437 | 2517 | forward 1 | TM |
| 444 | 403448.2 | 1994 | 2071 | forward 2 | TM |
| 444 | 403448.2 | 2034 | 2114 | forward 3 | TM |
| 446 | 146382.22 | 1394 | 1486 | forward 2 | SP |
| 447 | 146382.25 | 36 | 128 | forward 3 | SP |
| 448 | 345705.7 | 365 | 448 | forward 2 | SP |
| 448 | 345705.7 | 101 | 190 | forward 2 | SP |
| 452 | 900031.4 | 2733 | 2816 | forward 3 | SP |
| 452 | 900031.4 | 4841 | 4939 | forward 2 | SP |
| 452 | 900031.4 | 4494 | 4574 | forward 3 | SP |
| 453 | 475365.4 | 1519 | 1599 | forward 1 | SP |
| 454 | 222810.1 | 161 | 244 | forward 2 | TM |
| 455 | 1084493.6 | 2952 | 3044 | forward 3 | SP |
| 456 | 979005.2 | 1477 | 1557 | forward 1 | TM |
| 456 | 979005.2 | 2740 | 2820 | forward 1 | TM |
| 456 | 979005.2 | 1409 | 1486 | forward 2 | TM |
| 457 | 346686.23 | 2997 | 3080 | forward 3 | SP |
| 457 | 346686.23 | 1993 | 2076 | forward 1 | SP |
| 457 | 346686.23 | 2184 | 2258 | forward 3 | TM |
| 457 | 346686.23 | 303 | 389 | forward 3 | SP |
| 457 | 346686.23 | 2151 | 2240 | forward 3 | SP |
| 458 | 234568.25 | 202 | 285 | forward 1 | SP |
| 459 | 234568.17 | 1841 | 1933 | forward 2 | SP |
| 459 | 234568.17 | 2059 | 2136 | forward 1 | TM |
| 459 | 234568.17 | 208 | 291 | forward 1 | SP |
| 462 | 243103.24 | 164 | 241 | forward 2 | SP |
| 462 | 243103.24 | 588 | 674 | forward 3 | SP |
| 463 | 469883.1 | 2657 | 2734 | forward 2 | SP |
| 463 | 469883.1 | 4492 | 4572 | forward 1 | SP |
| 463 | 469883.1 | 4587 | 4670 | forward 3 | SP |
| 463 | 469883.1 | 1198 | 1287 | forward 1 | SP |
| 463 | 469883.1 | 1732 | 1821 | forward 1 | SP |
| 463 | 469883.1 | 4352 | 4435 | forward 2 | TM |
| 463 | 469883.1 | 3980 | 4060 | forward 2 | SP |
| 463 | 469883.1 | 2724 | 2813 | forward 3 | SP |
| 463 | 469883.1 | 400 | 486 | forward 1 | SP |
| 465 | 227932.2 | 4290 | 4370 | forward 3 | TM |
| 465 | 227932.2 | 3078 | 3155 | forward 3 | TM |
| 466 | 903691.6 | 195 | 278 | forward 3 | SP |
| 466 | 903691.6 | 2840 | 2929 | forward 2 | SP |
| 468 | 407938.5 | 3676 | 3756 | forward 1 | TM |
| 469 | 233778.1 | 13 | 93 | forward 1 | SP |
| 469 | 233778.1 | 371 | 451 | forward 2 | SP |
| 470 | 474630.19 | 2277 | 2369 | forward 3 | SP |
| 470 | 474630.19 | 156 | 236 | forward 3 | SP |
| 474 | 228860.6 | 1009 | 1098 | forward 1 | SP |
| 477 | 347005.5 | 755 | 844 | forward 2 | SP |
| 477 | 347005.5 | 524 | 607 | forward 2 | TM |
| 477 | 347005.5 | 1931 | 2017 | forward 2 | SP |
| 477 | 347005.5 | 4970 | 5053 | forward 2 | SP |
| 480 | 252570.6c | 215 | 295 | forward 2 | SP |
| 481 | 466402.116 | 1085 | 1177 | forward 2 | SP |
| 483 | 899612.2 | 393 | 479 | forward 3 | SP |
| 484 | 254107.1 | 1087 | 1170 | forward 1 | SP |
| 484 | 254107.1 | 2929 | 3054 | forward 1 | SP |
| 484 | 254107.1 | 3266 | 3349 | forward 2 | SP |
| 485 | 208328.1 | 2434 | 2517 | forward 1 | SP |
| 485 | 208328.1 | 2158 | 2244 | forward 1 | SP |
| 486 | 130157.2 | 1344 | 1421 | forward 3 | TM |
| 486 | 130157.2 | 1994 | 2074 | forward 2 | TM |
| 486 | 130157.2 | 2041 | 2121 | forward 1 | TM |
| 488 | 233089.1 | 1707 | 1787 | forward 3 | TM |
| 489 | 336256.1 | 2057 | 2140 | forward 2 | SP |
| 489 | 336256.1 | 1835 | 1918 | forward 2 | SP |
| 489 | 336256.1 | 3340 | 3420 | forward 1 | TM |
| 489 | 336256.1 | 236 | 319 | forward 2 | SP |
| 490 | 979616.2 | 319 | 393 | forward 1 | TM |
| 491 | 351432.3 | 4887 | 4964 | forward 3 | SP |
| 494 | 481251.3 | 1966 | 2043 | forward 1 | TM |
| 494 | 481251.3 | 1694 | 1774 | forward 2 | SP |
| 494 | 481251.3 | 1825 | 1908 | forward 1 | TM |
| 496 | 96700.1 | 141 | 218 | forward 3 | TM |
| 499 | 344582.19 | 161 | 247 | forward 2 | SP |

TABLE 4

| SEQ ID NO | TEMPLATE ID | CLONE ID | START | STOP |
|---|---|---|---|---|
| 1 | 441269.2 | 467767H1 | 1083 | 1317 |
| 1 | 441269.2 | 467767R6 | 1083 | 1491 |
| 1 | 441269.2 | 467767T6 | 1523 | 2042 |
| 2 | 3161.5 | 897743R6 | 2385 | 2856 |
| 2 | 3161.5 | 897743H1 | 2385 | 2549 |
| 3 | 977955.7 | 908504T6 | 3163 | 3619 |
| 3 | 977955.7 | 908504H1 | 549 | 748 |
| 3 | 977955.7 | 908504R6 | 549 | 947 |
| 4 | 350754.2 | 981592R6 | 1400 | 1980 |
| 4 | 350754.2 | 981592H1 | 1400 | 1658 |
| 4 | 350754.2 | 981592T6 | 2173 | 2701 |
| 5 | 235191.4 | 1308478H1 | 568 | 829 |
| 5 | 235191.4 | 1308478F6 | 568 | 829 |
| 5 | 235191.4 | 1308478T6 | 570 | 791 |
| 5 | 235191.4 | 1308478R1 | 656 | 844 |
| 6 | 1099593.13 | 1435427F6 | 720 | 1228 |
| 6 | 1099593.13 | 1435427H1 | 720 | 988 |
| 6 | 1099593.13 | 1435427F1 | 720 | 1227 |
| 7 | 1099593.2 | 1435427T6 | 692 | 977 |
| 8 | 003161.7c | 1484773F6 | 270 | 603 |
| 8 | 003161.7c | 1484773T6 | 271 | 578 |
| 8 | 003161.7c | 1484773H1 | 270 | 468 |
| 9 | 415650.5 | 1485735F6 | 80 | 588 |
| 9 | 415650.5 | 1485735H1 | 80 | 337 |
| 10 | 204401.1 | 1485735T6 | 8 | 579 |
| 11 | 347915.14 | 1503076T6 | 4750 | 5381 |
| 11 | 347915.14 | 1503076H1 | 4558 | 4847 |
| 11 | 347915.14 | 1503076F6 | 4558 | 5053 |
| 12 | 997395.1 | 1559836T6 | 2134 | 2777 |
| 12 | 997395.1 | 1559836H1 | 1946 | 2154 |
| 12 | 997395.1 | 1559836F6 | 1946 | 2323 |
| 13 | 332783.1 | 2450712F6 | 1161 | 1558 |
| 13 | 332783.1 | 2450712H1 | 1161 | 1400 |
| 13 | 332783.1 | 1612714F6 | 1653 | 2118 |
| 13 | 332783.1 | 1612714H1 | 1653 | 1866 |
| 13 | 332783.1 | 1612714T6 | 2047 | 2587 |
| 13 | 332783.1 | 2450712T6 | 2156 | 2648 |
| 14 | 422289.1 | 1676442H1 | 1178 | 1414 |
| 14 | 422289.1 | 1676442F6 | 1178 | 1661 |
| 14 | 422289.1 | 1676442T6 | 2168 | 2501 |
| 15 | 899410.5 | 1724967F6 | 920 | 1312 |
| 15 | 899410.5 | 1724967H1 | 1080 | 1312 |
| 16 | 404155.2 | 1724967T6 | 91 | 570 |
| 17 | 412065.22 | 2591494T6 | 1041 | 1681 |
| 17 | 412065.22 | 2591494F6 | 763 | 1349 |
| 17 | 412065.22 | 1737926F6 | 530 | 663 |
| 17 | 412065.22 | 1737926T6 | 1241 | 1679 |
| 18 | 412065.2 | 1737926H1 | 1 | 217 |
| 19 | 345860.21 | 1749417H1 | 309 | 624 |
| 19 | 345860.21 | 1749417F6 | 309 | 772 |
| 19 | 345860.21 | 1749417T6 | 1348 | 1440 |
| 20 | 199101.1 | 1758055H1 | 1553 | 1823 |
| 20 | 199101.1 | 1758055R6 | 1553 | 2065 |
| 20 | 199101.1 | 1758055T6 | 2470 | 2920 |
| 21 | 196606.8c | 1806542H1 | 2386 | 2681 |
| 21 | 196606.8c | 1806542F6 | 2116 | 2681 |
| 21 | 196606.8c | 1806542T6 | 289 | 780 |
| 22 | 475146.3 | 1807407T6 | 1489 | 1940 |
| 22 | 475146.3 | 1807407F6 | 466 | 1006 |
| 22 | 475146.3 | 1807407H1 | 466 | 714 |
| 23 | 474069.8 | 1811369H1 | 844 | 1150 |
| 23 | 474069.8 | 1811369F6 | 844 | 1283 |
| 23 | 474069.8 | 1811369T6 | 1722 | 2152 |
| 24 | 255115.2 | 1846973H1 | 865 | 1117 |
| 25 | 255115.4 | 1846973T6 | 1381 | 1829 |
| 26 | 216331.1 | 1869130H1 | 1226 | 1502 |
| 26 | 216331.1 | 1869130F6 | 1226 | 1653 |
| 26 | 216331.1 | 1869130T6 | 1406 | 1976 |
| 27 | 482517.3 | 1880840H1 | 852 | 999 |
| 27 | 482517.3 | 1880840F6 | 852 | 1289 |
| 27 | 482517.3 | 1880840T6 | 1593 | 2091 |
| 28 | 480653.1 | 1901284F6 | 111 | 569 |
| 28 | 480653.1 | 1901284H1 | 111 | 367 |
| 28 | 480653.1 | 1901284T6 | 666 | 1201 |
| 29 | 978047.1 | 1922875T6 | 1398 | 1987 |
| 29 | 978047.1 | 1922875H1 | 846 | 1029 |
| 29 | 978047.1 | 1922875R6 | 847 | 1347 |
| 30 | 330977.1c | 1998075H1 | 821 | 1095 |
| 30 | 330977.1c | 1998075R6 | 706 | 1095 |
| 30 | 330977.1c | 1998075T6 | 676 | 1068 |
| 31 | 343502.9 | 1998428H1 | 617 | 889 |
| 31 | 343502.9 | 1998428R6 | 617 | 1062 |
| 31 | 343502.9 | 1998428T6 | 617 | 974 |
| 32 | 903104.11c | 2054053R6 | 514 | 1051 |
| 32 | 903104.11c | 2054053H1 | 774 | 1051 |
| 33 | 898068.6 | 2054053T6 | 748 | 1276 |
| 34 | 903104.1 | 2263572R6 | 112 | 583 |
| 34 | 903104.1 | 2263572H1 | 95 | 346 |
| 35 | 903104.8 | 2054053CB1 | 2 | 2370 |
| 35 | 903104.8 | 2263572T6 | 1786 | 2241 |
| 36 | 412065.21 | 2591494H1 | 1010 | 1179 |
| 37 | 399067.1 | 2615184CA2 | 1 | 2855 |
| 37 | 399067.1 | 2615184H1 | 1 | 251 |
| 37 | 399067.1 | 2615184F6 | 1 | 380 |
| 37 | 399067.1 | 2615184T6 | 2108 | 2557 |
| 38 | 237709.11 | 2625371F6 | 1145 | 1704 |
| 38 | 237709.11 | 2625371T6 | 1166 | 1788 |
| 39 | 237709.5 | 2625371H1 | 1732 | 2007 |
| 40 | 216437.4 | 2636043CB1 | 98 | 1101 |
| 40 | 216437.4 | 2636043H1 | 998 | 1085 |
| 40 | 216437.4 | 2636043F6 | 492 | 1085 |
| 41 | 255824.52 | 2636043T6 | 1552 | 1836 |
| 42 | 27798.1 | 2653855T6 | 3882 | 4203 |
| 42 | 27798.1 | 2653855H1 | 2809 | 3102 |
| 42 | 27798.1 | 2653855F6 | 2809 | 3399 |
| 43 | 60671.7 | 2676441F6 | 584 | 989 |
| 43 | 60671.7 | 2676441H1 | 584 | 829 |
| 43 | 60671.7 | 2582525T6 | 1814 | 2355 |
| 44 | 376085.9 | 2676441T6 | 1998 | 2206 |
| 45 | 404467.1 | 2715440H1 | 1 | 242 |
| 45 | 404467.1 | 2715440F6 | 1 | 530 |
| 46 | 903508.12 | 2715440T6 | 2750 | 2967 |
| 47 | 334387.1 | 2718810T6 | 2277 | 2764 |
| 47 | 334387.1 | 2718810CB1 | 1 | 2834 |
| 47 | 334387.1 | 2718810F6 | 1239 | 1742 |
| 47 | 334387.1 | 2718810H1 | 1239 | 1496 |
| 48 | 290403.5 | 2839245F6 | 1055 | 1548 |
| 48 | 290403.5 | 2839245H1 | 1055 | 1319 |
| 49 | 391406.5 | 2883195CB1 | 1848 | 2974 |
| 49 | 391406.5 | 2883195F6 | 1912 | 2425 |
| 50 | 391406.24 | 2883195H1 | 4 | 277 |
| 50 | 391406.24 | 2883195T6 | 330 | 925 |
| 51 | 474588.21 | 3070625H1 | 1762 | 2041 |
| 51 | 474588.21 | 3070625F6 | 1761 | 2181 |
| 52 | 124541.1 | 3176339F6 | 3891 | 4412 |
| 52 | 124541.1 | 3176339T6 | 8 | 409 |
| 52 | 124541.1 | 3176339H1 | 3894 | 4135 |
| 53 | 246862.9 | 3206210F6 | 46 | 466 |
| 53 | 246862.9 | 3206210H1 | 46 | 149 |
| 54 | 246862.17 | 3206210T6 | 1519 | 1947 |
| 55 | 13040.1 | 3436305F6 | 106 | 357 |
| 55 | 13040.1 | 3436305H1 | 106 | 219 |
| 56 | 236480.3 | 3436305T6 | 3031 | 3443 |
| 57 | 314831.5 | 135769H1 | 1362 | 1577 |
| 57 | 314831.5 | 135769R6 | 1362 | 1874 |
| 57 | 314831.5 | 135769T6 | 1984 | 2532 |
| 58 | 391940.1 | 343763R6 | 2988 | 3443 |
| 58 | 391940.1 | 343763H1 | 2989 | 3091 |
| 58 | 391940.1 | 343763T6 | 3132 | 3646 |
| 59 | 454958.13 | 591358R1 | 582 | 929 |
| 59 | 454958.13 | 591358H1 | 582 | 729 |
| 59 | 454958.13 | 591358R6 | 582 | 938 |
| 60 | 1090481.2c | 591358T6 | 1 | 252 |
| 61 | 238203.11 | 999864R6 | 4229 | 4728 |
| 61 | 238203.11 | 999864H1 | 4229 | 4313 |
| 61 | 238203.11 | 999864T6 | 4548 | 5093 |
| 62 | 994057.21 | 1749663H1 | 1576 | 1716 |
| 62 | 994057.21 | 1749663F6 | 1576 | 2017 |
| 63 | 346730.5 | 1818365H1 | 548 | 841 |
| 63 | 346730.5 | 1818365F6 | 548 | 1047 |
| 64 | 346730.2c | 1818365T6 | 1 | 563 |
| 65 | 458903.1 | 1962141H1 | 353 | 588 |
| 66 | 482336.2 | 1962141R6 | 907 | 1438 |
| 67 | 482336.14 | 1962141T6 | 1320 | 1926 |
| 68 | 66522.1 | 1965083H1 | 1 | 266 |
| 68 | 66522.1 | 1965083R6 | 1 | 410 |
| 69 | 300294.3 | 1984158H1 | 2131 | 2393 |
| 69 | 300294.3 | 1984158R6 | 2131 | 2454 |
| 69 | 300294.3 | 1984158T6 | 2131 | 2429 |
| 70 | 474372.8 | 1988774R6 | 2510 | 2848 |
| 70 | 474372.8 | 1988774H1 | 2510 | 2780 |
| 71 | 475028.7 | 2057432CB1 | 1 | 2100 |
| 71 | 475028.7 | 2057432H1 | 140 | 427 |
| 71 | 475028.7 | 2057432R6 | 140 | 704 |
| 72 | 481223.3 | 2057432T6 | 2099 | 2683 |
| 73 | 332499.1 | 2123889T6 | 2129 | 2313 |
| 73 | 332499.1 | 2123889F6 | 2129 | 2350 |
| 73 | 332499.1 | 2123889H1 | 2129 | 2350 |
| 74 | 234340.7 | 2189132F6 | 17 | 458 |
| 74 | 234340.7 | 2189132H1 | 17 | 300 |
| 75 | 234340.15c | 2189132T6 | 60 | 513 |
| 76 | 255778.11 | 2374121H1 | 438 | 632 |
| 76 | 255778.11 | 2374121T6 | 1352 | 1841 |
| 76 | 255778.11 | 2374121CB1 | 1 | 958 |
| 76 | 255778.11 | 2374121F6 | 438 | 958 |
| 76 | 255778.11 | 2374121CA2 | 438 | 958 |
| 77 | 317586.1 | 2613155T6 | 1232 | 1666 |
| 77 | 317586.1 | 2613155F6 | 1513 | 1705 |
| 77 | 317586.1 | 2613155H1 | 1531 | 1705 |
| 78 | 1093574.1c | 2648611F6 | 607 | 1089 |
| 78 | 1093574.1c | 2648611H1 | 607 | 841 |
| 79 | 355658.1 | 2648611T6 | 1 | 233 |
| 80 | 201342.4 | 2802809T6 | 2155 | 2700 |
| 80 | 201342.4 | 2802809H1 | 1365 | 1634 |
| 80 | 201342.4 | 2802809F6 | 1365 | 1793 |
| 81 | 204542.1 | 2817637H1 | 2 | 266 |
| 82 | 428742.1 | 2817637T6 | 1 | 339 |
| 83 | 204386.1 | 3296181T6 | 2265 | 2823 |
| 83 | 204386.1 | 3296181H1 | 293 | 493 |
| 83 | 204386.1 | 3296181F6 | 293 | 698 |
| 84 | 335086.1 | 3602403H1 | 3018 | 3210 |
| 84 | 335086.1 | 3602403F6 | 3018 | 3387 |
| 85 | 228302.1 | 3602403T6 | 634 | 706 |
| 86 | 1063057.1 | 4140129T6 | 192 | 718 |
| 86 | 1063057.1 | 4140129H1 | 1 | 295 |
| 86 | 1063057.1 | 4140129F6 | 1 | 450 |
| 87 | 199905.1 | 384911H1 | 1883 | 2162 |
| 87 | 199905.1 | 384911R6 | 1888 | 2281 |
| 88 | 11329.1 | 501563T6 | 404 | 670 |
| 88 | 11329.1 | 501563H1 | 404 | 563 |
| 88 | 11329.1 | 501563R6 | 404 | 707 |
| 89 | 237536.18 | 549297H1 | 735 | 1047 |
| 89 | 237536.18 | 549297R6 | 766 | 1047 |
| 89 | 237536.18 | 549297T6 | 296 | 785 |
| 90 | 330878.6 | 642320R6 | 1556 | 2014 |
| 90 | 330878.6 | 642320H1 | 1556 | 1813 |
| 90 | 330878.6 | 642320T6 | 3156 | 3745 |
| 91 | 331793.11 | 737335CA2 | 112 | 1166 |
| 91 | 331793.11 | 737335H1 | 112 | 339 |
| 91 | 331793.11 | 737335R6 | 112 | 470 |
| 91 | 331793.11 | 737335T6 | 549 | 1116 |
| 92 | 902895.2 | 1234126F1 | 409 | 724 |
| 92 | 902895.2 | 1234126F6 | 409 | 724 |
| 92 | 902895.2 | 1234126H1 | 409 | 660 |
| 92 | 902895.2 | 1234126T6 | 409 | 674 |
| 93 | 7273.1 | 1367516R1 | 91 | 519 |
| 93 | 7273.1 | 1367516H1 | 91 | 228 |
| 93 | 7273.1 | 1367516R6 | 91 | 518 |
| 93 | 7273.1 | 1367516T6 | 171 | 565 |
| 94 | 190771.2 | 1394614F6 | 729 | 1173 |
| 94 | 190771.2 | 1394614H1 | 729 | 996 |
| 94 | 190771.2 | 1394614T6 | 1986 | 2563 |
| 95 | 1095702.14 | 1428779CB1 | 94 | 2366 |
| 95 | 1095702.14 | 1428779H1 | 111 | 360 |
| 95 | 1095702.14 | 1428779F6 | 111 | 406 |
| 96 | 1095702.4c | 1428779T6 | 5160 | 5336 |
| 97 | 977667.1 | 1446659H1 | 537 | 786 |
| 97 | 977667.1 | 1446659F6 | 537 | 979 |
| 97 | 977667.1 | 1446659T6 | 32 | 96 |
| 98 | 253534.14 | 1450849T6 | 1 | 417 |
| 98 | 253534.14 | 1450849H1 | 1 | 135 |
| 98 | 253534.14 | 1450849F6 | 1 | 443 |
| 98 | 253534.14 | 1450849H6 | 1 | 218 |
| 99 | 1136709.5 | 1488704H1 | 1 | 199 |
| 99 | 1136709.5 | 1488704CA2 | 1 | 768 |
| 99 | 1136709.5 | 1488704F6 | 1 | 511 |
| 100 | 1136709.6 | 1488704T6 | 116 | 578 |
| 101 | 382293.16 | 1599046H1 | 258 | 475 |
| 101 | 382293.16 | 1599046F6 | 258 | 655 |
| 102 | 382293.15 | 1599046T6 | 2489 | 2898 |
| 103 | 235943.39c | 1603809F6 | 2213 | 2678 |
| 103 | 235943.39c | 1603809CA2 | 1307 | 2678 |
| 104 | 235943.36 | 1603809H1 | 4 | 224 |
| 105 | 401434.1 | 1610682T6 | 1024 | 1310 |
| 105 | 401434.1 | 1610682H1 | 1031 | 1225 |
| 105 | 401434.1 | 1610682F6 | 1031 | 1351 |
| 106 | 234107.2 | 1615959T6 | 1788 | 2370 |
| 106 | 234107.2 | 1615959H1 | 695 | 885 |
| 106 | 234107.2 | 1615959F6 | 695 | 1056 |
| 107 | 233660.2c | 1626152F6 | 4392 | 4768 |
| 107 | 233660.2c | 1626152H1 | 4558 | 4768 |
| 107 | 233660.2c | 1626152T6 | 2831 | 3411 |
| 108 | 482423.1 | 1648841H1 | 696 | 856 |
| 108 | 482423.1 | 1648841F6 | 696 | 1033 |
| 109 | 441298.14 | 1676389T6 | 4775 | 5183 |
| 109 | 441298.14 | 1676389H1 | 3121 | 3333 |
| 109 | 441298.14 | 1676389F6 | 3121 | 3619 |
| 110 | 235333.1 | 1684632H1 | 739 | 945 |
| 110 | 235333.1 | 1684632F6 | 739 | 1215 |
| 110 | 235333.1 | 1684632T6 | 835 | 1320 |
| 111 | 221872.11c | 1722234F6 | 799 | 1068 |
| 111 | 221872.11c | 1722234H1 | 860 | 1068 |
| 112 | 992317.12 | 1725322T6 | 864 | 1342 |
| 112 | 992317.12 | 1725322F6 | 866 | 1269 |
| 112 | 992317.12 | 1725322H1 | 866 | 1082 |
| 113 | 230936.6 | 1747818T6 | 1813 | 2350 |
| 113 | 230936.6 | 1747818F6 | 2170 | 2729 |
| 113 | 230936.6 | 1747818H1 | 2462 | 2729 |
| 114 | 029508.1c | 1798468F6 | 97 | 539 |
| 114 | 029508.1c | 1798468H1 | 99 | 370 |
| 115 | 984900.1 | 1802185H1 | 29 | 180 |
| 115 | 984900.1 | 1802185F6 | 29 | 322 |
| 116 | 405158.1 | 1806433F6 | 1972 | 2176 |
| 116 | 405158.1 | 1806433H1 | 2019 | 2240 |
| 116 | 405158.1 | 1806433T6 | 2785 | 3209 |
| 117 | 334177.1 | 1849820H1 | 978 | 1232 |
| 117 | 334177.1 | 1849820F6 | 978 | 1434 |
| 118 | 480187.81 | 1870759CB1 | 1 | 1386 |
| 118 | 480187.81 | 1870759F6 | 36 | 467 |
| 118 | 480187.81 | 1870759H1 | 36 | 299 |
| 119 | 62042.4 | 1979528H1 | 1040 | 1286 |
| 119 | 62042.4 | 1979528R6 | 1040 | 1419 |
| 119 | 62042.4 | 1979528T6 | 1075 | 1534 |
| 120 | 1087696.7 | 2076621H1 | 10 | 273 |
| 120 | 1087696.7 | 2076621F6 | 10 | 428 |
| 121 | 259805.28 | 2076621T6 | 2555 | 2881 |
| 122 | 399128.1 | 2097461H1 | 1 | 225 |
| 122 | 399128.1 | 2097461R6 | 2 | 483 |
| 123 | 986565.15 | 2191455F6 | 603 | 936 |
| 123 | 986565.15 | 2191455H1 | 603 | 853 |
| 123 | 986565.15 | 2191455T6 | 3044 | 3532 |
| 124 | 978378.3 | 2202952H1 | 700 | 934 |
| 124 | 978378.3 | 2202952F6 | 700 | 1002 |
| 125 | 979575.2c | 2243954F6 | 672 | 1113 |
| 125 | 979575.2c | 2243954H1 | 862 | 1113 |
| 126 | 233594.4 | 2264081R6 | 61 | 348 |
| 126 | 233594.4 | 2264081H1 | 61 | 317 |
| 127 | 238635.1 | 2365066H1 | 232 | 464 |
| 127 | 238635.1 | 2365066F6 | 232 | 774 |
| 127 | 238635.1 | 2365066T6 | 466 | 882 |
| 128 | 1080496.1 | 2414663F6 | 599 | 1131 |
| 128 | 1080496.1 | 2414663H1 | 599 | 823 |
| 129 | 351204.2 | 2414663T6 | 702 | 990 |
| 130 | 235394.5 | 2447071F6 | 2469 | 2875 |
| 130 | 235394.5 | 2447071H1 | 2469 | 2686 |
| 131 | 403242.1 | 2500915F6 | 1724 | 2064 |
| 131 | 403242.1 | 2500915H1 | 1724 | 1949 |
| 131 | 403242.1 | 2500915T6 | 1883 | 2304 |
| 132 | 427529.9 | 2504404F6 | 22 | 511 |
| 132 | 427529.9 | 2504404H1 | 22 | 262 |
| 133 | 474868.2 | 2525691T6 | 1904 | 2376 |
| 133 | 474868.2 | 2525691F6 | 95 | 512 |
| 133 | 474868.2 | 2525691H1 | 95 | 360 |
| 133 | 474868.2 | 2525691CA2 | 100 | 2457 |
| 133 | 474868.2 | 2525691CB1 | 102 | 2450 |
| 134 | 1095839.17 | 2541141F6 | 44 | 531 |
| 134 | 1095839.17 | 2541141H1 | 44 | 276 |
| 135 | 1095839.1 | 2541141T6 | 3113 | 3589 |
| 136 | 244251.4 | 2586674T6 | 543 | 974 |
| 136 | 244251.4 | 2586674H1 | 1 | 265 |
| 136 | 244251.4 | 2586674F6 | 1 | 308 |
| 137 | 21555.1 | 2611420H1 | 1 | 251 |
| 137 | 21555.1 | 2611420F6 | 1 | 373 |
| 138 | 234157.3 | 2621425H1 | 939 | 1200 |
| 138 | 234157.3 | 2621425R6 | 939 | 1376 |
| 139 | 903478.1 | 2621425T6 | 1 | 559 |
| 140 | 411296.2 | 2651610T6 | 1667 | 2272 |
| 140 | 411296.2 | 2651610F6 | 1326 | 1846 |
| 140 | 411296.2 | 2651610H1 | 1293 | 1540 |
| 141 | 18044.1 | 2652691F6 | 93 | 574 |
| 141 | 18044.1 | 2652691H1 | 93 | 330 |
| 142 | 407260.2 | 2669708F6 | 768 | 1327 |
| 142 | 407260.2 | 2669708H1 | 768 | 1019 |
| 142 | 407260.2 | 2669708T6 | 906 | 1483 |
| 143 | 978765.2 | 2756450R6 | 1 | 194 |
| 143 | 978765.2 | 2756450T6 | 1 | 156 |
| 144 | 338274.1 | 2830040F6 | 8 | 458 |
| 144 | 338274.1 | 2830040T6 | 1 | 507 |
| 144 | 338274.1 | 2830040H1 | 8 | 265 |
| 145 | 231270.1 | 2905430H1 | 606 | 678 |
| 145 | 231270.1 | 2905430F6 | 606 | 938 |
| 146 | 18092.1 | 2905612H1 | 1 | 190 |
| 146 | 18092.1 | 2905612F6 | 1 | 190 |
| 147 | 404197.8c | 2964015F6 | 16 | 413 |
| 147 | 404197.8c | 2964015H1 | 103 | 413 |
| 148 | 404197.4 | 2964015T6 | 2687 | 3114 |
| 149 | 5460.1 | 3124174T6 | 87 | 566 |
| 149 | 5460.1 | 3124174F6 | 7 | 349 |
| 149 | 5460.1 | 3124174H1 | 7 | 292 |
| 150 | 346511.4 | 3214930F6 | 81 | 650 |
| 151 | 346511.6 | 3214930H2 | 17 | 125 |
| 152 | 331233.2 | 3246843F6 | 1 | 398 |
| 152 | 331233.2 | 3246843H1 | 1 | 231 |
| 153 | 234056.5 | 3561726H1 | 1 | 292 |
| 153 | 234056.5 | 3561726F6 | 1 | 402 |
| 154 | 238118.1 | 220964T6 | 710 | 871 |
| 154 | 238118.1 | 220964H1 | 710 | 912 |
| 154 | 238118.1 | 220964R6 | 710 | 912 |
| 155 | 233596.5 | 1368493R6 | 692 | 1056 |
| 155 | 233596.5 | 1368493T6 | 692 | 1015 |
| 155 | 233596.5 | 1368493T1 | 692 | 1013 |
| 155 | 233596.5 | 1368493H1 | 692 | 924 |
| 156 | 988653.1 | 1705208H1 | 1702 | 1906 |
| 156 | 988653.1 | 1705208F6 | 1702 | 2278 |
| 156 | 988653.1 | 1705208T6 | 1755 | 2382 |
| 157 | 470468.26 | 1806435CB1 | 486 | 1870 |
| 157 | 470468.26 | 1806435F6 | 800 | 1331 |
| 157 | 470468.26 | 1806435H1 | 800 | 1063 |
| 158 | 470468.25 | 1806435T6 | 293 | 806 |
| 159 | 903479.3 | 2013450R6 | 1591 | 1828 |
| 159 | 903479.3 | 2013450H1 | 1591 | 1828 |
| 159 | 903479.3 | 2013450T6 | 1591 | 1786 |
| 160 | 267918.1 | 2275616R6 | 659 | 1034 |
| 160 | 267918.1 | 2275616H1 | 659 | 932 |
| 160 | 267918.1 | 2275616T6 | 659 | 992 |
| 161 | 1094412.1 | 2641522CA2 | 6 | 2512 |
| 162 | 1094107.1 | 2641522T6 | 2095 | 2480 |
| 162 | 1094107.1 | 2641522H1 | 3 | 188 |
| 162 | 1094107.1 | 2641522F6 | 3 | 445 |
| 163 | 218452.4 | 3013646H1 | 3470 | 3750 |
| 163 | 218452.4 | 3013646F6 | 3470 | 3957 |
| 163 | 218452.4 | 3013646T6 | 3795 | 4216 |
| 164 | 12178.2 | 014034H1 | 564 | 839 |
| 165 | 100653.3 | 025818H1 | 431 | 632 |
| 166 | 978478.4 | 041692H1 | 494 | 741 |
| 166 | 978478.4 | 041692R6 | 494 | 831 |
| 167 | 85942.2 | 109658R6 | 44 | 521 |
| 168 | 85942.3 | 109658T6 | 416 | 986 |
| 169 | 977470.16c | 155890T6 | 64 | 477 |
| 169 | 977470.16c | 155890H1 | 1926 | 2123 |
| 169 | 977470.16c | 155890R6 | 1785 | 2123 |
| 170 | 283762.2 | 156350R6 | 1538 | 1813 |
| 170 | 283762.2 | 156350H1 | 1538 | 1713 |
| 171 | 331065.2 | 162793H1 | 1798 | 2217 |
| 172 | 331065.5 | 162793R6 | 1666 | 2059 |
| 173 | 252782.4 | 162793T6 | 2178 | 2789 |
| 174 | 229068.1 | 197779R6 | 359 | 821 |
| 174 | 229068.1 | 197779H1 | 359 | 584 |
| 174 | 229068.1 | 197779T6 | 745 | 1317 |
| 175 | 407450.7 | 275683R6 | 188 | 447 |
| 175 | 407450.7 | 275683H1 | 188 | 432 |
| 175 | 407450.7 | 275683T6 | 280 | 877 |
| 176 | 200163.2 | 348429R6 | 437 | 979 |
| 176 | 200163.2 | 348429R1 | 437 | 917 |
| 176 | 200163.2 | 348429F1 | 915 | 1497 |
| 176 | 200163.2 | 348429T7 | 948 | 1459 |
| 176 | 200163.2 | 348429T6 | 1248 | 1454 |
| 177 | 200163.12c | 348429CB1 | 7 | 1801 |
| 177 | 200163.12c | 348429H1 | 171 | 401 |
| 178 | 1328237.4 | 452790T7 | 2338 | 2685 |
| 178 | 1328237.4 | 452790R6 | 976 | 1423 |
| 178 | 1328237.4 | 452790H1 | 976 | 1211 |
| 178 | 1328237.4 | 452790R7 | 976 | 1406 |
| 179 | 333965.1 | 452790T6 | 1905 | 2426 |
| 180 | 1328236.5 | 515029H1 | 847 | 1071 |
| 180 | 1328236.5 | 515029R6 | 597 | 1070 |
| 180 | 1328236.5 | 515029T6 | 169 | 776 |
| 181 | 995534.3 | 546656H1 | 1514 | 1803 |
| 181 | 995534.3 | 546656R6 | 1514 | 1973 |
| 181 | 995534.3 | 546656T6 | 2012 | 2433 |
| 181 | 995534.3 | 546656CB1 | 70 | 4457 |
| 182 | 1098887.1 | 569710R1 | 191 | 728 |
| 182 | 1098887.1 | 2317772T6 | 922 | 1262 |
| 182 | 1098887.1 | 569710H1 | 190 | 439 |
| 182 | 1098887.1 | 569710R6 | 190 | 729 |
| 182 | 1098887.1 | 569710CB1 | 1 | 1309 |
| 182 | 1098887.1 | 569710CA2 | 191 | 1323 |
| 182 | 1098887.1 | 2317772H1 | 511 | 765 |
| 182 | 1098887.1 | 2317772R6 | 511 | 987 |
| 182 | 1098887.1 | 569710F1 | 691 | 1309 |
| 182 | 1098887.1 | 569710T6 | 813 | 1271 |
| 183 | 268733.1 | 586337H1 | 1 | 239 |
| 183 | 268733.1 | 586337R6 | 1 | 239 |
| 184 | 470023.2 | 610280R6 | 292 | 808 |
| 184 | 470023.2 | 610280H1 | 292 | 563 |
| 185 | 989966.8 | 610280T6 | 1955 | 2086 |
| 186 | 233925.5 | 637639R6 | 833 | 1195 |
| 186 | 233925.5 | 637639H1 | 833 | 1089 |
| 186 | 233925.5 | 637639T6 | 1079 | 1694 |
| 187 | 405145.5 | 855379R1 | 7249 | 7460 |
| 187 | 405145.5 | 855379R6 | 7249 | 7460 |
| 187 | 405145.5 | 855379H1 | 7249 | 7460 |
| 188 | 261982.8 | 1234054CB1 | 698 | 8277 |
| 188 | 261982.8 | 4106629F6 | 992 | 1484 |
| 188 | 261982.8 | 4106629H1 | 994 | 1275 |
| 188 | 261982.8 | 4106629T6 | 2457 | 2747 |
| 188 | 261982.8 | 1234054H1 | 5894 | 6152 |
| 188 | 261982.8 | 1234054F6 | 5894 | 6249 |
| 188 | 261982.8 | 1234054T6 | 6312 | 6832 |
| 189 | 246285.1 | 1275388F6 | 230 | 676 |
| 189 | 246285.1 | 1275388H1 | 230 | 447 |
| 189 | 246285.1 | 1275388T6 | 1699 | 2143 |
| 190 | 228511.1 | 1283340H1 | 3122 | 3366 |
| 190 | 228511.1 | 1283340F6 | 3122 | 3523 |
| 190 | 228511.1 | 1283340T6 | 3719 | 4120 |
| 191 | 40290.1 | 1289539H1 | 754 | 897 |
| 191 | 40290.1 | 1289539F6 | 754 | 981 |
| 191 | 40290.1 | 1289539T6 | 761 | 939 |
| 192 | 399465.3 | 1297406H1 | 1504 | 1749 |
| 192 | 399465.3 | 1297406F1 | 1504 | 1997 |
| 192 | 399465.3 | 1297406F6 | 1504 | 1998 |
| 192 | 399465.3 | 1297406T6 | 3822 | 4041 |
| 193 | 898850.21 | 1307776F6 | 442 | 738 |
| 193 | 898850.21 | 1307776H1 | 501 | 738 |
| 194 | 201356.1 | 1316173T6 | 2198 | 2684 |
| 194 | 201356.1 | 1316173F6 | 429 | 859 |
| 194 | 201356.1 | 1316173H1 | 429 | 685 |
| 195 | 223285.1 | 1362803H1 | 101 | 350 |
| 195 | 223285.1 | 1362803F6 | 101 | 532 |
| 195 | 223285.1 | 1362803F1 | 101 | 550 |
| 196 | 318000.4 | 1382007CA2 | 79 | 1133 |
| 196 | 318000.4 | 1382007F6 | 79 | 516 |
| 196 | 318000.4 | 1382007H1 | 79 | 342 |
| 196 | 318000.4 | 1382007T6 | 574 | 1103 |
| 197 | 977887.1 | 1416679CB1 | 442 | 4897 |
| 197 | 977887.1 | 1416679H1 | 4021 | 4259 |
| 197 | 977887.1 | 1416679F6 | 4021 | 4331 |
| 197 | 977887.1 | 1416679T6 | 4324 | 4837 |
| 198 | 1094199.1 | 1441706F6 | 6769 | 7116 |
| 199 | 1094984.12 | 1441706H1 | 1 | 271 |
| 200 | 244785.3 | 1441706T6 | 1790 | 1857 |
| 201 | 242278.1c | 1445226H1 | 278 | 525 |
| 201 | 242278.1c | 1445226F6 | 278 | 749 |
| 202 | 113621.5 | 1445226T6 | 2851 | 3328 |
| 203 | 401532.2 | 1456482F6 | 5517 | 5677 |
| 203 | 401532.2 | 1456482H1 | 5517 | 5688 |
| 203 | 401532.2 | 1456482T6 | 5518 | 5651 |
| 204 | 006985.1c | 1474660R6 | 437 | 628 |
| 204 | 006985.1c | 1474660H1 | 431 | 628 |
| 204 | 006985.1c | 1474660T6 | 463 | 628 |
| 205 | 351209.14 | 1486467H1 | 1 | 299 |
| 205 | 351209.14 | 1486467F6 | 1 | 509 |
| 206 | 351209.16 | 1486467T6 | 2094 | 2432 |
| 207 | 475819.14 | 1488957T6 | 1989 | 2273 |
| 207 | 475819.14 | 1488957F6 | 1996 | 2319 |
| 207 | 475819.14 | 1488957H1 | 1996 | 2214 |
| 208 | 241384.3 | 1551017T6 | 1850 | 2384 |
| 208 | 241384.3 | 1551017R6 | 31 | 370 |
| 208 | 241384.3 | 1551017H1 | 31 | 233 |
| 209 | 3428.1 | 1560819H1 | 117 | 320 |
| 209 | 3428.1 | 1560819F6 | 117 | 383 |
| 209 | 3428.1 | 1560819T6 | 174 | 382 |
| 210 | 244200.1 | 1599154F6 | 1619 | 2129 |
| 210 | 244200.1 | 1599154H1 | 1619 | 1804 |
| 210 | 244200.1 | 1599154T6 | 2329 | 2722 |
| 211 | 412484.3 | 1600612F6 | 59 | 401 |
| 211 | 412484.3 | 1600612H1 | 59 | 273 |
| 212 | 412484.8 | 1600612T6 | 679 | 1148 |
| 213 | 402187.16 | 1607765F6 | 1033 | 1401 |
| 213 | 402187.16 | 1607765H1 | 1033 | 1239 |
| 213 | 402187.16 | 1607765T6 | 1047 | 1370 |
| 214 | 903091.31 | 1618369CA2 | 145 | 1709 |
| 215 | 903091.33c | 1618369F6 | 2363 | 2905 |
| 215 | 903091.33c | 1618369CB1 | 754 | 2904 |
| 216 | 903091.16 | 1618369H1 | 1 | 219 |
| 217 | 228447.29c | 1639231F6 | 1 | 230 |
| 217 | 228447.29c | 1639231H1 | 1 | 201 |
| 218 | 475473.1 | 1649906H1 | 1155 | 1354 |
| 218 | 475473.1 | 1649906F6 | 1155 | 1543 |
| 219 | 354430.4 | 1661486F6 | 3990 | 4349 |
| 219 | 354430.4 | 1661486H1 | 3990 | 4201 |
| 219 | 354430.4 | 1661486T6 | 6224 | 6432 |
| 220 | 468221.12c | 1662856F6 | 257 | 765 |
| 220 | 468221.12c | 1662856H1 | 257 | 484 |
| 221 | 468221.13 | 1662856T6 | 992 | 1313 |
| 222 | 399187.1c | 1669720F6 | 1 | 398 |
| 222 | 399187.1c | 1669720H1 | 300 | 398 |
| 223 | 903338.12 | 1669720T6 | 157 | 508 |
| 224 | 208529.1 | 1676657T6 | 720 | 1170 |
| 224 | 208529.1 | 1676657F6 | 524 | 900 |
| 224 | 208529.1 | 1676657H1 | 524 | 767 |
| 225 | 1040667.52 | 1684010H1 | 482 | 696 |
| 225 | 1040667.52 | 1684010F6 | 482 | 962 |
| 226 | 147665.1 | 1709754H1 | 492 | 706 |
| 226 | 147665.1 | 1709754F6 | 492 | 787 |
| 226 | 147665.1 | 1709754T6 | 492 | 749 |
| 227 | 218524.4 | 1715437F6 | 1103 | 1570 |
| 227 | 218524.4 | 1715437H1 | 1103 | 1373 |
| 227 | 218524.4 | 1715437T6 | 1352 | 1767 |
| 228 | 346673.1 | 1724856F6 | 2244 | 2611 |
| 228 | 346673.1 | 1724856H1 | 2244 | 2451 |
| 228 | 346673.1 | 1842015H1 | 2744 | 2937 |
| 228 | 346673.1 | 1842015R6 | 2744 | 3100 |
| 228 | 346673.1 | 1724856T6 | 2888 | 3065 |
| 228 | 346673.1 | 1842015T6 | 2959 | 3056 |
| 229 | 58775.1 | 1734355T6 | 1173 | 1562 |
| 229 | 58775.1 | 1734355H1 | 469 | 687 |
| 229 | 58775.1 | 1734355F6 | 469 | 784 |
| 230 | 197086.1 | 1807312F6 | 2059 | 2386 |
| 230 | 197086.1 | 1807312H1 | 2059 | 2338 |
| 230 | 197086.1 | 1807312T6 | 2317 | 2846 |
| 231 | 348143.7 | 1818111CB1 | 1 | 2418 |
| 231 | 348143.7 | 1818111F6 | 153 | 519 |
| 231 | 348143.7 | 1818111H1 | 153 | 414 |
| 232 | 239552.3c | 1831470F6 | 300 | 792 |
| 232 | 239552.3c | 1831470H1 | 676 | 792 |
| 232 | 239552.3c | 1831470T6 | 60 | 245 |
| 233 | 233003.6 | 1834236R6 | 276 | 838 |
| 233 | 233003.6 | 1834236H1 | 276 | 565 |
| 234 | 233003.2 | 1834236T6 | 1 | 552 |
| 235 | 217860.1 | 1843153T6 | 2420 | 2968 |
| 235 | 217860.1 | 1843153H1 | 1269 | 1559 |
| 235 | 217860.1 | 1843153R6 | 1269 | 1531 |
| 236 | 216189.2 | 1859548T6 | 1716 | 2195 |
| 236 | 216189.2 | 1859548F6 | 1261 | 1721 |
| 236 | 216189.2 | 1859548H1 | 1261 | 1521 |
| 237 | 343692.15c | 1869342H1 | 3225 | 3468 |
| 237 | 343692.15c | 1869342F6 | 3018 | 3468 |
| 238 | 382906.16 | 1874037F6 | 54 | 505 |
| 238 | 382906.16 | 1874037H1 | 54 | 289 |
| 239 | 482325.15 | 1877064T6 | 1857 | 2050 |
| 239 | 482325.15 | 1877064H1 | 1864 | 2088 |
| 239 | 482325.15 | 1877064F6 | 1864 | 2090 |
| 240 | 64851.1 | 1903544H1 | 142 | 264 |
| 240 | 64851.1 | 1903544F6 | 142 | 264 |
| 241 | 346016.5 | 1907660F6 | 6 | 527 |
| 241 | 346016.5 | 1907660T6 | 116 | 541 |
| 242 | 346016.6 | 1907660H1 | 26 | 296 |
| 243 | 222222.1 | 1911126H1 | 99 | 258 |
| 243 | 222222.1 | 1911126F6 | 99 | 476 |
| 244 | 413835.5 | 1968816R6 | 3431 | 3682 |
| 244 | 413835.5 | 1968816H1 | 3431 | 3579 |
| 244 | 413835.5 | 1968816T6 | 3431 | 3633 |
| 245 | 978273.4 | 1975523H1 | 1527 | 1794 |
| 245 | 978273.4 | 1975523F6 | 1527 | 1787 |
| 245 | 978273.4 | 1975523T6 | 1536 | 1777 |
| 246 | 474673.1 | 1989435CB1 | 176 | 5407 |
| 246 | 474673.1 | 1989435H1 | 1973 | 2208 |
| 246 | 474673.1 | 1989435F6 | 1973 | 2393 |
| 246 | 474673.1 | 1989435T6 | 3111 | 3690 |
| 247 | 480286.1 | 2044967F6 | 807 | 1062 |
| 248 | 402640.1 | 2044967H1 | 777 | 1036 |
| 249 | 232218.1 | 2070055F6 | 1 | 422 |
| 249 | 232218.1 | 2070055H1 | 1 | 275 |
| 249 | 232218.1 | 2070055T6 | 1292 | 1649 |
| 250 | 981919.1c | 2136337F6 | 1 | 267 |
| 250 | 981919.1c | 2136337T6 | 6 | 256 |
| 250 | 981919.1c | 2136337H1 | 1 | 77 |
| 251 | 229369.1 | 2151736F6 | 155 | 527 |
| 251 | 229369.1 | 2151736H1 | 155 | 399 |
| 251 | 229369.1 | 2151736T6 | 363 | 931 |
| 252 | 253946.17 | 2172334CB1 | 208 | 3544 |
| 252 | 253946.17 | 2172334F6 | 1163 | 1711 |
| 252 | 253946.17 | 2172334H1 | 1163 | 1418 |
| 252 | 253946.17 | 2172334T6 | 1891 | 2457 |
| 253 | 13550.1 | 2190842CB1 | 63 | 584 |
| 253 | 13550.1 | 2190842F6 | 155 | 579 |
| 253 | 13550.1 | 2190842H1 | 155 | 325 |
| 253 | 13550.1 | 2190842T6 | 207 | 537 |
| 254 | 997704.1 | 2191181H1 | 856 | 1086 |
| 254 | 997704.1 | 2191181F6 | 856 | 1089 |
| 254 | 997704.1 | 2191181T6 | 860 | 1044 |
| 255 | 385608.45 | 2219234F6 | 878 | 1327 |
| 256 | 385608.25c | 2219234H1 | 682 | 935 |
| 257 | 385608.3 | 2219234T6 | 3156 | 3676 |
| 258 | 78434.1 | 2308788T6 | 1 | 141 |
| 258 | 78434.1 | 2308788R6 | 1 | 183 |
| 258 | 78434.1 | 2308788H1 | 1 | 193 |
| 259 | 243134.1 | 2355979T6 | 5 | 298 |
| 259 | 243134.1 | 2355979F6 | 9 | 304 |
| 259 | 243134.1 | 2355979H1 | 80 | 304 |
| 260 | 196959.4c | 2358377CB1 | 1222 | 3902 |
| 260 | 196959.4c | 2358377R6 | 3476 | 3902 |
| 260 | 196959.4c | 2358377H1 | 3676 | 3902 |
| 261 | 82168.5 | 2450092F6 | 1 | 441 |
| 261 | 82168.5 | 2450092H1 | 1 | 221 |
| 262 | 4360.1 | 2458924F6 | 172 | 614 |
| 262 | 4360.1 | 2458924H1 | 172 | 402 |
| 262 | 4360.1 | 2458924T6 | 549 | 951 |
| 263 | 412661.2 | 2470485F6 | 481 | 1054 |
| 263 | 412661.2 | 2470485H1 | 481 | 732 |
| 263 | 412661.2 | 172023R6 | 7002 | 7403 |
| 263 | 412661.2 | 172023H1 | 7002 | 7190 |
| 264 | 206310.2 | 2486065H1 | 1747 | 1973 |
| 264 | 206310.2 | 2486065F6 | 1747 | 1836 |
| 265 | 346599.14c | 2515389F6 | 726 | 1170 |
| 265 | 346599.14c | 2515389H1 | 845 | 1170 |
| 266 | 385608.47 | 2520418F6 | 309 | 681 |
| 267 | 385608.2 | 2520418CB1 | 1340 | 4480 |
| 267 | 385608.2 | 2520418H1 | 1330 | 1618 |
| 268 | 335202.1 | 2530672T6 | 438 | 549 |
| 268 | 335202.1 | 2530672F6 | 445 | 719 |
| 268 | 335202.1 | 2530672H1 | 445 | 702 |
| 269 | 978402.3 | 2553094H1 | 263 | 505 |
| 269 | 978402.3 | 2553094F6 | 263 | 605 |
| 270 | 232146.1 | 2578906T6 | 2675 | 2967 |
| 270 | 232146.1 | 2578906H1 | 381 | 624 |
| 270 | 232146.1 | 2578906F6 | 381 | 798 |
| 271 | 25757.1 | 2588149F6 | 110 | 371 |
| 271 | 25757.1 | 2588149H1 | 110 | 340 |
| 271 | 25757.1 | 2588149T6 | 110 | 329 |
| 272 | 206860.2 | 2589404F6 | 1 | 199 |
| 272 | 206860.2 | 2589404H1 | 1 | 199 |
| 272 | 206860.2 | 2589404T6 | 25 | 199 |
| 273 | 239797.3 | 2598317F6 | 656 | 1017 |
| 273 | 239797.3 | 2598317H1 | 656 | 903 |
| 273 | 239797.3 | 2598317T6 | 664 | 984 |
| 274 | 441328.12 | 2601777F6 | 2979 | 3491 |
| 274 | 441328.12 | 2601777H1 | 2979 | 3262 |
| 274 | 441328.12 | 2601777T6 | 2972 | 3454 |
| 275 | 113975.1 | 2628789F6 | 1 | 318 |
| 275 | 113975.1 | 2628789H1 | 1 | 229 |
| 275 | 113975.1 | 2628789T6 | 179 | 507 |
| 276 | 427554.6 | 2646362CB1 | 232 | 936 |
| 276 | 427554.6 | 2646362F6 | 284 | 674 |
| 276 | 427554.6 | 2646362CA2 | 284 | 754 |
| 276 | 427554.6 | 2646362H1 | 284 | 536 |
| 277 | 360130.31c | 2646362T6 | 18 | 523 |
| 278 | 82154.23 | 2651158F6 | 139 | 370 |
| 279 | 82154.24 | 2651158H1 | 2386 | 2546 |
| 280 | 1137293.16 | 2651158T6 | 2341 | 2532 |
| 281 | 994468.3 | 2656117F6 | 704 | 1208 |
| 281 | 994468.3 | 2656117H1 | 705 | 919 |
| 282 | 994532.1 | 2656117T6 | 323 | 701 |
| 283 | 90710.1 | 2662470F6 | 1 | 343 |
| 283 | 90710.1 | 2662470H1 | 1 | 248 |
| 284 | 304359.1c | 2662470T6 | 227 | 587 |
| 285 | 12402.1 | 2703241H1 | 1 | 96 |
| 285 | 12402.1 | 2703241F6 | 1 | 95 |
| 285 | 12402.1 | 2703241T6 | 1 | 55 |
| 286 | 92743.1 | 2703930F6 | 1 | 386 |
| 286 | 92743.1 | 2703930H1 | 1 | 264 |
| 286 | 92743.1 | 2703930T6 | 260 | 586 |
| 287 | 18513.1 | 2704425H1 | 1 | 100 |
| 287 | 18513.1 | 2704425F6 | 1 | 273 |
| 287 | 18513.1 | 2704425T6 | 4 | 425 |
| 288 | 477387.7 | 2747525T6 | 751 | 1255 |
| 288 | 477387.7 | 2747525F6 | 394 | 931 |
| 288 | 477387.7 | 2747525H1 | 394 | 602 |
| 289 | 350440.14c | 2753673H1 | 2861 | 3132 |
| 290 | 350440.15 | 2753673R6 | 1742 | 2190 |
| 291 | 992455.56c | 2762278H1 | 2280 | 2516 |
| 291 | 992455.56c | 2762278F6 | 2097 | 2516 |
| 291 | 992455.56c | 2762278T6 | 430 | 921 |
| 292 | 997395.4 | 2783496T6 | 938 | 1203 |
| 292 | 997395.4 | 2783496H2 | 945 | 1200 |
| 292 | 997395.4 | 2783496F6 | 945 | 1241 |
| 293 | 17821.1 | 2784511H1 | 1 | 218 |
| 293 | 17821.1 | 2784511F6 | 1 | 218 |
| 294 | 201436.4c | 2790307H2 | 857 | 1168 |
| 294 | 201436.4c | 2790307F6 | 688 | 1167 |
| 295 | 201436.3 | 2790307T6 | 1668 | 1994 |
| 296 | 198947.1 | 2817268CB1 | 53 | 1362 |
| 296 | 198947.1 | 2817268F6 | 94 | 568 |
| 296 | 198947.1 | 2817268H1 | 94 | 334 |
| 297 | 1135407.1c | 2817931T6 | 42 | 244 |
| 297 | 1135407.1c | 2817931F6 | 7 | 480 |
| 297 | 1135407.1c | 2817931H1 | 252 | 477 |
| 298 | 200046.1 | 2822608F6 | 58 | 298 |
| 298 | 200046.1 | 2822608H1 | 58 | 345 |
| 299 | 981489.1 | 2822608T6 | 1473 | 1869 |
| 300 | 19362.1 | 2825842F6 | 1 | 307 |
| 300 | 19362.1 | 2825842H1 | 1 | 287 |
| 300 | 19362.1 | 2825842T6 | 2 | 267 |
| 301 | 217116.1 | 2829340H1 | 67 | 333 |
| 301 | 217116.1 | 2829340F6 | 67 | 518 |
| 302 | 330530.1 | 2829340T6 | 8 | 320 |
| 303 | 401213.1 | 2854504F6 | 1050 | 1230 |
| 303 | 401213.1 | 2854504H1 | 1050 | 1230 |
| 303 | 401213.1 | 2854504T6 | 1051 | 1191 |
| 304 | 400280.3 | 2882960H1 | 3152 | 3432 |
| 304 | 400280.3 | 2882960F6 | 3152 | 3529 |
| 305 | 17320.1 | 2886757CB1 | 367 | 1412 |
| 305 | 17320.1 | 2886757H1 | 920 | 1177 |
| 305 | 17320.1 | 2886757F6 | 920 | 1397 |
| 306 | 406182.1c | 2889062F6 | 27 | 429 |
| 306 | 406182.1c | 2889062H1 | 345 | 429 |
| 306 | 406182.1c | 2889062T6 | 1 | 305 |
| 307 | 244603.1 | 2893953F6 | 100 | 569 |
| 307 | 244603.1 | 2893953H1 | 101 | 387 |
| 308 | 107405.1 | 2952116H1 | 176 | 401 |
| 308 | 107405.1 | 2952116F6 | 176 | 452 |
| 309 | 337024.4 | 2962332F6 | 1 | 484 |
| 309 | 337024.4 | 2962332H1 | 2 | 304 |
| 310 | 337024.3 | 2962332T6 | 2194 | 2334 |
| 311 | 481848.1c | 2963871CB1 | 1 | 2455 |
| 311 | 481848.1c | 2963871F6 | 1250 | 1727 |
| 311 | 481848.1c | 2963871H1 | 1428 | 1726 |
| 312 | 208723.1 | 2991483T6 | 1 | 184 |
| 312 | 208723.1 | 2991483F6 | 1 | 210 |
| 312 | 208723.1 | 2991483H1 | 1 | 167 |
| 313 | 64612.1 | 3026161H1 | 1584 | 1857 |
| 313 | 64612.1 | 3026161F6 | 1584 | 1941 |
| 314 | 978402.1 | 3031883F6 | 2697 | 3018 |
| 314 | 978402.1 | 3031883H1 | 2697 | 2985 |
| 315 | 453369.8 | 3032715F6 | 508 | 764 |
| 315 | 453369.8 | 3032715H1 | 511 | 764 |
| 315 | 453369.8 | 3032715T6 | 556 | 771 |
| 316 | 27240.1 | 3037826H1 | 6 | 250 |
| 316 | 27240.1 | 3037826F6 | 6 | 286 |
| 317 | 401290.1c | 3037826T6 | 176 | 424 |
| 318 | 481847.1 | 3075694F6 | 13 | 350 |
| 318 | 481847.1 | 3075694H1 | 13 | 267 |
| 319 | 235636.1 | 3075694T6 | 2259 | 2718 |
| 320 | 994057.7 | 3123879T6 | 684 | 920 |
| 320 | 994057.7 | 3123879H1 | 691 | 956 |
| 320 | 994057.7 | 3123879F6 | 691 | 956 |
| 321 | 405559.1 | 3268843F6 | 1 | 464 |
| 321 | 405559.1 | 3268843H1 | 2 | 240 |
| 322 | 16760.1 | 3268843T6 | 1 | 433 |
| 323 | 198522.1 | 3278190F6 | 798 | 1015 |
| 323 | 198522.1 | 3278190H1 | 800 | 1019 |
| 323 | 198522.1 | 3278190T6 | 799 | 975 |
| 324 | 19366.1 | 3296544F6 | 44 | 650 |
| 324 | 19366.1 | 3296544H1 | 44 | 273 |
| 325 | 002679.7c | 3377371F6 | 1 | 518 |
| 325 | 002679.7c | 3377371H1 | 1 | 258 |
| 326 | 208276.1 | 3396772F6 | 126 | 478 |
| 326 | 208276.1 | 3396772H1 | 126 | 365 |
| 326 | 208276.1 | 3396772T6 | 325 | 557 |
| 327 | 21552.1 | 3493669F6 | 87 | 390 |
| 327 | 21552.1 | 3493669H1 | 89 | 350 |
| 328 | 198087.1 | 3594344T6 | 1147 | 1576 |
| 328 | 198087.1 | 3594344F6 | 628 | 1071 |
| 328 | 198087.1 | 3594344H1 | 629 | 923 |
| 329 | 903269.4 | 3616229T6 | 7273 | 7724 |
| 329 | 903269.4 | 3616229F6 | 2912 | 3371 |
| 329 | 903269.4 | 3616229H1 | 2912 | 3215 |
| 330 | 351166.1 | 3624110H1 | 618 | 730 |
| 330 | 351166.1 | 3624110F6 | 619 | 730 |
| 331 | 344713.3 | 3838748H1 | 50 | 282 |
| 331 | 344713.3 | 3838748F6 | 50 | 466 |
| 331 | 344713.3 | 3838748T6 | 63 | 269 |
| 332 | 167920.1 | 4043301F6 | 97 | 435 |
| 332 | 167920.1 | 4043301H1 | 98 | 246 |
| 333 | 433569.1 | 4051491H1 | 982 | 1248 |
| 333 | 433569.1 | 4051491F6 | 982 | 1601 |
| 333 | 433569.1 | 4051491T6 | 1341 | 1922 |
| 334 | 406365.1 | 4082055F6 | 1 | 276 |
| 334 | 406365.1 | 4082055H1 | 1 | 14 |
| 334 | 406365.1 | 4082055T6 | 207 | 702 |
| 335 | 348121.9 | 4107595F6 | 1952 | 2416 |
| 335 | 348121.9 | 4107595H1 | 1953 | 2205 |
| 336 | 363585.1c | 4190635F6 | 203 | 553 |
| 336 | 363585.1c | 4190635H1 | 314 | 553 |
| 337 | 408116.1 | 4190635T6 | 2 | 145 |
| 338 | 339737.1c | 4252048F6 | 1 | 384 |
| 338 | 339737.1c | 4252048H1 | 146 | 384 |
| 339 | 978146.1 | 4416082H1 | 436 | 673 |
| 339 | 978146.1 | 4416082F6 | 436 | 735 |
| 339 | 978146.1 | 4416082T6 | 512 | 735 |
| 340 | 186012.1 | 4641887F6 | 410 | 697 |
| 340 | 186012.1 | 4641887H1 | 411 | 639 |
| 341 | 405041.1 | 4754771F6 | 411 | 855 |
| 341 | 405041.1 | 4754771H1 | 411 | 679 |
| 342 | 474266.2 | 4754771T6 | 1928 | 2239 |
| 343 | 401530.2 | 4765137H1 | 384 | 643 |
| 343 | 401530.2 | 4765137F6 | 385 | 685 |
| 344 | 234729.11 | 4765137T6 | 433 | 607 |
| 345 | 195199.1 | 4936477F6 | 1178 | 1498 |
| 345 | 195199.1 | 4936477H1 | 1179 | 1394 |
| 345 | 195199.1 | 4936477T6 | 1822 | 2073 |
| 346 | 368731.1 | 5098311T6 | 71 | 515 |
| 346 | 368731.1 | 5098311F6 | 1 | 499 |
| 346 | 368731.1 | 5098311H1 | 1 | 262 |
| 347 | 372313.6 | 5260618F6 | 172 | 611 |
| 347 | 372313.6 | 5260618H1 | 172 | 409 |
| 347 | 372313.6 | 5260618T6 | 387 | 875 |
| 348 | 256871.2 | 5675077H1 | 169 | 286 |
| 348 | 256871.2 | 5675077F6 | 169 | 549 |
| 349 | 256871.16c | 5675077T6 | 219 | 640 |
| 350 | 22485.15 | 342285H1 | 305 | 537 |
| 351 | 22485.1 | 342285R6 | 357 | 859 |
| 351 | 22485.1 | 342285CB1 | 6 | 1038 |
| 352 | 26410.1 | 443631H1 | 44 | 285 |
| 352 | 26410.1 | 443631R6 | 44 | 453 |
| 352 | 26410.1 | 443631T6 | 186 | 521 |
| 353 | 215720.1 | 630607R6 | 1 | 389 |
| 353 | 215720.1 | 630607H1 | 1 | 148 |
| 354 | 472165.22 | 827257R6 | 1 | 338 |
| 354 | 472165.22 | 827257T1 | 2 | 338 |
| 354 | 472165.22 | 827257T6 | 2 | 284 |
| 354 | 472165.22 | 827257R1 | 2 | 338 |
| 354 | 472165.22 | 827257H1 | 1 | 284 |
| 355 | 206580.1 | 919907H1 | 136 | 438 |
| 355 | 206580.1 | 919907R6 | 136 | 615 |
| 355 | 206580.1 | 919907T6 | 204 | 629 |
| 356 | 344775.3 | 1292238F6 | 1929 | 2096 |
| 356 | 344775.3 | 1292238H1 | 1929 | 2095 |
| 356 | 344775.3 | 1292238T6 | 2155 | 2495 |
| 357 | 334668.1 | 1417114F6 | 797 | 956 |
| 357 | 334668.1 | 1417114H1 | 797 | 956 |
| 357 | 334668.1 | 1417114T6 | 797 | 916 |
| 358 | 416874.3 | 1445547H1 | 1416 | 1678 |
| 358 | 416874.3 | 1445547F6 | 1416 | 1949 |
| 358 | 416874.3 | 1445547T6 | 1677 | 2292 |
| 359 | 427964.2 | 1507768T6 | 1138 | 1508 |
| 359 | 427964.2 | 1507768F6 | 1145 | 1548 |
| 359 | 427964.2 | 1507768H1 | 1145 | 1345 |
| 360 | 234537.3c | 1718651H1 | 421 | 608 |
| 360 | 234537.3c | 1718651F6 | 150 | 608 |
| 360 | 234537.3c | 1718651T6 | 31 | 571 |
| 360 | 234537.3c | 2169651T6 | 44 | 556 |
| 360 | 234537.3c | 2169651F6 | 2789 | 3156 |
| 360 | 234537.3c | 2169651H1 | 2915 | 3156 |
| 361 | 31760.1 | 1740072R6 | 1 | 339 |
| 361 | 31760.1 | 1740072H1 | 1 | 242 |
| 361 | 31760.1 | 1740072T6 | 1 | 302 |
| 362 | 336953.7 | 1857708H1 | 5175 | 5437 |
| 362 | 336953.7 | 1857708F6 | 5175 | 5635 |
| 362 | 336953.7 | 1857708T6 | 5518 | 5964 |
| 363 | 444771.2 | 2061119H1 | 321 | 590 |
| 363 | 444771.2 | 2061119R6 | 321 | 619 |
| 363 | 444771.2 | 2061119T6 | 321 | 585 |
| 364 | 449173.16 | 2062092R6 | 2140 | 2505 |
| 364 | 449173.16 | 2062092H1 | 2140 | 2359 |
| 365 | 1523.1 | 2176222F6 | 1 | 481 |
| 365 | 1523.1 | 2176222H1 | 1 | 246 |
| 366 | 239097.1 | 2176222T6 | 15 | 403 |
| 367 | 252747.27 | 2829958H1 | 865 | 1078 |
| 367 | 252747.27 | 2829958F6 | 865 | 1242 |
| 368 | 482490.11 | 2888737F6 | 1 | 255 |
| 368 | 482490.11 | 2888737H1 | 5 | 255 |
| 368 | 482490.11 | 2888737T6 | 39 | 262 |
| 369 | 1000084.27 | 3679667F6 | 1805 | 2138 |
| 369 | 1000084.27 | 3679667H1 | 1986 | 2138 |
| 369 | 1000084.27 | 3679667T6 | 1646 | 2012 |
| 370 | 205607.5 | 009140H1 | 1915 | 2180 |
| 370 | 205607.5 | 009140CB1 | 1 | 3018 |
| 371 | 1089426.1 | 041762H1 | 1 | 82 |
| 372 | 335186.2 | 056197H1 | 351 | 548 |
| 373 | 357276.8 | 391601R6 | 1 | 449 |
| 373 | 357276.8 | 391601T6 | 122 | 594 |
| 374 | 474724.5 | 392166T6 | 3735 | 4307 |
| 374 | 474724.5 | 392166R6 | 3753 | 4182 |
| 375 | 230889.3 | 413767H1 | 790 | 1010 |
| 375 | 230889.3 | 413767R6 | 790 | 1227 |
| 375 | 230889.3 | 413767T6 | 1467 | 2064 |
| 376 | 903909.1 | 660142T6 | 1168 | 1598 |
| 376 | 903909.1 | 660142H1 | 778 | 1006 |
| 376 | 903909.1 | 660142R6 | 784 | 999 |
| 377 | 229298.1 | 1320006T6 | 2658 | 3178 |
| 377 | 229298.1 | 1320006F6 | 2510 | 2966 |
| 378 | 229298.2 | 1320006H1 | 1690 | 1930 |
| 379 | 110678.1 | 1381722H1 | 823 | 920 |
| 379 | 110678.1 | 1381722F6 | 823 | 920 |
| 379 | 110678.1 | 1381722T6 | 825 | 920 |
| 380 | 239093.1 | 1506104T6 | 1 | 466 |
| 380 | 239093.1 | 1506104F6 | 8 | 442 |
| 380 | 239093.1 | 1506104H1 | 8 | 204 |
| 381 | 237963.11c | 1561444F6 | 458 | 805 |
| 381 | 237963.11c | 1561444H1 | 603 | 805 |
| 382 | 237963.8 | 1561444T6 | 1409 | 1797 |
| 383 | 400135.1 | 1562305H1 | 1 | 216 |
| 383 | 400135.1 | 1562305F6 | 1 | 538 |
| 384 | 344398.2 | 1575439T6 | 1733 | 2313 |
| 384 | 344398.2 | 1575439F6 | 2082 | 2454 |
| 384 | 344398.2 | 1575439H1 | 2223 | 2454 |
| 385 | 1086647.1 | 1676601T6 | 632 | 760 |
| 385 | 1086647.1 | 1676601F6 | 633 | 760 |
| 385 | 1086647.1 | 1676601H1 | 633 | 760 |
| 386 | 1682.1 | 1676627T6 | 258 | 635 |
| 386 | 1682.1 | 1676627F6 | 265 | 700 |
| 386 | 1682.1 | 1676627H1 | 265 | 481 |
| 387 | 010190.1c | 1705809F6 | 62 | 330 |
| 387 | 010190.1c | 1705809H1 | 113 | 330 |
| 387 | 010190.1c | 1705809T6 | 62 | 312 |
| 388 | 205311.1 | 1717242F6 | 1 | 355 |
| 388 | 205311.1 | 1717242H1 | 1 | 241 |
| 388 | 205311.1 | 1717242T6 | 40 | 355 |
| 389 | 22429.7 | 1726945F6 | 663 | 1163 |
| 389 | 22429.7 | 1726945H1 | 663 | 886 |
| 389 | 22429.7 | 1726945T6 | 931 | 1340 |
| 390 | 59379.1 | 1728196T6 | 1 | 190 |
| 390 | 59379.1 | 1728196F6 | 8 | 190 |
| 390 | 59379.1 | 1728196H1 | 8 | 198 |
| 391 | 236253.1 | 1812687H1 | 1 | 232 |
| 391 | 236253.1 | 1812687F6 | 1 | 422 |
| 391 | 236253.1 | 1812687T6 | 175 | 575 |
| 392 | 331033.1 | 1970111CB1 | 34 | 2891 |
| 392 | 331033.1 | 1970111F6 | 1092 | 1608 |
| 392 | 331033.1 | 1970111H1 | 1092 | 1378 |
| 392 | 331033.1 | 1970111T6 | 2392 | 2838 |
| 393 | 345533.8c | 2052607F6 | 349 | 451 |
| 393 | 345533.8c | 2052607H1 | 348 | 451 |
| 393 | 345533.8c | 2052607T6 | 390 | 451 |
| 394 | 238342.1 | 2173282T6 | 2531 | 2999 |
| 394 | 238342.1 | 2173282H1 | 2557 | 2790 |
| 394 | 238342.1 | 2173282F6 | 2557 | 2957 |
| 395 | 28936.1 | 2378570F6 | 80 | 580 |
| 395 | 28936.1 | 2378570H1 | 80 | 305 |
| 396 | 347865.5 | 2470377F6 | 1 | 307 |
| 396 | 347865.5 | 2470377H1 | 7 | 105 |
| 397 | 347865.4 | 2470377T6 | 347 | 450 |
| 398 | 40057.2 | 2555937F6 | 487 | 1001 |
| 398 | 40057.2 | 2555937T6 | 502 | 719 |
| 398 | 40057.2 | 2555937H1 | 764 | 1001 |
| 399 | 997301.6 | 2665985H1 | 1 | 217 |
| 399 | 997301.6 | 2665985F6 | 1 | 489 |
| 399 | 997301.6 | 2665985T6 | 275 | 803 |
| 400 | 12235.1 | 2728985F6 | 1 | 428 |
| 400 | 12235.1 | 2728985H1 | 1 | 193 |
| 400 | 12235.1 | 2728985T6 | 1 | 403 |
| 401 | 245496.7 | 2750145H1 | 253 | 517 |
| 401 | 245496.7 | 2750145R6 | 253 | 814 |
| 401 | 245496.7 | 2750145F6 | 365 | 862 |
| 402 | 983262.3c | 2778621F6 | 203 | 574 |
| 402 | 983262.3c | 2778621H1 | 203 | 456 |
| 402 | 983262.3c | 2778621T6 | 335 | 886 |
| 403 | 998084.1 | 2821452H1 | 34 | 347 |
| 403 | 998084.1 | 2821452F6 | 34 | 541 |
| 403 | 998084.1 | 2821452T6 | 171 | 741 |
| 404 | 238071.2 | 2823835T6 | 3140 | 3578 |
| 404 | 238071.2 | 2823835H1 | 2541 | 2820 |
| 404 | 238071.2 | 2823835F6 | 2542 | 3036 |
| 405 | 103930.1 | 2903975H1 | 35 | 272 |
| 405 | 103930.1 | 2903975F6 | 36 | 585 |
| 405 | 103930.1 | 2903975T6 | 434 | 670 |
| 406 | 254068.1 | 2914716F6 | 1 | 438 |
| 406 | 254068.1 | 2914716H1 | 2 | 192 |
| 406 | 254068.1 | 2914716T6 | 25 | 437 |
| 407 | 221812.1 | 2952169F6 | 396 | 768 |
| 407 | 221812.1 | 2952169H1 | 396 | 643 |
| 407 | 221812.1 | 2952169T6 | 388 | 986 |
| 408 | 240129.1 | 2956319F6 | 257 | 421 |
| 408 | 240129.1 | 2956319H1 | 257 | 421 |
| 409 | 230297.1c | 2958047F6 | 1 | 496 |
| 409 | 230297.1c | 2958047T6 | 2 | 453 |
| 409 | 230297.1c | 2958047H1 | 1 | 267 |
| 410 | 347796.7 | 3108506H1 | 395 | 686 |
| 410 | 347796.7 | 3108506F6 | 436 | 687 |
| 411 | 411474.17 | 3143449R6 | 4838 | 5336 |
| 411 | 411474.17 | 3143449H1 | 5028 | 5335 |
| 412 | 027434.1c | 3168342H1 | 1 | 145 |
| 412 | 027434.1c | 3168342F6 | 3 | 145 |
| 413 | 979488.1c | 3168342T6 | 392 | 565 |
| 414 | 213988.1 | 3624062F6 | 9 | 242 |
| 414 | 213988.1 | 3624062H1 | 11 | 218 |
| 414 | 213988.1 | 3624062T6 | 9 | 198 |
| 415 | 263336.62 | 279898T6 | 239 | 581 |
| 415 | 263336.62 | 279898R7 | 261 | 614 |
| 415 | 263336.62 | 279898H1 | 261 | 568 |
| 415 | 263336.62 | 279898R6 | 261 | 608 |
| 416 | 153860.6 | 645136T6 | 222 | 609 |
| 416 | 153860.6 | 645136R6 | 1 | 419 |
| 416 | 153860.6 | 645136H1 | 1 | 247 |
| 417 | 154178.1 | 666098T6 | 1 | 398 |
| 417 | 154178.1 | 666098R6 | 1 | 295 |
| 417 | 154178.1 | 666098H1 | 1 | 201 |
| 418 | 337888.3 | 668460H1 | 1357 | 1560 |
| 418 | 337888.3 | 668460R6 | 1357 | 1538 |
| 418 | 337888.3 | 668460T6 | 1655 | 1872 |
| 419 | 240009.2c | 797955R6 | 1 | 398 |
| 419 | 240009.2c | 797955T6 | 1 | 388 |
| 419 | 240009.2c | 797955H1 | 1 | 123 |
| 420 | 160011.1 | 915572H1 | 1 | 225 |
| 420 | 160011.1 | 915572R6 | 1 | 393 |
| 421 | 332919.4 | 924319R6 | 781 | 1262 |
| 421 | 332919.4 | 924319H1 | 781 | 1142 |
| 422 | 6233.1 | 1357993T6 | 5 | 217 |
| 422 | 6233.1 | 1357993F6 | 8 | 258 |
| 422 | 6233.1 | 1357993H1 | 8 | 214 |
| 423 | 245532.17c | 1738288F6 | 475 | 691 |
| 423 | 245532.17c | 1738288H1 | 471 | 691 |
| 423 | 245532.17c | 1738288T6 | 139 | 579 |
| 424 | 205486.1 | 1743453R6 | 1 | 397 |
| 424 | 205486.1 | 1743453H1 | 1 | 196 |
| 424 | 205486.1 | 1743453T6 | 324 | 608 |
| 425 | 987927.13 | 1798209F6 | 677 | 1233 |
| 425 | 987927.13 | 1798209H1 | 677 | 930 |
| 425 | 987927.13 | 1798209T6 | 2136 | 2698 |
| 426 | 25423.3 | 1816768H1 | 19 | 194 |
| 426 | 25423.3 | 1816768F6 | 19 | 457 |
| 426 | 25423.3 | 1816768T6 | 229 | 726 |
| 427 | 1091415.2 | 2056584R6 | 1217 | 1675 |
| 427 | 1091415.2 | 2056584H1 | 1217 | 1455 |
| 428 | 1091415.16c | 2056584T6 | 178 | 738 |
| 429 | 230058.2 | 2500717H1 | 1707 | 1952 |
| 429 | 230058.2 | 2500717F6 | 1707 | 2165 |
| 429 | 230058.2 | 2500717T6 | 1939 | 2519 |
| 430 | 444648.9 | 2757583H1 | 328 | 585 |
| 430 | 444648.9 | 2757583R6 | 328 | 698 |
| 430 | 444648.9 | 2757583F6 | 328 | 698 |
| 430 | 444648.9 | 2757583CB1 | 268 | 709 |
| 431 | 464689.22 | 2845102F6 | 1659 | 2267 |
| 431 | 464689.22 | 2845102H1 | 1659 | 1900 |
| 432 | 464689.15 | 2845102T6 | 1773 | 2251 |
| 433 | 334809.3c | 2935554H1 | 1058 | 1331 |
| 433 | 334809.3c | 2935554F6 | 831 | 1331 |
| 433 | 334809.3c | 2935554T6 | 38 | 598 |
| 434 | 351241.1 | 2955163H1 | 312 | 587 |
| 434 | 351241.1 | 2955163F6 | 326 | 757 |
| 434 | 351241.1 | 2955163T6 | 346 | 716 |
| 435 | 482336.11 | 5164266H1 | 1 | 205 |
| 435 | 482336.11 | 5164266F6 | 1 | 71 |
| 436 | 482336.31 | 5164266T6 | 1237 | 1458 |
| 437 | 197538.2 | 440472R6 | 585 | 945 |
| 437 | 197538.2 | 440472H1 | 585 | 709 |
| 438 | 197538.8 | 440472T6 | 642 | 1050 |
| 439 | 232048.14 | 548808H1 | 94 | 327 |
| 439 | 232048.14 | 548808R6 | 94 | 632 |
| 439 | 232048.14 | 548808T6 | 216 | 595 |
| 440 | 1092381.1 | 1285892H1 | 5090 | 5310 |
| 441 | 903804.1 | 1287264F6 | 318 | 575 |
| 441 | 903804.1 | 1287264H1 | 336 | 575 |
| 441 | 903804.1 | 1287264F1 | 394 | 575 |
| 442 | 210945.6 | 1288118H1 | 299 | 550 |
| 442 | 210945.6 | 1288118F6 | 299 | 688 |
| 443 | 210945.3c | 1288118CB1 | 1 | 1120 |
| 443 | 210945.3c | 1288118T6 | 539 | 856 |
| 444 | 403448.2 | 1292773T6 | 2224 | 2602 |
| 444 | 403448.2 | 1292773F6 | 2256 | 2647 |
| 444 | 403448.2 | 1292773H1 | 2269 | 2509 |
| 445 | 238263.2 | 1301770F6 | 669 | 1096 |
| 445 | 238263.2 | 1301770H1 | 669 | 913 |
| 445 | 238263.2 | 1301770T6 | 1245 | 1774 |
| 446 | 146382.22 | 1313615F6 | 1107 | 1702 |
| 446 | 146382.22 | 1313615H1 | 1107 | 1339 |
| 447 | 146382.25 | 1313615T6 | 165 | 467 |
| 448 | 345705.7 | 1357056H1 | 58 | 304 |
| 448 | 345705.7 | 1357056F1 | 58 | 490 |
| 448 | 345705.7 | 1357056T6 | 2026 | 2525 |
| 448 | 345705.7 | 1357056F6 | 58 | 509 |
| 448 | 345705.7 | 1357056CA2 | 58 | 2578 |
| 449 | 198212.1 | 1377880F6 | 262 | 706 |
| 449 | 198212.1 | 1377880H1 | 262 | 514 |
| 450 | 198212.6 | 1377880T6 | 1232 | 1600 |
| 451 | 900031.8 | 1402833F6 | 1 | 574 |
| 451 | 900031.8 | 1402833H1 | 1 | 233 |
| 452 | 900031.4 | 1402833T6 | 5643 | 6007 |
| 453 | 475365.4 | 1430483F6 | 443 | 829 |
| 453 | 475365.4 | 1430483H1 | 443 | 695 |
| 453 | 475365.4 | 1430483T6 | 1273 | 1699 |
| 454 | 222810.1 | 1438102T6 | 886 | 1263 |
| 454 | 222810.1 | 1438102F6 | 1 | 459 |
| 454 | 222810.1 | 1438102H1 | 1 | 263 |
| 455 | 1084493.6 | 1503350H1 | 1299 | 1571 |
| 455 | 1084493.6 | 1503350F6 | 1308 | 1629 |
| 455 | 1084493.6 | 1503350T6 | 2679 | 3224 |
| 456 | 979005.2 | 1512017H1 | 2127 | 2297 |
| 456 | 979005.2 | 1512017T6 | 3743 | 4252 |
| 456 | 979005.2 | 1512017F6 | 2127 | 2625 |
| 457 | 346686.23 | 1596060T6 | 2552 | 3060 |
| 457 | 346686.23 | 1596060H1 | 2040 | 2231 |
| 457 | 346686.23 | 1596060F6 | 2040 | 2477 |
| 458 | 234568.25 | 1624988H1 | 33 | 108 |
| 458 | 234568.25 | 1624988F6 | 33 | 458 |
| 459 | 234568.17 | 1624988T6 | 2421 | 3023 |
| 460 | 978276.1c | 1639657H1 | 387 | 546 |
| 460 | 978276.1c | 1639657F6 | 278 | 546 |
| 461 | 26968.1 | 1663359F6 | 1 | 468 |
| 461 | 26968.1 | 1663359H1 | 1 | 225 |
| 462 | 243103.24 | 1664686T6 | 907 | 1496 |
| 462 | 243103.24 | 1664686F6 | 112 | 673 |
| 462 | 243103.24 | 1664686H1 | 112 | 342 |
| 463 | 469883.1 | 1670240H1 | 3910 | 4101 |
| 463 | 469883.1 | 1670240F6 | 3910 | 4382 |
| 463 | 469883.1 | 1670240T6 | 3961 | 4578 |
| 464 | 903565.14c | 1672574T6 | 43 | 352 |
| 464 | 903565.14c | 1672574F6 | 2490 | 3031 |
| 464 | 903565.14c | 1672574H1 | 2830 | 3031 |
| 465 | 227932.2 | 1674711H1 | 4373 | 4511 |
| 466 | 903691.6 | 1706278H1 | 2489 | 2717 |
| 466 | 903691.6 | 1706278F6 | 2489 | 2870 |
| 467 | 012995.19c | 1706278T6 | 57 | 561 |
| 468 | 407938.5 | 1727746T6 | 7648 | 8083 |
| 468 | 407938.5 | 1727746H1 | 5870 | 6083 |
| 468 | 407938.5 | 1727746F6 | 5870 | 6174 |
| 469 | 233778.1 | 1748894F6 | 271 | 723 |
| 469 | 233778.1 | 1748894H1 | 271 | 527 |
| 469 | 233778.1 | 1748894T6 | 544 | 792 |
| 470 | 474630.19 | 1807178F6 | 2975 | 3418 |
| 470 | 474630.19 | 1807178H1 | 2975 | 3240 |
| 471 | 217319.7 | 1808080H1 | 1303 | 1574 |
| 471 | 217319.7 | 1808080F6 | 1303 | 1603 |
| 471 | 217319.7 | 1808080T6 | 1719 | 2059 |
| 472 | 1092445.1 | 1808250F6 | 2139 | 2619 |
| 472 | 1092445.1 | 1808250H1 | 2363 | 2628 |
| 473 | 199433.3 | 1852003F6 | 51 | 377 |
| 473 | 199433.3 | 1852003H1 | 51 | 320 |
| 473 | 199433.3 | 1852003T6 | 625 | 761 |
| 474 | 228860.6 | 1857460F6 | 92 | 510 |
| 474 | 228860.6 | 1857460H1 | 92 | 342 |
| 475 | 228860.4c | 1857460T6 | 456 | 1028 |
| 476 | 410688.3 | 1891428F6 | 1 | 219 |
| 476 | 410688.3 | 1891428H1 | 1 | 279 |
| 477 | 347005.5 | 1929865F6 | 6818 | 7115 |
| 477 | 347005.5 | 1929865H1 | 6819 | 7081 |
| 477 | 347005.5 | 1929865T6 | 6818 | 7074 |
| 478 | 334621.13 | 1965915R6 | 329 | 735 |
| 478 | 334621.13 | 1965915H1 | 329 | 611 |
| 478 | 334621.13 | 1965915T6 | 413 | 975 |
| 479 | 28180.1 | 1970545F6 | 411 | 817 |
| 479 | 28180.1 | 1970545H1 | 411 | 653 |
| 479 | 28180.1 | 1970545T6 | 425 | 814 |
| 480 | 252570.6c | 1972687T6 | 501 | 1044 |
| 480 | 252570.6c | 1972687F6 | 3 | 437 |
| 480 | 252570.6c | 1972687H1 | 3 | 264 |
| 481 | 466402.116 | 2025345R6 | 1118 | 1458 |
| 481 | 466402.116 | 2025345T6 | 1118 | 1419 |
| 481 | 466402.116 | 2025345H1 | 1118 | 1378 |
| 482 | 1096160.26c | 2152021F6 | 961 | 1348 |
| 482 | 1096160.26c | 2152021H1 | 961 | 1209 |
| 482 | 1096160.26c | 2152021T6 | 3034 | 3327 |
| 482 | 1096160.26c | 2152021CB1 | 1 | 3006 |
| 483 | 899612.2 | 2188034F6 | 404 | 865 |
| 483 | 899612.2 | 2188034H1 | 404 | 715 |
| 484 | 254107.1 | 2198878F6 | 1811 | 2268 |
| 484 | 254107.1 | 2198878H1 | 1811 | 2055 |
| 484 | 254107.1 | 2198878T6 | 2597 | 3123 |
| 485 | 208328.1 | 2200534F6 | 2103 | 2527 |
| 485 | 208328.1 | 2200534H1 | 2103 | 2317 |
| 486 | 130157.2 | 2295946T6 | 2207 | 2554 |
| 486 | 130157.2 | 2295946R6 | 1310 | 1772 |
| 486 | 130157.2 | 2295946H1 | 1310 | 1459 |
| 487 | 985824.2c | 2330285T6 | 1252 | 1591 |
| 487 | 985824.2c | 2330285H1 | 1252 | 1507 |
| 487 | 985824.2c | 2330285R6 | 1252 | 1634 |
| 488 | 233089.1 | 2350723T6 | 1921 | 2022 |
| 488 | 233089.1 | 2350723F6 | 1928 | 2064 |
| 489 | 336256.1 | 2443920H1 | 1681 | 1863 |
| 489 | 336256.1 | 2443920F6 | 1681 | 2077 |
| 489 | 336256.1 | 2443920T6 | 2982 | 3515 |
| 490 | 979616.2 | 2454756F6 | 458 | 830 |
| 490 | 979616.2 | 2454756H1 | 458 | 692 |
| 490 | 979616.2 | 2454756T6 | 881 | 1322 |
| 491 | 351432.3 | 2457793H1 | 1332 | 1537 |
| 491 | 351432.3 | 2457793F6 | 1332 | 1814 |
| 491 | 351432.3 | 2457793T6 | 4870 | 5364 |
| 492 | 83495.1 | 2499861F6 | 1 | 378 |
| 492 | 83495.1 | 2499861H1 | 1 | 255 |
| 493 | 332595.1 | 2500879F7 | 1 | 368 |
| 493 | 332595.1 | 2500879H1 | 1 | 247 |
| 493 | 332595.1 | 2500879F6 | 1 | 543 |
| 494 | 481251.3 | 2506154T6 | 967 | 1069 |
| 494 | 481251.3 | 2506154F6 | 1 | 313 |
| 494 | 481251.3 | 2506154H1 | 47 | 249 |
| 495 | 25612.1 | 2582525F6 | 221 | 371 |
| 495 | 25612.1 | 2582525H1 | 221 | 516 |
| 496 | 96700.1 | 2788603F6 | 1 | 519 |
| 496 | 96700.1 | 2788603H1 | 1 | 231 |
| 497 | 230048.1 | 2788603T6 | 142 | 663 |
| 498 | 073621.3c | 2878629F6 | 1690 | 1992 |
| 499 | 344582.19 | 2878629H1 | 1 | 216 |
| 500 | 997526.4 | 2891590F6 | 1 | 416 |
| 500 | 997526.4 | 2891590H1 | 1 | 262 |
| 501 | 997526.1 | 2891590T6 | 2443 | 2854 |

What is claimed is:

1. A combination comprising a plurality of cDNAs, wherein the cDNAs are selected from SEQ ID NOs:1-501 and the complements of SEQ ID NOs:1-501.

2. The combination of claim 1, wherein each of the cDNAs is downregulated at least two-fold and is selected from SEQ ID NOs:1-56, 87-153, 164-349, 370-414, and 437-501 and the complements of SEQ ID NOs:1-56, 87-153, 164-349, 370-414, and 437-501.

3. The combination of claim 1, wherein each of the cDNAs is upregulated at least two-fold and is selected from SEQ ID NOs:57-86, 154-163, 350-369, and 415-436 and the complements of 57-86, 154-163, 350-369, and 415-436.

4. The combination of claim 1, wherein the cDNAs are immobilized on a substrate.

5. An isolated cDNA selected from SEQ ID NOs: 14, 26, 40, 52, 55, 60, 65, 68, 73, 79, 82, 85, 92, 110, 112, 114, 115, 117, 122, 125, 126, 130, 136, 137, 139, 141, 143, 144, 145, 146, 147, 160, 164, 166, 167, 168, 190, 191, 194, 195, 199, 201, 204, 211, 212, 222, 224, 226, 229, 233, 234, 240, 243, 245, 248, 250, 253, 254, 259, 264, 268, 269, 270, 272, 276, 278, 279, 281, 282, 284, 285, 286, 293, 296, 297, 299, 300, 301, 362, 306, 308, 312, 313, 314, 317, 319, 321, 321, 322, 322, 323, 324, 325, 326, 330, 331, 332, 334, 336, 337, 338, 339, 340, 342, 346, 353, 355, 357, 361, 365, 366, 371, 372, 376, 380, 383, 385, 386, 387, 390, 399, 400, 402, 405, 406, 408, 409, 410, 412, 413, 417, 418, 419, 420, 422, 422, 424, 426, 438, 444, 445, 453, 456, 460, 461, 471, 479, 480, 487, 490, 492, 495, 496, and 497 and the complements thereof.

6. A method for detecting differential expression of one or more cDNAs in a sample containing nucleic acids, the method comprising:
a) hybridizing the substrate of claim 4 with the nucleic acids, thereby forming one or more hybridization complexes;
b) detecting hybridization complex formation; and
c) comparing the complexes so formed with those of a standard, wherein differences between the standard and sample complex formation indicate differential expression of cDNAs in the sample.

7. The method of claim 6, wherein the sample is from prostate.

8. The method of claim 6, wherein differential expression is diagnostic of prostate cancer.

9. A method for screening a plurality of molecules or compounds to identify a ligand which specifically binds the cDNA, the method comprising:
a) combining the combination of claim 1 with the plurality of molecules or compounds under conditions to allow specific binding; and
b) detecting specific binding between each cDNA and at least one molecule or compound, thereby identifying a ligand that specifically binds each cDNA.

10. The method of claim 9 wherein the plurality of molecules or compounds are selected from DNA molecules, enhancers, mimetics, RNA molecules, peptide nucleic acids, peptides, repressors and transcription factors.

11. A vector containing the cDNA of claim 5.

12. A host cell containing the vector of claim 11.

13. A method for producing a protein, the method comprising:
a) culturing the host cell of claim 12 under conditions for expression of protein; and
b) recovering the protein from the host cell culture.

14. A protein produced by the method of claim 13.

15. A method for using a protein to screen a plurality of molecules or compounds to identify at least one ligand which specifically binds the protein, the method comprising:
a) combining the protein of claim 14 with the plurality of molecules or compounds under conditions to allow specific binding; and
b) detecting specific binding between the protein and a molecule or compound, thereby identifying a ligand which specifically binds the protein.

16. The method of claim 15 wherein the plurality of molecules or compounds is selected from agonists, antagonists, antibodies, DNA molecules, mimetics, peptide nucleic acids, peptides, proteins, or pharmaceutical agents, RNA molecules, and small drug molecules.

17. A composition comprising the protein of claim 14 and a pharmaceutical carrier.

18. A method of using a protein to produce and purify an antibody, the method comprising:
a) immunizing an animal with the protein of claim 14 under conditions to elicit an antibody response;
b) obtaining a sample containing antibodies;
c) combining the sample with the protein under conditions to allow specific binding;
d) recovering the bound protein; and
e) separating the protein from the antibody, thereby obtaining purified antibody that specifically binds the protein.

19. A purified antibody produced by the method of claim 18.

20. A method of using an antibody to detect prostate cancer, the method comprising:
a) contacting a sample with the antibody of claim 19 under conditions to form an antibody:protein complex;
b) detecting antibody:protein complex formation; and
c) comparing complex formation with standards, wherein complex formation indicates the presence of prostate cancer in the sample.
